(19) United States
(12) Patent Application Publication
(10) Pub. No.: US 2024/0156958 A1
(43) Pub. Date: May 16, 2024

(54) SELF-ADJUVANTING MULTI-PROTEIN COMPLEXES FOR MODULAR VACCINE PRODUCTION

(71) Applicant: Design-Zyme LLC, Lawrence, KS (US)

(72) Inventors: Peter Albert Petillo, Lawrence, KS (US); Dwight O'Dell Deay, III, Lawrence, KS (US); Mary Beth Carter, Lawrence, KS (US)

(21) Appl. No.: 18/499,765

(22) Filed: Nov. 1, 2023

Related U.S. Application Data
(60) Provisional application No. 63/382,020, filed on Nov. 2, 2022.

(51) Int. Cl. A61K 39/39; A61K 39/018; A61K 39/02; A61K 39/145; A61K 39/21

(52) U.S. Cl. CPC A61K 39/39; A61K 39/018; A61K 39/0208; A61K 39/145; A61K 39/21; A61K 2039/55555

(57) ABSTRACT

The present invention is broadly concerned with a vaccine composition comprising a central carrier, at least one linear carbohydrate molecule, and at least one immunogen molecule, wherein each of the at least one linear carbohydrate molecule and at least one immunogen molecule are covalently bound to the carrier via respective covalent linkages. Vaccine compositions comprising multivalent carriers and related methods may find various therapeutic and prophylactic applications for inducing an immune response against, treating, or preventing a bacterial, viral, fungal, or protozoan infection, including, but are not limited to, coronaviruses, Lyme Disease, Chlamydia, and the related diseases thereof.

### RELATED APPLICATIONS

The present application claims the priority benefit of U.S. Provisional Patent Application Ser. No. 63/382,020, filed Nov. 2, 2022, entitled SELF-ADJUVANTING MULTI-PROTEIN COMPLEXES FOR MODULAR VACCINE PRODUCTION, incorporated by reference in its entity herein.

### FIELD OF INVENTION

The present invention relates generally to the field of synthetic vaccines, in particular to the formation of stabilized self-adjuvanting multi-protein complexes for modular vaccine production.

### BACKGROUND OF INVENTION

Vaccination constitutes one of the most cost-effective preventative measures against illness and death from infection. Conventional vaccine production techniques using whole pathogens as a vaccine candidate have now transitioned to using subunit components such as recombinant proteins, peptides, or polysaccharides derived from the pathogens. However, subunit components such as proteins, peptides, or polysaccharides alone are poorly immunogenic as they are not easily recognized by immune cells as a foreign body. These immunogens tend to have a low permeability, and low oral and mucosal absorption due to their high molecular weight and hydrophilic character. In addition, both proteins and peptides are susceptible to enzymatic degradation, conferring short half-lives in vivo. Consequently, subunit vaccines need the assistance of adjuvants and/or delivery systems (Bashiri et al., 2020).

Adjuvants are immunomodulatory compounds that are used with immunogens (antigens) in vaccine formulations to increase, improve, or boost an immune response (Li et al., 2021). Adjuvants as a vaccine delivery system can protect antigens, provide sustained release of antigens, target antigens to local lymph nodes, and facilitate immune responses against delivered antigens (Zinkernagel et al. 1997). Moreover, immunomodulatory adjuvants stimulate cellular uptake of antigens from administration sites, activate antigen-presenting cells (APCs) such as B-cells and T-cells, and up-regulate cytokines and chemokines to provide a robust adaptive immune response (Fearon, 1997). Currently, the only FDA-approved adjuvants for use in humans are aluminum salts, AS03, AS04 (Monophosphoryl lipid A (MPL)+aluminum salt), MF59 (oil in water emulsion composed of squalene), AS01_{B} (monophosphoryl lipid A (MPL) and QS-21, combined in a liposomal formulation), and CpG 1018 (cytosine phosphoguanine (CpG), a synthetic form of DNA that mimics bacterial and viral genetic material). These adjuvants are only approved for administration via injection. To date, there are no adjuvants approved for the mucosal delivery of vaccines in the U.S. Mucosal modes of administration include, but are not limited to, oral, intranasal, intravaginal, intrarectal, intraocular, and intravitreal. None of the currently approved adjuvants can be covalently attached to the vaccine unit. Adjuvanted vaccines can cause local reactions, such as redness, swelling, induration, and pain at the injection site, and systemic reactions, such as fever, chills, rashes, and body aches. (Hervé et al., 2019). There is a recognized need in the art for safe adjuvants which can be co-administered as part of a vaccine composition in order to stimulate a response by the immune system to the antigen or antigens that are also part of the vaccine composition. The adjuvant helps the immune system to generate a more robust antibody response to the antigen or antigens than would be seen if the antigen or antigens were injected alone.

Currently almost all vaccines are administered by injection. While injection is effective, the use of needles carries the risks of both infection at the injection site and transmission of infectious diseases, the treatment of which incurs very significant costs. In addition, trained personnel are required to administer vaccines by injection, due to the aforementioned risks. These problems are particularly relevant in low and middle income countries (LMICs). An advantage of mucosal delivery of a vaccine is that it can be delivered other than by way of injection through needles, thereby providing an immunization regime which may be much safer and more suited to mass immunization, and therefore more attractive to mass vaccination in LMICs. This is especially true for the oral or nasal delivery of a vaccine.

More than 90% of all infections use the mucosa as portals of entry. Advantages of mucosal immunization include 1) local production of secretory immunoglobulin A (sIgA) which may block epithelial colonization and penetration of pathogens into the body; 2) immunization of one mucosal site often induces immune response in other mucosal effector tissues (Lawson et al., 2011); and 3) the production of mucosal antibodies (IgA) can prevent systemic infection (Gupta et al., 2015, MacPherson et al., 2008). While complete protection against many infectious agents would, in addition, require the induction of systemic humoral immunity (particularly IgG antibodies) and cytotoxic T lymphocytes (CTLs), generation of sIgA, at the mucosa, especially the nasal mucosa, may also result in reduced disease transmission.

Adjuvants that work for systemic immunization, such as alum, are generally not effective for mucosal immunization. Moreover, traditionally administered vaccines do not promote high/effective levels of mucosal immunity.

Carbohydrates, including linear carbohydrates, have been investigated for their potential as adjuvants in the context of vaccines. Linear carbohydrates that are suitable as adjuvants can be derived from various sources, such as polysaccharides from bacteria or fungi. Carbohydrate-based adjuvants often work by interacting with immune cells called antigen-presenting cells (APCs), such as dendritic cells. They can activate Toll-like receptors (TLRs) or other pattern recognition receptors (PRRs) on these cells, triggering a signaling cascade that leads to the activation of immune responses. While carbohydrate-based adjuvants are still in the experimental stage for many vaccines, some vaccines that use carbohydrates as adjuvants have been developed and approved. For example, the Haemophilus influenzae type b (Hib) vaccine uses a conjugate of the Hib polysaccharide and a carrier protein as an adjuvant.

Hyaluronic acid (HyA) is a natural polysaccharide with a linear structure of repeating disaccharide units composed of D-glucuronic acid and N-acetyl-D-glucosamine. HyA has proven clinical safety in humans, as it has been widely used for medical products (Becker et al., 2009). More recently, attempts have been made to use HyA as a vaccine adjuvant, as HyA can act as both an immunostimulatory agent and vaccine delivery system. JPH05163161 discloses a vaccine composition for intranasal inoculation which consists of an influenza vaccine and hyaluronic acid, or salt thereof, wherein the HyA is not covalently attached to the vaccine. KR 20150140149 discloses either reductive amination to randomly oxidized glucuronic rings or amidation with the carboxylic acid moiety of a glucuronic acid to form HyA-peptide conjugates for transdermal or transmucosal delivery. The conjugate contains 1 to 10 molecules of peptide per hyaluronic acid. U.S. 2021/0393758 and U.S. Pat. No. 9,034,624 disclose derivatizing a GAG through the carboxylic acid moiety of a glucuronic acid with a linker terminated in an aldehyde moiety. This moiety can then be reductively aminated with an amine moiety of a biologically active molecule, typically the N-terminus of a polypeptide or protein chain. U.S. Pat. No. 6,824,793 discloses that the mucosal delivery of esterified auto-crosslinked HyA polymers, in combination with an antigen of interest, acts to enhance the immunogenicity of the co-administered antigen. The HyA derivatives are provided as microspheres that either adsorb or physically incorporate the antigen and provide the best results when co-administered with an adjuvant. Suzuki et al. disclose HyA-coated micelles containing antigens and adjuvants for nasal delivery. In all cases, these attempts to create a HyA adjuvant have drawbacks which are overcome by the present invention.

Isopeptide bonds are amide bonds formed between carboxyl/carboxamide and amino groups, where at least one of the carboxyl or amino groups is outside of the protein main chain (the backbone of the protein). Such bonds are chemically irreversible under biological conditions and are resistant to most proteases.

Proteins that are capable of spontaneous isopeptide bond formation have been advantageously used to develop peptide tag/binding partner pairs which covalently bind to each other, and which provide irreversible interactions (see e.g., WO2011/098772). In this respect, proteins which are capable of spontaneous isopeptide bond formation may be expressed and/or synthesized as separate fragments, to give a peptide tag and a binding partner for the peptide tag, where the two fragments are capable of covalently reconstituting by isopeptide bond formation. The isopeptide bond formed by the peptide tag and binding partner pairs is stable under conditions where non-covalent interactions would rapidly dissociate, e.g. over long periods of time (e.g. weeks), at high temperature (to at least 95° C.), at high force, or with harsh chemical treatment (e.g. pH 2-11, organic solvent, detergents, or denaturants) (see e.g., U.S. Pat. No. 10,526,379).

In brief, a peptide tag/binding partner pair may be derived from any protein or pair of proteins capable of spontaneously forming an isopeptide bond (an isopeptide protein), wherein the proteins are expressed and/or synthesized separately to produce a peptide tag that contains one of the residues involved in the isopeptide bond (e.g. a lysine) and a peptide binding partner that contains the other residue involved in the isopeptide bond (e.g., an asparagine, aspartate, or glutamate residue). It has also been found that it is possible to express the domains comprising the residues involved in isopeptide bond formation separately, i.e., as three separate peptides (domains, modules, or units). In this respect, the peptide tag comprises one of the residues involved in the isopeptide bond (e.g., a lysine), the peptide binding partner comprises the other residue involved in the isopeptide bond (e.g., an asparagine or aspartate), and a third peptide comprises the one or more other residues involved in catalyzing isopeptide bond formation. Mixing all three peptides results in the formation of an isopeptide bond between the two peptides comprising the residues that react to form the isopeptide bond, i.e., the peptide tag and binding partner. Thus, the third peptide mediates the conjugation of the peptide tag and binding partner but does not form of the part resultant structure, i.e., the third peptide is not covalently linked to the peptide tag or binding partner. As such, the third peptide may be viewed as a protein ligase or peptide ligase. This is particularly useful as it minimizes the size of the peptide tag and binding partner that need to be fused to the protein of interest, thereby reducing the possibility of unwanted interactions caused by the addition of the peptide tag or binding partner, e.g., misfolding (see U.S. Pat. Nos. 10,526,379 and 10,889,622).

Various proteins which are capable of spontaneously forming one or more isopeptide bonds (a so-called “isopeptide protein”) have been identified and may be modified to produce a peptide tag/binding partner pair and optionally a peptide ligase, as discussed above. Further proteins that are capable of spontaneously forming one or more isopeptide bonds may be identified by comparing their structures with those of proteins which are known to spontaneously form one or more isopeptide bonds. In particular, other proteins which may spontaneously form an isopeptide bond may be identified by comparing their crystal structures with those from known isopeptide proteins, e.g., the major pilin protein Spy0128, and in particular comparing the lysine followed by asparagine or aspartic acid followed by either a glutamic acid or another aspartic acid residues often involved in the formation of an isopeptide protein. Additionally, other isopeptide proteins may be identified by screening for structural homologues of known isopeptide proteins using the Protein Data Bank using standard database searching tools. The SPASM server may be used to target the 3D structural template of lysine followed by asparagine or aspartic acid followed by either a glutamic acid or another aspartic acid of the isopeptide bond or isopeptide proteins may also be identified by sequence homology alone. Notably, proteins which form isopeptide bonds may be designed de novo as described in WO2011/098772. Rosetta can be used to design isopeptide proteins de novo. (See also Das et al. 2008). Additionally, the RASMOT-3D PRO server can be used to search the protein database for appropriate orientation of residues.

### SUMMARY OF THE INVENTION

The present invention is broadly concerned with a vaccine composition comprising a central carrier, at least one linear carbohydrate molecule, and at least one immunogen molecule, wherein each of the at least one linear carbohydrate molecule and at least immunogen molecule are covalently bound to the central carrier via respective covalent linkages. In some embodiments, each linear carbohydrate molecule and immunogen molecules are covalently bound to the carrier via a respective peptide tag, for use with a binding partner on the carrier, in which the formation of the peptide tag/binding partner pair results in the spontaneous formation of an isopeptide bond between the peptide tag on the carbohydrate molecule or immunogen molecule and the binding partner on the carrier. In another embodiment, the present invention provides a method for producing a self-assembling nanoparticle complex in which the self-assembling nanoparticle is formed through covalent bonding of the binding partner(s) extending from the carrier and the corresponding peptide tags covalently attached to respective linear carbohydrate molecules and immunogen molecules. In some embodiments, a vaccine composition comprising a carrier covalently attached to one or more linear carbohydrate molecules and to a plurality of immunogen molecules via the binding partner/peptide tag pair is formed. In some embodiments, the central carrier is a multivalent carrier that presents a plurality of binding partners providing a plurality of binding sites to attach the linear carbohydrate molecules and/or immunogens. The number of linear carbohydrate molecules and immunogen molecules covalently bound through a peptide tag/binding partner pair can be the same or different on the central carrier. The number of linear carbohydrate molecules each covalently bound through a peptide tag/binding partner pair on a carrier is at least one, at least two, at least three, at least four, at least five, up to the total number of binding sites on the nanoparticle carrier. In another embodiment, a kit comprising linear carbohydrate molecules covalently bound through a peptide tag/binding partner pair to a central carrier have predefined loadings of the linear carbohydrate.

### BRIEF DESCRIPTION OF THE DRAWINGS

FIG. 1 represents a schematic depiction of (A) the peptide tag-linker-carbohydrate molecule and (B) the peptide tag-immunogen molecule.

FIG. 2 is a schematic depiction of a carrier with peptide tagged-linker-carbohydrate molecules and peptide tagged-immunogen molecules, where the covalent binding partner, e.g., the binding site for the peptide tag on the carrier or equivalently the site of peptide attachment, is part of each mi3 monomer and forms an isopeptide bond with the peptide tagged-linker-carbohydrate and peptide tagged-immunogen molecules to create the nanoparticle complex.

FIG. 3 is a pictorial representation of the process of first adding the HyA-SpyTag peptide to a mi3-SpyCatcher nanoparticle, and then adding SpyTag immunogens to create a mosaic-8-RBD-mi3 self-adjuvanting nanoparticle vaccine complex.

FIG. 4 is a representative SDS-PAGE gel showing an HyA(12K)-SpyTag titrated into SpyCatcher-mi3 to form a self-adjuvanting nanoparticle after (A) after 0.5 hours of incubation and (B) after 2 hours of incubation.

FIG. 5 is a fluorescence analysis of the gels in FIG. 3 demonstrating that the degree of HyA attachment to a mi3 nanoparticle can be controlled and varied.

FIG. 6 is a representative SDS-PAGE gel showing (A) a HyA(12K)-SpyTag titrated into SpyCatcher-mi3 to form a self-adjuvanting nanoparticle and (B) HyA(50K)-SpyTag titrated into SpyCatcher-mi3 to form a self-adjuvanting nanoparticle.

FIG. 7 is a DLS analysis of a SpyCatcher modified mi3 nanoparticle (mi3(sc)), a SpyCatcher modified mi3 nanoparticle attached to an unmodified SpyTag peptide (mi3(sc+st)), a SpyTag peptide modified with covalently attached hyaluronic acid tetrasaccharide (HyA2-mi3(sc+st)), and a SpyTag peptide modified with covalently attached hyaluronic acid of a nominal weight of 12 kDa (HyA(12K)-mi3(sc+st)).

FIG. 8 is a zeta potential analysis of a SpyCatcher modified mi3 nanoparticle (mi3(sc)), a SpyCatcher modified mi3 nanoparticle attached to an unmodified SpyTag peptide (mi3(sc+st)), a SpyTag peptide modified with covalently attached hyaluronic acid tetrasaccharide (HyA2-mi3(sc+st)), and a SpyTag peptide modified with covalently attached hyaluronic acid of a nominal weight of 12 kDa (HyA(12K)-mi3(sc+st)).

FIG. 9 is the effect of HyA content on the yield and solubility of the result of HyA-SpyTag addition to the mi3 nanoparticle scaffold.

FIG. 10 is a pictorial representation of HyA-SpyTag added to mi3-SpyCatcher nanoparticle at different loadings of the HyA-SpyTag peptide. The numbers below each image are example percentages of HyA-SpyTag peptide loaded onto the mi3 nanoparticle scaffold.

FIG. 11 is a pictorial representation of varying amounts of HyA-SpyTag peptide on a mosaic-8-OspC-mi3 nanoparticle. The numbers below each image are example percentages of HyA-SpyTag peptide loaded onto the mi3 nanoparticle scaffold, which for (A) is 0% HyA loading, (B) 5% HyA loading, (C) 10% HyA loading, and (D) 20% HyA loading.

### DETAILED DESCRIPTION OF THE PREFERRED EMBODIMENTS

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein. All patents, published patent applications, other publications, and sequences from GenBank and other databases referred to herein, are incorporated by reference in their entirety with respect to the related technology.

Adjuvants are immunomodulatory compounds that are used with immunogens (antigens) in vaccine formulations to increase, improve, or boost an immune response. One skilled in the art would recognize that while traditional vaccines are formulated into mixtures of an immunogen (antigen) plus an adjuvant, vaccines in which the two moieties are contained within a single molecule are designated as self-adjuvanting vaccines. Thus, in the present invention, self-adjuvanting refers to an adjuvant that is covalently attached to the immunogen carrier or the immunogen. By virtue of the covalent attachment, the adjuvant cannot diffuse away from the immunogen carrier or the immunogen, and is sufficient to increase, improve, or boost an immune response, in the absence of traditional, non-covalently attached adjuvants.

The immunogen of the instant application has been modified either recombinantly or synthetically, such that it is capable of spontaneously forming an isopeptide bond with a binding partner. That is, the immunogen is modified with a tag, a short, unfolded peptide (“peptide tag”), that can be genetically fused to exposed positions in the immunogen such that it is capable of spontaneously forming an isopeptide bond with a binding partner on the central carrier.

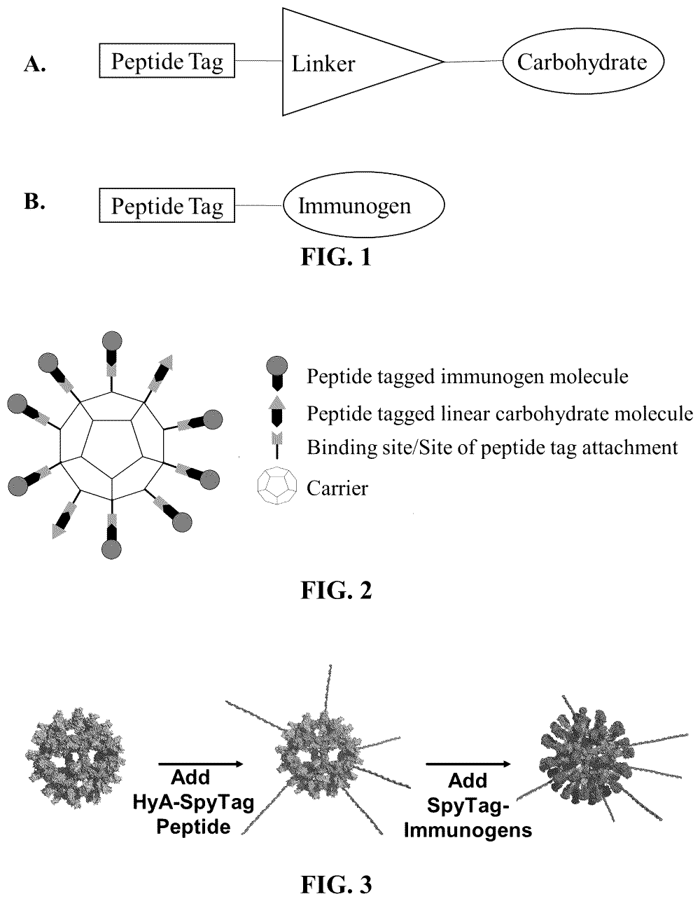

A peptide tag/binding partner pair may be derived from any protein capable of spontaneously forming an isopeptide bond (an isopeptide protein), wherein the domains of the protein are expressed or synthesized separately to produce a peptide tag that comprises one of the residues involved in the isopeptide bond (e.g. a lysine) and a peptide binding partner that comprises the other residue involved in the isopeptide bond (e.g. an asparagine or aspartate). It is important to note that although the peptide tag may be based upon a sequence of a fragment of an isopeptide protein, the sequence of the peptide tag can vary from that of the isopeptide protein or the fragment thereof. The binding partner may possess the necessary catalytic activity to form the isopeptide bond or a third protein may provide this activity. A number of peptide tag/binding partners are known in the literature (i.e., U.S. Pat. Nos. 9,547,003, 10,889,622; 10,526,379; 10,527,609; 11,059,867, GB2104999.4, WO 2022/088953, WO 2021/163438, U.S. 2022/0169681, WO 2021/250626, Brune et al., 2017, and Kou et al., 2022) and may be used with the current invention. The present invention provides a peptide tag that is modified by the covalent attachment of a linear carbohydrate at the reducing end of the carbohydrate, for use with a binding partner in which the peptide tag/binding partner pair may be derived from any protein capable of spontaneously forming an isopeptide bond. In some embodiments, an amino acid linker or other linker that is not involved in the isopeptide bond formation is present between the isopeptide bond forming sequence of the peptide tag and the site of covalent attachment of the linear carbohydrate. The peptide tag-linker-carbohydrate molecule is depicted schematically in FIG. 1A.

In one embodiment, the peptide tag has a length of at least about 5 amino acids and comprises a first reactive residue involved in formation of an isopeptide bond with a binding partner capable of forming an isopeptide bond, wherein a linear carbohydrate is covalently attached to the peptide tag. In one embodiment, the peptide tag has a length of about 5 amino acids to about 100 amino acids. In another embodiment, the peptide tag has a length of about 5 amino acids to about 50 amino acids. In a preferred embodiment, the peptide tag has a length of about 5 amino acids to about 25 amino acids.

The binding partner comprises a second reactive residue involved in the isopeptide bond formation, wherein the binding partner is at least about 20 amino acids in length, the binding partner does not include the first reactive residue of the peptide tag, and the peptide tag and the binding partner are capable of binding to each other and forming an isopeptide bond between the first and second reactive residues. One skilled in the art will appreciate that the binding of the peptide tag and the binding partner may require one or more non-covalent interactions to occur prior to isopeptide formation, and that the non-covalent interactions and the isopeptide bond formation may be mediated by the binding partner or a third peptide or protein. One skilled in the art will also recognize that while covalent attachment of the linear carbohydrate may slow isopeptide bond formation, it does not prevent the isopeptide bond from forming.

In another embodiment, when the first reactive residue of the peptide tag comprises a reactive lysine residue, the second reactive residue of the binding partner comprises a reactive asparagine, aspartic acid, glutamine, or glutamic acid residue. In another embodiment, when the first reactive residue of the peptide tag comprises a reactive asparagine, aspartic acid, glutamine, or glutamic acid residue, the second reactive residue of the binding partner comprises a reactive lysine residue or a reactive alpha-amino terminus.

One skilled in the art would recognize that more than one type of reactive moiety may exist on a peptide tag. For example, the peptide tag may contain reactive moieties needed to form an isopeptide bond and one or more reactive moieties for the covalent attachment to a linear carbohydrate molecule. In some embodiments, the peptide tag further comprises at least one reactive moiety utilized to form a covalent attachment through the reducing end of a linear carbohydrate. The location of the at least one reactive moiety is independently and individually selected from the group consisting of the C terminus, the N terminus, and internal residues of the peptide tag, wherein the at least one reactive moiety is covalently attached or bonded through the reducing end of a linear carbohydrate. In some embodiments, an amino acid linker that is not involved in the isopeptide bond formation is present between the isopeptide bond forming sequence of the carbohydrate and the at least one reactive moiety of the peptide tag. One skilled in the art would understand that covalently attached and covalently bonded are synonymous terms. One skilled in the art would also recognize that covalently attached may mean direct attachment or glycosylation at the anomeric center of the linear carbohydrate or attachment through an intermediate moiety at the anomeric center of the linear carbohydrate.

In some embodiments the at least one reactive moiety of the peptide tag comprises at least one nucleophilic amino moiety wherein the at least one nucleophilic amino moiety is covalently attached through the reducing end of a linear carbohydrate. In one embodiment, the location of the at least one nucleophilic amino moiety is independently and individually selected from the group comprising the C terminus, the N terminus, and internal residues of the peptide tag, wherein the at least one nucleophilic amino moiety is covalently attached through the reducing end of a linear carbohydrate. In another embodiment, the least one nucleophilic amino moiety is a reactive lysine residue or the N-terminal amino group. One skilled in the art will appreciate that the nucleophilic amine moiety may reside on a side chain not involved in isopeptide formation or at the N-terminus of the peptide tag. In some embodiments, there is an optional linker between the isopeptide bond forming sequence and the at least one nucleophilic amino moiety.

In one embodiment, the linear carbohydrate is functionalized at the reducing end with an oxazoline. In this embodiment, the at least one nucleophilic amino moiety covalently bonds to the oxazoline of the linear carbohydrate to form one or more amidine moieties (Schemes 1 and 2), utilizing the methodology as described in U.S. Pat. Nos. 11,021,730 and 11,643,376, incorporated by reference herein. Scheme 3 shows schematically how the amidine forms at the reducing end of a hyaluronic acid polymer after covalent conjugation to a protein, including protein immunogens of the present invention. These tautomers may be in equilibrium with each other, and other structures related to these two canonical structures, including but not limited to an imidazole ring. One skilled in the art will recognize that, as defined by IUPAC, the stereochemistry at positions 1, 2, 3, 4, and 5 of the hexose ring can be either (R) or (S) and that the carbohydrate can exist in the closed ring form or open-chain form.

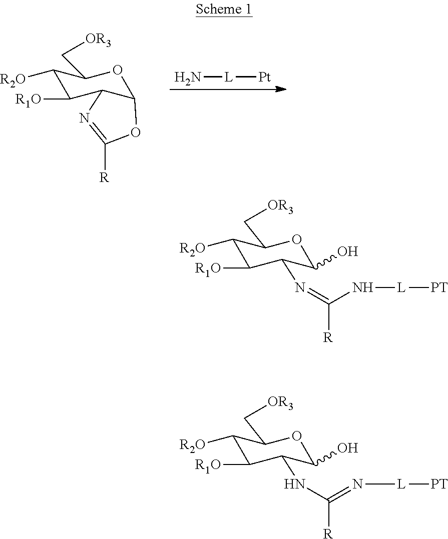

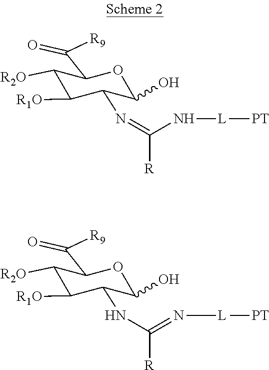

In another embodiment, the linear carbohydrate is reductively aminated using standard methods at the reducing end with a nucleophilic amino moiety of the peptide tag with an optional linker (Scheme 4). One skilled in the art will recognize that the starting carbohydrate exists in both a closed six-membered ring hemiacetal form and an aldehyde open form, and wherein the latter form reacts with amine nucleophile from the peptide tag.

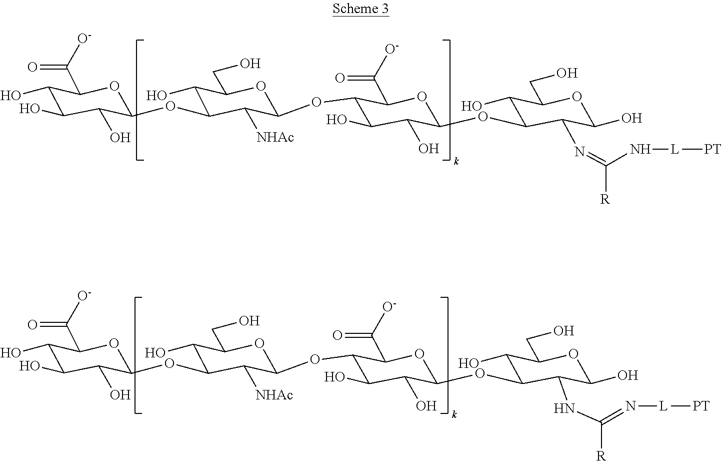

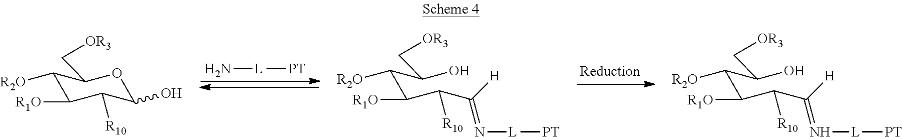

There is no stereochemical requirement at C2, C3, C4, C5 and C6 of the carbohydrate, and the N-acyl moiety may be either equatorially or axially disposed and still result in formation of an oxazoline.

One skilled in the art will recognize that the C3, C4 and C6 hydroxyl moieties on the parent N-acyl-2-amino carbohydrate may be further substituted with other carbohydrates to form larger linear oligosaccharides.

Each R is individually and independently selected from the group consisting of C₁-C₆ alkyls, branched C₃-C₈ alkyls, (CH₂)ₘ—CN, (CH₂)ₘOR₆, (CH₂)ₘ—CO₂H, (CH₂)ₘ—CO₂R₆, (CH₂)ₘ—NR₆(R₇), (CH₂)ₘ—S(O)ₙ—C₁-C₆alkyl, (CH₂)ₘ—C(O)NR₆(R₇), (CH₂)ₘ—CO₂—C₄-C₆ heterocyclyl, (CH₂)ₘ—C₄-C₆ heterocyclyl, (CH₂)ₘ—CO₂—C₄-C₆ heteroaryl, and (CH₂)ₘ—C₄-C₆-heteroaryl, wherein each alkyl may optionally contain an ether linkage and, wherein each alkyl is optionally substituted with one or two C₁-C₆ alkyls;

R₁, R₂ and R₃ are each independently selected from the group consisting of H, saccharides including but not limited to glucosamines, acetylated glucosamines, uronic acids, and polymeric carbohydrates taken from the group consisting of chitin, partially deacylated chitin, chitosan, partially acylated chitosan, hyaluronic acid, keratin, keratin sulfate, chondroitin, chondroitin sulfate, dermatan, dermatan sulfate, heparin, and derivatives thereof;
each R₆ and R₇ is individually and independently H, C1-C6 alkyl, or branched C3-C8 alkyl;
each k is individually and independently 0-1,000;
each m is individually and independently 1, 2, 3, 4, or 5;
each n is individually and independently 0, 1, or 2;
each R₈ is individually and independently H, C1-C6 alkyl, or branched C3-C8 alkyl, wherein each alkyl may optionally contain an ether linkage and, wherein each alkyl is optionally substituted with an aryl group or one or two C1-C6 alkyl;
R₉ is selected from the group consisting of OR₈, NHR₈, and NR₈R₈.
R₁₀ is selected from the group consisting of R₁, R₉, and NR₈(C═O)R R₁₁ is selected from group consisting of —(OCH₂CH₂O)ₖ—, and —O(CH₂)ₖ—, —(CH₂)ₖ—, C₁-C₆ alkyls, branched C₃-C₈ alkyls, —(CH₂)ₘOR₆—, —(CH₂)ₘ—CO₂R₆—, —(CH₂)ₘ—S(O)ₙ—C1-C6alkyl-, —(CH₂)ₘ—CO₂—C₄-C₆ heterocyclyl-, —(CH₂)ₘ—C4-C6 heterocyclyl-, —(CH₂)ₘ—CO₂—C4-C6 heteroaryl-, and —(CH₂)ₘ—C₄-C₆-heteroaryl, wherein each alkyl may optionally contain an ether linkage and, wherein each alkyl is optionally substituted with one or two C₁-C₆ alkyls and wherein each alkyl chain may optionally contain one or more sites of unsaturation;
L is an optional linker (described herein).
PT is the peptide tag (described herein).

Suitable linear carbohydrates include, but are not limited to, chitin, partially deacylated chitin, chitosan, partially acylated chitosan, hyaluronic acid, keratin, keratin sulfate, chondroitin, chondroitin sulfate, dermatan, dermatan sulfate, and heparin, and derivatives thereof. In one embodiment, the linear carbohydrate has a molecular weight of at least about 500 Daltons, at least about 1,000 Daltons, at least about 1,500 Daltons, at least about 3,500 Daltons, at least about 6,000 Daltons, at least about 10,000 Daltons, at least about 20,000 Daltons, at least about 25,000 Daltons, at least about 33,000 Daltons, at least about 50,000 Daltons, and at least about 120,000 Daltons. In another embodiment, the linear carbohydrate has a molecular weight of less than about 50,000 Daltons, or less than about 6,000 Daltons. In another embodiment, the linear carbohydrate has a molecular weight of about 10,000 Daltons to about 120,000 Daltons, about 20,000 Daltons to about 80,000 Daltons, and about 30,000 Daltons to about 50,000 Daltons. In a preferred embodiment, the linear carbohydrate has a molecular weight of about 110,000 Daltons, about 50,000 Daltons, about 40,000 Daltons, about 30,000 Daltons, about 20,000 Daltons, about 10,000 Daltons, and about 6,000 Daltons.

In another embodiment, the linear carbohydrate is hyaluronic acid (HyA), preferably with a molecular weight of between about 6 and about 75 kD. In a more preferred embodiment, the size of the hyaluronic acid is between about 10 and about 50 kD. In an embodiment, the size of the hyaluronic acid is about 110,000 Daltons, about 50,000 Daltons, about 40,000 Daltons, about 30,000 Daltons, about 20,000 Daltons, about 10,000 Daltons, and about 6,000 Daltons. In a more preferred embodiment, the size of the hyaluronic acid is about 50,000 Daltons, about 20,000 Daltons, and about 10,000 Daltons.

In one embodiment, the linear carbohydrate is endowed with adjuvant properties. In a preferred embodiment, hyaluronic acid as an adjuvant is similar or superior to already known adjuvants, for example: Aluminum (amorphous aluminum hydroxyphosphate sulfate (AAHS), Alhydrogel®, aluminum hydroxide, aluminum phosphate, Alum), Quil-A®, Addavax™, Complete Freund's Adjuvant, AS03, AS04, MF59®, ASO1_{B}, MPL®, CpG 1018, Poly I:C, Poly I:C12U, Poly I:CLC, Flagellin-based, Resiquimod-based (i.e., R848), Glucopyranosyl Lipid Adjuvant (GLA), Chitosan, LPS, Matrix-M™, Montanide ISA™51 (incomplete Freud's adjuvant), and Montanide ISA™720.

The linear carbohydrate according to the present invention is not limited by its source or origin and encompasses those obtained from natural origins including those from human and other mammalian sources, those produced by genetically engineered animal cells, plant cells, microorganisms, and other cells, those enzymatically manufactured, those manufactured by fermentation processes, those artificially synthesized by chemical processes, and others. In some embodiments, the linear carbohydrate may encompass monosaccharides, disaccharides, oligosaccharides, polysaccharides and modified derivatives thereof, so long as the reducing end saccharide moiety is unprotected on C1 (i.e., contains a hemiacetalic hydroxyl). In some embodiments, the linear carbohydrate contains an N-acyl-2-amino moiety (a 2-deoxyacetylated 2-amino moiety).

The term “modified carbohydrate” (or “modified derivative thereof”) used herein may refer to those modified through any process of isolation, separation, and purification from naturally-occurring sources and origins, those that have been enzymatically modified, those that have been chemically modified, those that have been modified by biochemical means, including microorganisms, wherein such modifications may comprise those known in the field of glycoscience, for example, alkylation, hydrolysis, oxidation, reduction, esterification, acylation, amidation, amination, etherification, nitration, dehydration, glycosylation, phosphorylation, sulfation, thiolation, alkynylation, and azidylation. In some embodiments, derivatives include those that enable “click chemistry” such as the alkynylation, azidylation, or other moieties suitable for reaction with the at least one reactive moiety of the peptide tag. One skilled in the art will appreciate that the moieties, including but not limited to hydroxyl, carboxylate, and amide, within the linear carbohydrate could be modified by these methods. In an embodiment, the modification of the carboxyl group is at least about 25%. In another embodiment, the modification of the carboxyl group is at least about at least about 50%, In a preferred embodiment, the modification of the carboxyl group is at least about 75%. In one embodiment, the carboxyl groups are esterified with an alkyl group, i.e., methyl, ethyl, propyl, dodecyl, and pentyl benzyl. In another embodiment, carboxyl groups which are not esterified with an alkyl group as above, may be reacted with lipid chain/alkyl residues from a C₁₀₋₂₀ aliphatic alcohol to produce “mixed” esters. The modified or unmodified carbohydrate is not cross-linked to hydroxyl groups of the same or different modified or unmodified carbohydrate molecule.

In some embodiments the at least one reactive moiety of the peptide tag comprises a moiety suitable for “click chemistry,” such as an alkyne or azide, with a suitably modified linear carbohydrate containing a complementary moiety to enable click chemistry such as an azide (for reaction with the peptide tag alkyne) or an alkyne (for reaction with a peptide tag azide) (for examples see Agrahari et al., 2021). In some embodiments, there is an optional linker between the at least one reactive moiety and the peptide tag. In some embodiments, there is an optional linker between the complementary moiety and the linear carbohydrate.

In one embodiment, the at least one reactive moiety comprises a terminal alkyne. The location of the terminal alkyne moiety is independently and individually selected from the group consisting of the C terminus, the N terminus, and internal residues of the peptide tag. In some embodiments, there is an optional linker between the isopeptide bond forming sequence and the terminal alkyne moiety. The terminal alkyne moiety undergoes a copper(I)-catalyzed 1,3-dipolar cycloaddition with an azide introduced at the reducing end of a linear carbohydrate as shown in Scheme 5 (Tanaka et al., 2009) and for hyaluronic acid in Scheme 6. One or more ligands may also be added to the reaction to facilitate triazole formation. The scope of all terms is defined herein.

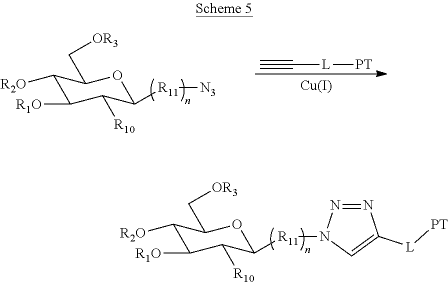

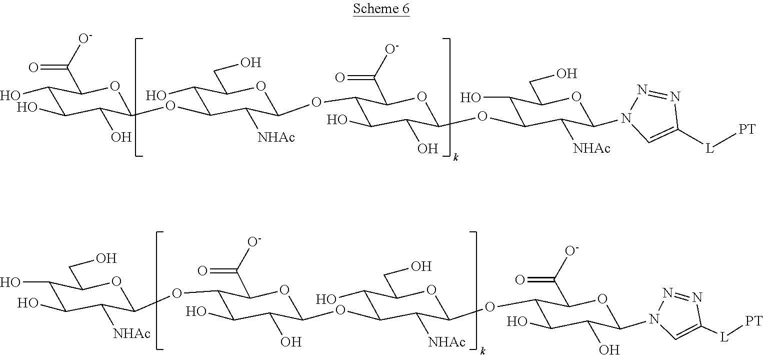

In another embodiment, the at least one reactive moiety comprises an azide. The location of the azide moiety is independently and individually selected from the group consisting of the C terminus, the N terminus, and internal residues of the peptide tag. In some embodiments, there is an optional linker between the isopeptide bond forming sequence and the azide moiety. The azide moiety undergoes a copper(I)-catalyzed 1,3-dipolar cycloaddition with a terminal alkyne introduced at the reducing end of a linear carbohydrate (see Upadhyay et al., 2009) as shown in Scheme 7.

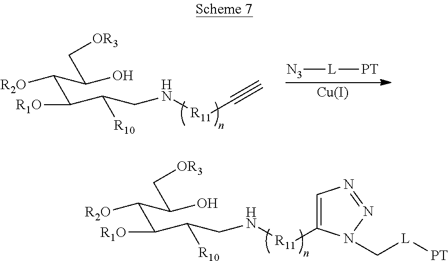

In one embodiment, the at least one reactive moiety comprises an azide. The location of the azide moiety is independently and individually selected from the group consisting of the C terminus, the N terminus, and internal residues of the peptide tag. In some embodiments, there is an optional linker between the isopeptide bond forming sequence and the azide moiety. The azide moiety undergoes a copper(I)-catalyzed 1,3-dipolar cycloaddition with a terminal alkyne introduced at the reducing end of a linear carbohydrate as shown in Scheme 8 and for hyaluronic acid in Scheme 9. One or more ligands may also be added to the reaction to facilitate triazole formation.

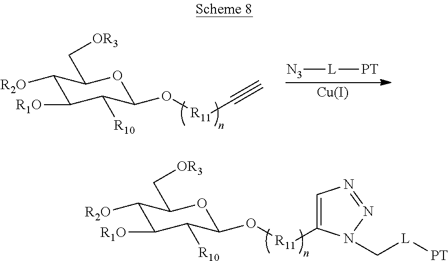

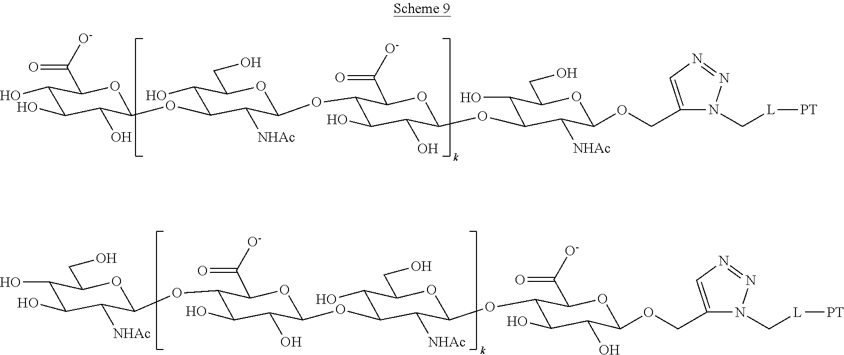

In some embodiments, the at least one reactive moiety comprises a terminal alkene. The location of the terminal alkene moiety is independently and individually selected from the group consisting of the C terminus, the N terminus, and internal residues of the peptide tag. In some embodiments, there is an optional linker between the isopeptide bond forming sequence and the terminal alkene moiety. The terminal alkene moiety undergoes a Michael reaction with a thiol introduced at the reducing end of a linear carbohydrate (Köhling et al., 2016) as shown in Scheme 10.

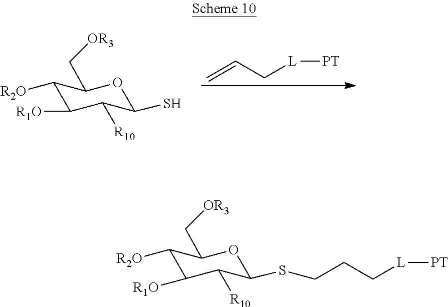

In another embodiment, the at least one reactive moiety comprises a terminal thiol moiety (for example a cysteine). The location of the terminal thiol moiety is independently and individually selected from the group consisting of the C terminus, the N terminus, and internal residues of the peptide tag. In some embodiments, there is an optional linker between the isopeptide bond forming sequence and the terminal thiol moiety. The terminal thiol moiety undergoes a Michael reaction with an alkenyl group introduced at the reducing end of a linear carbohydrate as shown in Scheme 11.

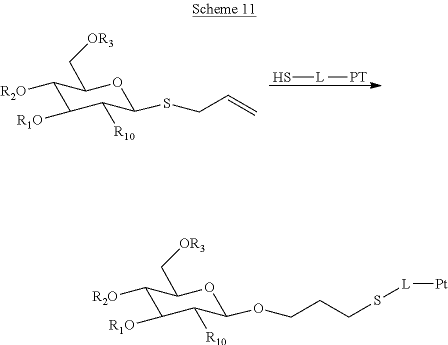

A variety of synthetic methods and moieties may be utilized to form a covalent bond between the reducing end of a linear carbohydrate and the at least one reactive moiety of the peptide tag.

Any nonreactive linker may be used. The optional linker is nonreactive residues or moieties that would be recognized by one skilled in the art. In one embodiment, the optional linker is composed of nonreactive residues that provide a spacer between the isopeptide bond forming sequence and the at least one reactive moiety. In some embodiments, an amino acid linker that is not involved in the isopeptide bond formation is present between the isopeptide bond forming sequence and the site of covalent attachment of the linear carbohydrate. In some embodiments, the linker comprises glycine (G) and/or serine (S) amino acids. For example, one linker is a glycine chain of from about 1 to about 20 residues. In some embodiments, the linker is a glycine-serine linker. For example, the glycine-serine linker could be glycine-glycine, serine-serine, glycine-serine, serine-glycine combined and repeated up to ten times. In some embodiments, the linker is about 2-4, 2-6, 2-8, 2-10, 2-12, or 2-14 amino acids in length. In another embodiment, the linker is at least 15 amino acids in length. In another embodiment, the linker is at least 25 amino acids in length. One skilled in the art would appreciate that the linker sequence can be optimized by iterative variation of the linker sequence to maximize the experimentally determined immunogenicity of the construct.

As used herein, the terms “antigen” or “immunogen” are used interchangeably to refer to a substance, typically a peptide or a protein, which is capable of inducing an immune response in a subject (e.g., a mammal, such as a human). The term also refers to peptides and proteins and derivatives thereof that are immunologically active in the sense that once administered to a subject, are capable of or intended to evoke an immune response of the humoral and/or cellular type directed against that protein or peptide or a variant thereof. As discussed above, the immunogen of the instant application has been modified either recombinantly or synthetically, such that it is capable of spontaneously forming an isopeptide bond with a binding partner. The immunogens of the current invention can be recombinant or synthetic. Immunogens for use in vaccines can be identified by a variety of methods known to one of skill in the art (e.g., Earnhart et al., 2007; Olsen et al., 2015; He et al. 2021). The position of the isopeptide bond forming sequence on the immunogen may be located at the N-terminus, the C-terminus, or internal residues of the recombinant or synthetic immunogen (see, for example, U.S. Pat. Pub. No. 2022/0119459 or Khairil Anuar et al., 2019).

The immunogen may already be N-glycosylated or O-glycosylated, thereby providing a hyper-glycosylated immunogen. An immunogen may be post-transitionally modified either by natural or synthetic means. Post-translational modification (PTM) is the chemical modification of a protein after its translation and involves the later steps in protein biosynthesis for many proteins. One or more PTMs may occur on the same immunogen. PTMs can occur in vivo (natural) or in vitro (synthetic). A list of natural PTMs includes, but is not limited to, ADP-ribosylation, glycosylation, glypiation, isoprenylation, methylation, myristoylation, oxidation, sulfation, palmitoylation, phosphorylation, prenylation, and polysialylation. A list of synthetic PTMs includes, but is not limited to, amidation, biotinylation, glycation, methylation, oxidation, pegylation, phosphorylation, reductive amination, and sulfation.

In a typical embodiment, the peptide-tagged linear carbohydrate molecules are added to the nanoparticle carrier at the desired loading of the linear carbohydrate to create an isopeptide bond between the peptide tag on the carbohydrate and the binding partner on the carrier to yield a central carrier complex onto which are covalently bound a desired amount of linear carbohydrate molecules. There are remaining binding partners presenting that are still available as binding sites to further load the immunogen. Thus, once the carbohydrate is loaded, a peptide-tagged immunogen can be added to the nanoparticle carrier to create an isopeptide bond between the peptide tag on the immunogen and the remaining binding partners on the carrier to create the fully formed nanoparticle carrier complex. This sequence is depicted in FIG. 3.

The number of linear carbohydrate molecules/binding partner pairs and immunogen/binding partner pairs can be the same or different on the carrier. In some embodiments, the peptide-tagged linear carbohydrate molecules are added to the nanoparticle carrier at the desired loading of the linear carbohydrate to create an isopeptide bond between the peptide tag on the carbohydrate and the binding partner on the carrier to yield a central carrier complex onto which are covalently bound a desired amount of linear carbohydrate molecules. This complex can then be isolated, purified if necessary, and provided as part of a kit for subsequent addition of a desired immunogen to the remaining available binding partners on the carrier. In some embodiments, the linear carbohydrate is individually and independently selected from the group consisting of hyaluronic acid and chitosan. In a more preferred embodiment, the linear carbohydrate is individually and independently selected from the group consisting of HyA(6K), HyA(12K), HyA(25K), HyA(33K), HyA(50K), chitosan(6K), chitosan(12K), chitosan(25K), chitosan(33K), chitosan(50K), chitosan(100K), and chitosan(200K).

A pathogen is an agent that causes disease or illness in a host (i.e., human), including but not limited to, bacteria, fungi, viruses, helminths, and protozoa.

In an embodiment, the immunogens of the instant application are derived from pathogens. In another embodiment, the immunogens are derived from pathogens selected from, but not limited to, protozoa, fungi, helminths, bacteria, and viruses. In a preferred embodiment, the immunogen is derived from a viral or bacterial pathogen. In a more preferred embodiment, the immunogen is derived from a viral pathogen (virus). Immunogens for use in vaccines can be identified by a variety of methods.

In another embodiment, the protozoan pathogens are selected from the group consisting of, but not limited to, the genera Acanthamoeba, Ixodes, Entamoeba, Giardia, Naegleria, Plasmodia, Toxoplasma, Trichomonas, and Trypanosoma.

In another embodiment, the helminthic pathogens are selected from the group consisting of, but not limited to, the genera Ancylostoma, Ascaris, Clonorchis, Diphyllobothrium, Echinococcus, Echinocococcus, Enterobius, Fasciola, Necator, Onchocerca, Schistosoma, Strongyloides, Taenia, Trichuris, and Wuchereria,

In another embodiment, the fungal pathogens are selected from the group consisting of, but not limited to, the genera Arthrodermataceae, Aspergillus, Basidiobolus, Blastomyces, Candida, Coccidioides, Conidiobolus, Cryptococcus, Epidermophyton, Eumycetoma, Fusarium, Histoplasma, Lacazia, Malassezia, Microsporum, Mucormycetes, Paracoccidioides, Pneumocystis, Pseudoallescheria, Rhizopus, Sporothrix, Stachybotrys, Talaromyces, Trichophyton, and Trichosporon.

In another embodiment, the bacterial pathogens are selected from the group consisting of, but not limited to, the genera Achromobacter, Acanthocheilonema, Acinetobacter, Aeromonas, Anaplasma, Babesia, Bacillus, Bacteroides, Bartonella, Borrelia, Bordetella, Brucella, Burkholderia, Campylobacter, Capnocytophaga, Chlamydia, Citrobacter Clostridium, Corynebacterium, Coxiella, Ehrlichia, Eikenella, Enterococcus, Escherichia, Francisella, Fusobacterium, Gardnerella, Haemophilus, Helicobacter, Klebsiella, Lactobacillus, Legionella, Leptospira, Listeria, Moraxella, Morganella, Mycobacterium, Mycoplasma, Neisseria, Nocardia, Pasteurella, Peptostreptococcus, Porphyromonas, Propionibacterium, Proteus, Providencia, Pseudomonas, Rickettsia, Salmonella, Schistosoma, Serratia, Shigella, Staphylococcus, Streptococcus, Treponema, Ureaplasma, Vibrio, and Yersinia.

In another embodiment, the viral pathogens are selected from the group consisting of, but not limited to, the genera Alphacoronavirus, Alphapapillomavirus, Alphatorquevirus, Alphavirus, Arenavirus, Bornavirus, Betacoronavirus, Betapapillomavirus, Cardiovirus, Coltvirus, Cosavirus, Cytomegalovirus, Deltaretrovirus, Deltavirus, Dependovirus, Dependoparvovirus, Ebolavirus, Enterovirus, Erythrovirus, Flavivirus, Gammapapillomavirus, Hantavirus, Henipavirus, Hepacivirus, Hepatovirus, Hepevirus, Influenzavirus A, Influenzavirus B, Influenzavirus C, Jeilongvirus, Kobuvirus, Lentivirus, including human immunodeficiency virus, Lymphocryptovirus, Lyssavirus, Mamastrovirus, Marburgvirus, Mastadenovirus, Molluscipoxvirus, Morbillivirus, Mupapillomavirus, Nairovirus, Norovirus, Nupapillomavirus, Orthobunyavirus, Orthohepadnavirus, Orthohepevirus, Orthopneumovirus, Orthopoxvirus, Parapoxvirus, Parechovirus, Pegivirus, Phlebovirus, Picobirnavirus, Polyomavirus, Posavirus, Respirovirus, Rhadinovirus, Rhinovirus, Rosavirus, Roseolovirus, Rotavirus, Rubivirus, Rubulavirus, Salivirus, Sapovirus, Seadornavirus, Simplexvirus, Spumavirus, Thogotovirus, Torovirus, Varicellovirus, and Vesiculovirus.

In an embodiment the immunogens are derived from pathogens selected independently and individually from the group consisting of Influenzavirus A, Influenzavirus B, Influenzavirus C, Rhinovirus, Lentivirus, including human immunodeficiency virus, Respirovirus including respiratory syncytial virus (RSV), Orthopneumovirus including human Orthopneumovirus, Alphacoronavirus, Betacoronavirus including Middle East respiratory syndrome-related coronavirus (MERS-CoV), SARS-CoV, and COVID-19, Flavivirus including dengue viruses, Zika virus, and West Nile virus, Hepatovirus including hepatitis A, B, C, D, and E, Norovirus, Marburgvirus including Marburg virus, Orthopoxvirus, Togaviridae, Ebolavirus including Ebola virus, Babesia, Borrelia, Staphylococcus including methicillin-resistant Staphylococcus aureus (MRSA), Legionella, Chlamydia, Plasmodia, Streptococcus pneumoniae, Vibrio cholerae, Listeria, Clostridia, Salmonella, Bordetella, Enterococci, Treponemia, Amoeba, Neisseria, and Giardia.

One skilled in the art will appreciate that for each pathogen listed, one or more serotypes may be relevant to a particular disease state caused by the pathogen, including serotypes not yet identified.

In an embodiment, the immunogens are derived from Betacoronavirus, more specifically SARS-related emergent zoonotic coronaviruses. In a preferred embodiment, the immunogens are derived from SARS-CoV, MERS-CoV, and SARS-CoV-2 (COVID-19). In some embodiments, the vaccine composition comprises one or more immunogens from the same pathogen genus. That is, the vaccine composition comprises two, three, four, or more different from one another immunogens from the same pathogen genus.

A carrier as used herein can be generally referred to as a biocompatible molecular system having the capability to present multiple binding partners, and of incorporating and transporting molecules (e.g., therapeutic agents such as immunogens) to enhance their selectivity, bioavailability, and efficiency. One of ordinary skill in the art would also refer to a carrier as a scaffold. The carrier can present at least 1, at least 2, at least 10, at least 20, at least 40, at least 60, or any number in between 1 to 60 binding partners for attachment of respective linear carbohydrate or immunogen molecules. The carriers used in the methods, compositions, and systems herein described can be a biocompatible molecular system, either naturally occurring or synthetic, that can be functionalized or conjugated for coupling (e.g., covalently) to a plurality of peptide tags covalently attached to linear carbohydrate molecules and/or a plurality of immunogen molecules described herein. The carriers can comprise nanoparticles, nanotubes, nanowires, dendrimers, liposomes, ethosomes and aquasomes, polymersomes and niosomes, foams, hydrogels, cubosomes, quantum dots, exosomes, macrophages, and others identifiable to a person skilled in the art.

In some embodiments, the carrier used herein can be a nanosized carrier such as a nanoparticle. As used herein, the term “nanoparticle” can refer to a nanoscopic particle having a size measured in nanometers (nm). Size of the nanoparticles may be characterized by their maximal dimension. The term “maximal dimension” as used herein can refer to the maximal length of a straight-line segment passing through the center of a nanoparticle and terminating at the periphery. In the case of nanospheres, the maximal dimension of a nanosphere corresponds to its diameter. The term “mean maximal dimension” can refer to an average or mean maximal dimension of the nanoparticles and may be calculated by dividing the sum of the maximal dimension of each nanoparticle by the total number of nanoparticles. Accordingly, value of maximal dimension may be calculated for nanoparticles of any shape, such as nanoparticles having a regular shape such as a sphere, a hemispherical, a cube, a prism, or a diamond, or an irregular shape. The nanoparticles provided herein need not be spherical and can comprise, for example, a shape such as a cube, cylinder, tube, block, film, and/or sheet. In some embodiments, the maximal dimension of the nanoparticles is in the range from about 1 nm to about 5,000 nm, such as between about 20 nm to about 1,000 nm, about 20 nm to about 500 nm, about 20 nm to about 300 nm, about 20 nm to about 100 nm, or about 20 to about 50 nm.

The nanoparticle can be, but is not limited to, any one of lipid-based nanoparticles (nanoparticles where the majority of the material that makes up their structure are lipids, e.g., liposomes or lipid vesicles), polymeric nanoparticles, inorganic nanoparticles (e.g., magnetic, ceramic, and metallic nanoparticles), surfactant-based emulsions, silica nanoparticles, virus-like particles (particles primarily made up of viral structural proteins that are not infectious or have low infectivity), peptide or protein-based particles (particles where the majority of the material that makes up their structure are peptides or proteins) and/or nanoparticles that are developed using a combination of nanomaterials such as lipid-polymer hybrid nanoparticles formed by polymer cores and lipid shells or nano lipoprotein particles formed by a membrane forming lipid arranged in a membrane lipid bilayer stabilized by a scaffold protein as will be understood by a person skilled in the art.

In some embodiments, a carrier is made up of a plurality of monomeric subunits which assemble with one another through covalent and/or non-covalent forces to form the carrier. In some embodiments, the carrier described herein is a protein nanoparticle comprising a plurality of particle-forming proteins, which are the monomeric subunit proteins that form the protein nanoparticle. Protein nanoparticles can be categorized into non-viral protein nanoparticles and viral-like particles (VLPs). Examples of non-viral protein nanoparticles include, but are not limited to, ferritins, vaults, heat-shock proteins, chaperonins, lumazine synthase, encapsulins, and bacterial microcompartments. VLPs can be derived from viruses including, but not limited to, adenovirus, cowpea mosaic virus, cowpea chlorotic mottle virus, brome mosaic virus, broad bean mottle virus, bacteriophage lambda (e.g., bacteriophage lambda procapsid), MS2 bacteriophage, Qβ bacteriophage, P22 phage capsid, and others identifiable to a person skilled in the art.

In some embodiments, VLP refers to a non-replicating, viral shell, derived from any of several viruses. VLPs can be naturally occurring or synthesized through the individual expression of viral structural proteins, which can then self-assemble into the virus-like structure. VLPs are generally composed of one or more viral proteins, such as particle-forming proteins referred to as capsid, coat, shell, surface and/or envelope proteins, or particle-forming polypeptides derived from these proteins. In some embodiments, VLPs can form spontaneously upon recombinant expression of the protein in an appropriate expression system. VLPs can differ in morphology, size, and number of subunits. Methods for producing VLPs are known in the art. The presence of VLPs following recombinant expression of viral proteins can be detected using conventional techniques also known in the art, such as by electron microscopy, biophysical characterization, and the like (see e.g., Baker et al., 1991, Hagensee et al., 1994, and Mejía-Méndez et al, 2022). For example, VLPs can be isolated by density gradient centrifugation and/or identified by characteristic density banding. Alternatively, cryoelectron microscopy can be performed on vitrified aqueous samples of the VLP preparation in question, and images recorded under appropriate exposure conditions. Any of a variety of VLPs known in the art can be used herein, including but not limited to, Aquifex aeolicus lumazine synthase, Thermotoga maritima encapsulin, Myxococcusanthus encapsulin, bacteriophage Qbeta virus particle, Flock House Virus (FHV) particle, ORSAY virus particle, and infectious bursal disease virus (IBDV) particle. In some embodiments, the nanoparticle used herein can be a bacteriophage VLP, such as Ap205 VLP. In some embodiments, the nanoparticle used herein is a mutated Ap205 VLP (for example, SpyCatcher-CP3, Brune et al., 2016).

In some embodiments, the nanoparticles described herein comprise a self-assembling nanoparticle. A self-assembling nanoparticle typically refers to a ball-shape protein shell with a diameter of tens of nanometers and well-defined surface geometry that is formed by identical copies of a non-viral protein capable of automatically assembling into a nanoparticle with a similar appearance to VLPs. Examples of self-assembling nanoparticle particle-forming proteins include, but are not limited, to ferritin (FR) (e.g., Helicobacter pylori ferritin, see Zhang et al., 2020; Kang et al., 2021), which is conserved across species and forms a 24-mer, as well as B. stearothermophilus dihydrolipoyl acyltransferase (E2p, see He et al., 2021), Aquifex aeolicus lumazine synthase (LuS, see Zhang et al., 2020), and Thermotoga maritima encapsuling (see Hsia et al., 2016; Bruun et al., 2018), which all form 60-mers. In some embodiments, the self-assembling nanoparticles comprise a plurality of particle-forming proteins of 2-keto-3-deoxy-phosphogluconate (KDPG) aldolase from the Entner-Doudoroff pathway of the hyperthermophilic bacterium Theremotoga Maritima or a variant thereof. In some embodiments, mutations are introduced to the KDPG aldolase for improved particle yields, stability, and uniformity. For example, in some embodiments, mutations can be introduced to alter the interface between the wild-type protein trimer of KDPG aldolase. In some embodiments, the nanoparticle used herein is an i301 nanoparticle or a variant thereof (i.e., Hsia et al., 2016). In some embodiments, the nanoparticle used herein is a mutated i301 nanoparticle (for example, mi3 nanoparticle, Bruun et al., 2018). The self-assembling nanoparticles can form spontaneously upon recombinant expression of the protein in an appropriate expression system. Methods for nanoparticle production, detection, and characterization can be conducted using the same techniques developed for virus-like particles (VLPs).

In some embodiments, the nanoparticles described within comprise an engineered protein complex. An engineered protein complex utilizes rational or computational design to assemble dimeric, trimeric, tetrameric, or pentameric proteins (building blocks) into larger, highly oligomeric complexes. The geometric symmetry and shape of the nanoparticle is determined by the type of building blocks used (for examples see Nguyen et al., 2021). An example of an engineered protein complex includes but is not limited to I53-50 (see Kang et al, 2021).

It is understood that in any of the embodiments disclosed within that the linear carbohydrate molecule is covalently attached to a nanoparticle carrier through a peptide tag/binding partner pairing. As discussed within, the peptide tag is covalently attached to the linear carbohydrate molecule with an optional linker.

The term “present” as used herein with reference to a compound (e.g., an immunogen) or, the functional group indicates attachment performed to maintain the chemical reactivity of the compound or functional group attached. Accordingly, a functional group presented on a carrier is able to perform under the appropriate conditions the one or more chemical reactions that chemically characterize the functional group. A compound presented on a carrier is able to perform under the appropriate conditions the one or more chemical reactions that chemically characterize the compound. For example, where the compound is, or comprises, an immunogen, the immunogen presented by a carrier maintains the complex of reactions that are associated with the immunological activity characterizing the immunogen. Accordingly, presentation of an immunogen indicates an attachment such that the immunological activity associated with the immunogen attached is maintained.

In some embodiments herein described, the carriers used are multivalent carriers. Multivalent carriers can also be referred to as mosaic carriers. As opposed to a monovalent carrier which presents a single species of an immunogen or linear carbohydrate molecule, a multivalent nanoparticle carrier presents a heterologous population of bound moieties such as any combination of linear carbohydrate molecules and optionally immunogens. In one embodiment, the multivalent carrier presents one or more linear carbohydrate molecules and one or more homologous immunogens bound thereto. In another embodiment, the immunogens presented by the multivalent carrier comprise at least two heterologous immunogens of or derived from different genera, species, or strains selected from the group consisting of, but not limited to, protozoa, fungi, helminths, bacteria, and viruses. In preferred embodiment, the immunogens are derived from viral or bacterial pathogens. In a more preferred embodiment, the immunogens are derived from viral pathogens. In another more preferred embodiment, the immunogens are derived from bacterial pathogens.

The term “heterologous immunogens” means that the immunogens are of different origins, such as derived from pathogens of different taxonomic groups such as different strains, species, subgenera, genera, subfamilies or families and/or from antigenically divergent pathogens (e.g., variants thereof). Heterogeneous immunogens may be derived from pathogens of the same or different genera. Accordingly, heterologous immunogens presented on a multivalent carrier herein described have different protein sequences.

The heterologous immunogens presented on the multivalent carrier herein described can be displayed on its surface. Alternatively, the heterologous immunogens presented on the multivalent carrier herein described can be partially encapsulated or embedded such that at least an immunogenic portion of the immunogen is exposed and accessible by a host cell receptor so as to induce an immune response.

In an embodiment, the present invention provides a method for producing a self-assembling multivalent nanoparticle complex in which the self-assembling nanoparticle carrier is formed from a binding partner, and one or more linear carbohydrate molecules and immunogen molecules each bound via respective peptide tag/binding partner pairs to provide a vaccine composition. The one or more linear carbohydrate molecules and plurality of immunogen molecules can be displayed on the surface of the multivalent carrier, or partially (e.g., substantially) embedded in the multivalent carrier.

In some embodiments, the present invention provides a method for improving the solubility, the stability, or both, of the self-assembled nanoparticle complex with one or more linear carbohydrate molecules, and a plurality of immunogen molecules, each covalently bound through a peptide tag/binding partner pair to provide a vaccine composition. The solubility, the stability, or both can be modulated by the number of covalently bound linear carbohydrate molecules used as part of the nanoparticle composition.

The number of linear carbohydrate molecules and immunogen molecules each covalently bound through a peptide tag/binding partner pair can be the same or different on the carrier. In some embodiments, the number of linear carbohydrate molecules each covalently bound through a peptide tag/binding partner pair on a carrier is at least one. In another embodiment, the number of linear carbohydrate molecules is at least two, at least three, at least four, at least five, up to the total number of binding sites on the nanoparticle carrier (for example, Thermotoga maritima has 60 binding sites for up to 60 binding partner/peptide pairs). The linear carbohydrate molecules may be the same or different.

In another embodiment, the number of immunogen molecules each covalently bound through a peptide tag/binding partner pair on a central carrier is at least one. In another embodiment, the number of immunogen molecules is at least two, at least three, at least four, at least five, up to the total number of available binding sites on the central nanoparticle carrier not already covalently bound to a linear carbohydrate molecule (for example, Thermotoga maritima has 60 binding sites for up to 60 binding partner/peptide pairs). The immunogen molecules may be the same or different.

The number of linear carbohydrate molecules and immunogen molecules each covalently bound through a peptide tag/binding partner pair can be varied to determine a ratio of linear carbohydrate molecules and immunogen molecules on a given central carrier. For example, the ratio of linear carbohydrate molecules to immunogen molecules on a given central carrier can be from 1:100 to 100:1. In some embodiments, the ratio can be, be about, be at least, be at least about, be at most, be at most about, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, or a number or a range between any two of the values. In some embodiments, the ratio of immunogen molecules to linear carbohydrate molecules on a given central carrier can be, be about, be at least, be at least about, be at most, be at most about, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, or a number or a range between any two of the values. In a preferred embodiment, the ratio of linear carbohydrate molecules to immunogen molecules is about 1:60, about 1:45, about 1:30, about 1:20, and 1:12.

In one embodiment, the present invention provides a method for producing a self-assembling nanoparticle carrier in which the self-assembling nanoparticle carrier is formed from a binding partner presented on a central carrier and one or more peptide tags covalently attached to linear carbohydrate molecules in which the linear carbohydrate molecules may be the same or different.

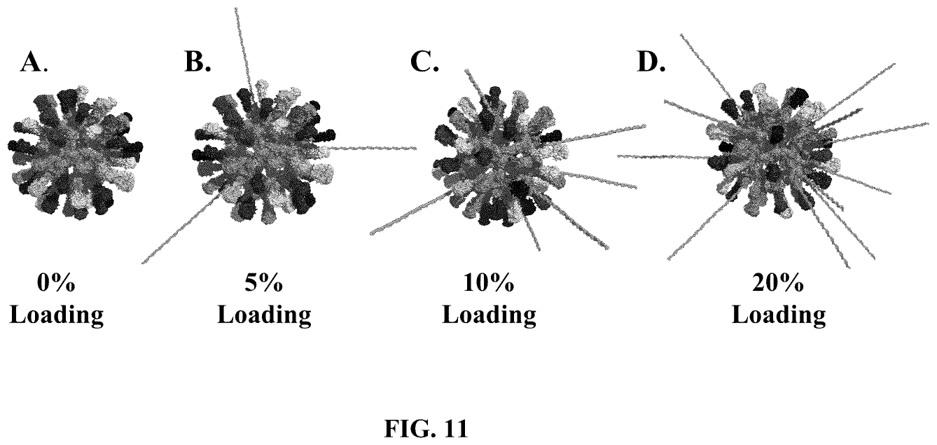

In an embodiment, the linear carbohydrate molecules and the optional immunogen molecules are covalently attached to the particle-forming proteins of the carrier (e.g., particle-forming proteins of the multivalent carrier) through a SpyTag/SpyCatcher binding pair. See FIG. 1 and FIG. 2 for schematic depictions. The SpyTag/SpyCatcher binding pair refers to a protein ligation system that is based on the internal isopeptide bond of the CnaB2 domain of FbaB from Streptococcus pyogenes (see, e.g., Zakeri et al., 2012, Keeble et al., 2019). CnaB2 is split and engineered into two complementary fragments, such that the first fragment (SpyCatcher) is able to bind and form a covalent isopeptide bond with the second fragment (SpyTag) through the side chains of a lysine in SpyCatcher and an aspartate in SpyTag. Multivalent carrier complexes can be generated to present one or more covalently attached linear carbohydrate molecules and optionally, one or more covalently attached immunogen molecules as a result of SpyTag/SpyCatcher mediated conjugation of the peptide tag covalently attached to the linear carbohydrate molecules and the immunogen molecules. See FIG. 11. The SpyTag sequence can be referred to as st003, and the SpyCatcher sequence can be referred to as sc003.

In some embodiments, the particle-forming protein of the multivalent carrier is a fusion protein containing amino acid sequences from at least two unrelated proteins that have been joined together, genetically, to express as a single protein. For example, the SpyTag motif can be independently and individually fused to the immunogenic protein and a sequence containing a nucleophilic moiety for covalent attachment of a linear carbohydrate. In this same example, the carrier subunit sequence can be fused to a SpyCatcher motif. Alternatively, the SpyCatcher motif can be independently and individually fused to the immunogenic protein and a sequence containing a nucleophilic moiety for covalent attachment of a linear carbohydrate. In this same example, the carrier subunit sequence can be fused to a SpyTag motif.

In one embodiment, the SpyTag motif can be fused C-terminal to the sequence containing a reactive moiety for covalent attachment of a linear carbohydrate and the immunogenic protein. In another embodiment, the SpyTag motif can be fused N-terminal to the sequence containing a reactive moiety for covalent attachment of a linear carbohydrate and the immunogenic protein. In one embodiment, the SpyTag motif can be fused C-terminal to the reactive moiety for covalent attachment of a linear carbohydrate and N-terminal to the immunogenic protein. In one embodiment, the SpyTag motif can be fused N-terminal to the reactive moiety for covalent attachment of a linear carbohydrate and C-terminal to the immunogenic protein. One skilled in the art would recognize that the same sequence relationships between the reactive moiety and the immunogenic protein relative to the SpyTag motif can occur not only at the termini but as part of an internal sequence of a larger construct.

In one embodiment, the SpyCatcher motif can be fused C-terminal to the carrier subunit sequence. In another embodiment, the SpyCatcher motif can be fused N-terminal to the carrier subunit sequence. One skilled in the art would recognize that the same sequence relationships between the carrier subunit sequence and the SpyCatcher motif can occur not only at the termini but as part of an internal sequence of a larger construct.

In one embodiment, the SpyCatcher motif can be fused C-terminal to the sequence containing a reactive moiety for covalent attachment of a linear carbohydrate and the immunogenic protein. In another embodiment, the SpyCatcher motif can be fused N-terminal to the sequence containing a reactive moiety for covalent attachment of a linear carbohydrate and the immunogenic protein. In one embodiment, the SpyCatcher motif can be fused C-terminal to the reactive moiety for covalent attachment of a linear carbohydrate and N-terminal to the immunogenic protein. In one embodiment, the SpyCatcher motif can be fused N-terminal to the reactive moiety for covalent attachment of a linear carbohydrate and C-terminal to the immunogenic protein. One skilled in the art would recognize that the same sequence relationships between the reactive moiety and the immunogenic protein relative to the SpyCatcher motif can occur not only at the termini but as part of an internal sequence of a larger construct.

In one embodiment, the SpyTag motif can be fused C-terminal to the carrier subunit sequence. In another embodiment, the SpyTag motif can be fused N-terminal to the carrier subunit sequence. One skilled in the art would recognize that the same sequence relationships between the carrier subunit sequence and the SpyTag motif can occur not only at the termini but as part of an internal sequence of a larger construct.

In some embodiments, the particle-forming protein can be a fusion protein containing a mi3 monomeric subunit protein at the C-terminal of the particle-forming protein and a SpyCatcher protein at the N-terminal of the particle-forming protein or a fusion protein containing a AP205-CP3 monomeric subunit protein at the C-terminal of the particle-forming protein and a SpyCatcher protein at the N-terminal of the particle forming protein such that the SpyCatcher proteins are presented or displayed for binding to the SpyTag of the immunogenic protein and linear carbohydrate.

It will be appreciated that the nanoparticle carrier can be configured to present immunogens in a number of variations, combinations, or permutations. For example, the plurality of immunogens presented by a nanoparticle carrier can be individually the same or different, the number of any particular type of immunogens presented by a nanoparticle carrier can vary, the total number of immunogens presented by a nanoparticle carrier can vary, and the ratio of any two or more different immunogen molecules can vary in different embodiments. It is understood that in each of these embodiments, whether or not specifically stated, the nanoparticle carrier presents one or more linear carbohydrate molecules covalently attached via respective peptide tags to the nanoparticle carrier.

It will be appreciated that the nanoparticle carrier can be configured to present the one or more linear carbohydrate molecules in a number of variations, combinations, or permutations. For example, the one or more linear carbohydrate molecules presented by a nanoparticle carrier can be individually the same or different, the number of any particular type of the one or more linear carbohydrate molecules presented by a nanoparticle carrier can vary, the total number of the one or more linear carbohydrate molecules presented by a nanoparticle carrier can vary, and the ratio of any two or more different linear carbohydrate molecules can vary in different embodiments.

The plurality of immunogen molecules may be the same or different. For example, the plurality of immunogen molecules may comprise a first immunogen and a second immunogen that is different from the first immunogen. In some embodiments, the plurality of immunogen molecules can comprise three, four, five, six, seven, eight, nine, ten, eleven, twelve or more different immunogens.

In one embodiment, one immunogen is considered different from another immunogen when the two immunogens are from different taxonomic groups, including from different strains, species, subgenera, genera, or subfamilies in the immunogen family. In another embodiment, one immunogen is also considered different from another immunogen when the two immunogens are derived from antigenically divergent pathogens. The term “antigenically divergent pathogen” refers to a strain of a pathogen that has a tendency to mutate or has developed mutations over time, thus changing the amino acids that are displayed to the immune system. Such mutation over time can also be referred to as “antigenic drift”.

In some embodiments, the plurality of immunogens is from different genera within a family. In some embodiments, the plurality of immunogens is from different subgenera within the same family. In some embodiments, the plurality of immunogens is from different species within the same family. In some embodiments, the plurality of immunogens contains different strains within the same family. In some embodiments, the plurality of immunogens is individually derived from different mutations, variants, or strains of a particular pathogen.

For example, the plurality of coronavirus immunogens can be of coronaviruses in the genus of Alphacoronavirus and/or Betacoronavirus. In some embodiments, the plurality of coronavirus immunogens is derived from coronaviruses in the genus of Betacoronavirus. In some embodiments, the plurality of coronavirus immunogens is derived from coronaviruses in the subgenus of Sarbecovirus. In some embodiments, the first coronavirus immunogen and the second coronavirus immunogen are derived from the genus of Betacoronavirus, optionally in the subgenus of Sarbecovirus. For example, the plurality of coronavirus immunogens can be derived from coronaviruses selected from the group consisting of SARS, SARS-2, WIV1, SHC014, Rf1, RmYNO2, pangl7, RaTG13, and Rs4081. For example, the first coronavirus immunogen, the second coronavirus immunogen, or both can be derived from the group consisting of SARS, SARS-2, WIV1, SHC014, Rf1, RmYNO2, pang17, RaTG13, and Rs4081.

The plurality of immunogen molecules individually attached to a multivalent carrier can be derived from the same protein type or corresponding proteins. One skilled in the art would appreciate that immunogen molecules of a same protein type may or may not have identical amino acid sequences, but generally share some sequence homology. In some embodiments, proteins of different immunogen taxonomic groups having the same function are considered the same protein type or corresponding proteins. In some embodiments, immunogens of a same protein type have at least 50% sequence identity, for example at least 65%, 70%, 80%, 90%, 95%, 98%, 99%, or more sequence identity. Alternatively, in some embodiments the immunogens can comprise immunogen proteins of different protein types. One skilled in the art would appreciate that immunogen proteins of different protein types typically have different functions.

The total number of homologous or heterologous immunogens presented by a multivalent carrier can be different in different embodiments. In some embodiments, the multivalent carrier herein described can present about, at least, at least about, at most, or at most about 1, 2, 3, 4, 5, 6, 7, 8, 9, 10, 11, 12, 13, 14, 15, 16, 17, 18, 19, 20, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values, homogeneous or heterologous immunogens. In some embodiments, the homologous or heterologous immunogens are optional on the multivalent carrier, and thus the multivalent carrier comprises one or more attached linear carbohydrates but is free of any covalently attached immunogens (i.e., the number of immunogens in the complex in this embodiment is zero).

The total number of the linear carbohydrate molecules presented by a multivalent carrier can be different in different embodiments. In some embodiments, the multivalent carrier can comprise a total number of linear carbohydrate molecules about, at least, at least about, at most, or at most about 1, 2, 3, 4, 5, 6, 7, 8, 9, 10, 11, 12, 13, 14, 15, 16, 17, 18, 19, 20, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values.

In some embodiments, the number of any two different immunogen molecule s can be in a ratio from 1:100 to 100:1. In some embodiments, the ratio can be, be about, be at least, be at least about, be at most, be at most about, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, or a number or a range between any two of the values.

It should be understood that the total number of combined linear carbohydrate molecules and optional immunogen molecules presented by a nanoparticle central carrier is limited by the number of particle-forming subunits that make up the nanoparticle, such as the number of particle-forming lipids in lipid-based nanoparticles and the number of particle-forming proteins in protein-based nanoparticles. For example, encapsulin proteins from Thermotoga maritima form nanoparticles having 60-mers. Therefore, encapsulin-based nanoparticles (e.g., mi3 nanoparticle and i301 nanoparticle) can present a maximum of 60 linear carbohydrate molecules and optional immunogen molecules combined.

The immunogens of the plurality of immunogen molecules attached to a multivalent carrier can be derived from the same protein type or corresponding proteins. One skilled in the art would recognize that immunogen molecules of a same protein type may or may not have identical amino acid sequences, but generally share some sequence homology. In some embodiments, proteins of different immunogen taxonomic groups having the same function are considered the same protein type or corresponding proteins. In some embodiments, immunogens of a same protein type have at least 50% sequence identity, for example at least 65%, 70%, 80%, 90%, 95%, 98%, 99%, or more sequence identity. Alternatively, in some embodiments the plurality of immunogen molecules attached to a multivalent carrier can comprise immunogen proteins of different protein types. One skilled in the art would recognize that immunogen proteins of different protein types typically have different functions. It is understood that these embodiments include one or more linear carbohydrate molecules also presented by the carrier.

In one embodiment, the multivalent carrier complex described herein can induce the production of detectable antigen-specific antibodies selected individually and independently from the group consisting of IgG, IgA, sIgA, IgD, IgE, IgM, neutralizing antibodies, and cross-reactive neutralizing antibodies thereof.

In another embodiment, the multivalent carrier complex described herein can induce non-specific immune responses (e.g., antibody-dependent cellular cytotoxicity (ADCC), antibody-dependent cellular phagocytosis (ADCP), and antibody-mediated complement-dependent cytotoxicity (CDC) in response to an antigen. ADCC is when viral antigens on the surface of an infected cell are recognized by specific antibodies. Said antibodies signal natural killer cells to destroy the infected cell via secreted compounds (e.g., cytotoxic granules and cytokines). In ADCP, an infected cell is recognized by specific antibodies which then signal the cell to be destroyed via macrophage-directed phagocytosis. In CDC antibodies recruit and activate components of the complement cascade, leading to the formation of a Membrane Attack Complex on the cell surface and subsequent cell lysis.

The multivalent carrier complex described herein can induce broadly protective anti-pathogen responses by eliciting broadly neutralizing antibodies and/or cross-reactive neutralizing antibodies. Broadly neutralizing antibodies are antibodies that can neutralize the pathogen from a taxonomic group that is not only the same as but also differs from the taxonomic groups of the pathogen from which the immunogens used to elicit the antibodies are derived. Broadly neutralizing response can also be referred to as a heterologous neutralizing response. Cross-reactive neutralizing antibodies are antibodies which are both active and neutralizing against immunogens that were not specifically used to raise the antibody in question. That is, the antibody has neutralizing activity beyond the protein immunogens used to raise or create the antibody. In some embodiments, the multivalent carrier complexes described herein can elicit broadly neutralizing antibodies that neutralize one or more pathogens from a subfamily, genus, subgenus, species, and/or strain that differ from the subfamily, genus, subgenus, species, and/or strain of the pathogen from which the immunogens are derived to produce the multivalent carrier complexes. It is understood that these embodiments include one or more linear carbohydrate molecules also presented by the multivalent carrier.

In some embodiments, the multivalent carrier complex comprising heterologous immunogens derived from a plurality of pathogens including a first immunogen and a second immunogen that can induce heterologous binding and neutralizing responses and/or cross-reactive neutralizing antibodies against not only the first pathogen and the second pathogen, but also against one or more pathogens different from the first pathogen and the second pathogen from which the immunogens were derived (e.g., a third pathogen from which a third immunogen is derived, a fourth pathogen from which a fourth immunogen is derived, etc.). In particular, the multivalent carrier complex comprising heterologous immunogens from a plurality of pathogens not including one or more particular immunogens can induce heterologous binding and neutralizing responses and/or cross-reactive neutralizing antibodies against the one or more particular pathogens. It is understood that these embodiments include one or more linear carbohydrate molecules also presented by the multivalent carrier.

In some embodiments, the multivalent carrier complex comprising heterologous immunogens from a plurality of pathogens including a first pathogen and a second pathogen can induce about the same or comparable magnitude (e.g., about, at least, at least about 80%, 85%, 90%, 95%, 100%, 105%, 110%, 115%, 120%, 125%, 130%, 140%, 150%, or a number or a range between any two of these values, relative to one another) of immune response against the first immunogen derived from the first pathogen and/or the second immunogen derived from the second pathogen when compared to a multivalent carrier complex comprising a homologous population of a single immunogen from the first pathogen or the second immunogen from a second pathogen. In other words, co-display of immunogens from pathogens of different taxonomic groups does not diminish the immune response against a pathogen relative to multivalent carrier complexes presenting homologous antigens from the particular pathogen. In a non-limiting example, in terms of the magnitude of the immune response against a particular pathogen, it can be advantageous to conduct immunization with a complex presenting a plurality of immunogens that includes immunogens derived a pathogen as well as immunogens derived from other taxonomically distinct yet related pathogens versus immunization with a multivalent carrier complex presenting a single immunogen type. It is understood that these embodiments include one or more linear carbohydrate molecules also presented by the carrier.

In some embodiments, the multivalent carrier complex comprising heterologous immunogens from a plurality of pathogens including a first immunogen from a first pathogen and a second immunogen from a second pathogen can induce an increased magnitude of immune response against the first pathogen and/or the second pathogen when compared to a multivalent carrier comprising a homologous population of a single immunogen from the first pathogen or the second pathogen. The magnitude of immune response induced by the multivalent carrier complex can be about, at least, or at least about 0.2, 0.5, 0.9, 1.1, 1.5, 1.7, 2, 3, 4, 5, 6, 7, 8, 9, 10, fold(s), or a number or a range between any of these values, as compared to the multivalent carrier complex with a single immunogen type. In some embodiments, the magnitude of immune response induced by the multivalent carrier complex can be changed by about, at least, or at least about 5%, 10%, 20%, 30%, 50%, 75%, 100%, 110%, 120%, 150%, 200%, 250%, 300%, 400%, 500%, 600%, 700%, 800%, 900%, 1000%, or a number or a range between any of these values, as compared to that by the multivalent carrier complex with a single immunogen type. It is understood that these embodiments include one or more linear carbohydrate molecules also presented by the carrier.

In some embodiments, the multivalent carrier complex does not present an immunogen from a particular pathogen but can still produce broadly neutralizing antibodies against that particular pathogen, for example, at a comparable or even enhanced magnitude as compared to a multivalent carrier complex presenting a homologous immunogen from that particular pathogen. For example, the multivalent carrier complex comprising heterologous immunogens from a plurality of pathogens not including a first immunogen derived from the first pathogen can induce about the same or comparable magnitude (e.g., about, at least, at least about 80%, 85%, 90%, 95%, 100%, 105%, 110%, 115%, 120%, 125%, 130%, 140%, 150%, or a number or a range between any of these values, relative to one another) of immune response against the first pathogen when compared to a multivalent carrier complex comprising a homologous population of a single immunogen from the first pathogen. It is understood that these embodiments include one or more linear carbohydrate molecules also presented by the carrier.

In some embodiments, the multivalent carrier complex comprising heterologous immunogens from a plurality of pathogens not including a first immunogen derived from a first pathogen can elicit an enhanced heterologous binding and neutralizing response against the first pathogen when compared to a multivalent carrier complex comprising a homologous population of a single immunogen derived from a first pathogen and a second immunogen derived from a second pathogen. The first and second pathogens and the corresponding immunogens derived thereof are different from one another. The magnitude of the neutralizing response induced by the multivalent carrier complex comprising heterologous immunogens can be about, at least, or at least about 0.2, 0.5, 0.9, 1.1, 1.5, 1.7, 2, 3, 4, 5, 6, 7, 8, 9, 10-fold, or a number or a range between any of these values, as compared to the response by the multivalent carrier complex with a single immunogen type. In some embodiments, the magnitude of the immune response induced by the multivalent carrier comprising heterologous immunogens can be increased by about, at least, or at least about 5%, 10%, 20%, 30%, 50%, 75%, 100%, 110%, 120%, 150%, 200%, 250%, 300%, 400%, 500%, 600%, 700%, 800%, 900%, 1000%, or a number or a range between any of these values, as compared to the response by the multivalent carrier complex with a single immunogen type. It is understood that these embodiments include one or more linear carbohydrate molecules also presented by the carrier.

In some embodiments, the multivalent carrier complex comprising heterologous immunogens from a plurality of pathogens including a first immunogen derived from a first pathogen and a second immunogen derived from a second pathogen can elicit a substantially enhanced neutralizing response against the first pathogen and/or the second pathogen when compared to an unattached immunogen from the first pathogen or the second pathogen. The magnitude of the neutralizing response induced by the multivalent carrier complex can be about, at least, or at least about 1, 2, 3, 4, 5, 6, 7, 8, 9, 10, 50, 100, 200, 300, 400, 500, 600, 700, 800, 900, 1,000-fold, or a number or a range between any of these values, greater than the response by the unattached immunogen. It is understood that these embodiments include one or more linear carbohydrate molecules also presented by the carrier.

In one embodiment, the plurality of coronavirus immunogens attached to a multivalent carrier can be of a same protein type or corresponding proteins. Coronavirus immunogens of a same protein type may or may not have identical amino acid sequences, but generally share some sequence homology. For example, the coronavirus S proteins of different coronaviruses are of a same protein type or corresponding proteins. As another example, envelope proteins from different coronaviruses are considered the same protein type or corresponding proteins. In some embodiments, proteins of different coronavirus taxonomic groups having the same function are considered the same protein type or corresponding proteins. Another example is the receptor binding domains of the different coronaviruses. In some embodiments, coronavirus immunogens of a same protein type have at least 50% sequence identity, for example at least 65%, 70%, 80%, 90%, 95%, 98%, 99%, or more sequence identity.

In some embodiments, the immunogen used herein can be derived from a coronavirus spike (S) protein or a portion thereof. A S protein is one of four major structural proteins covering the surface of each virion. The S protein, comprising a S1 subunit and a S2 subunit, is a highly glycosylated, type I transmembrane protein capable of binding to a host-cell receptor and mediates viral entry. The S protein comprises a domain referred to as the RBD that mediates the interaction with the host-cell receptor to enter the host cell after one or more RBDs adopts an “up” position to bind the host receptor. It is believed that after binding the receptor, a nearby host protease cleaves the spike, which releases the spike fusion peptide, facilitating virus entry. Known host receptors for coronaviruses (e.g., Beta-coronaviruses) include angiotensin-converting enzyme 2 (ACE2), dipeptidyl peptidase-4 (DPP4) or sialic acids. In some embodiments the immunogen is derived from a coronavirus S protein or a portion thereof, comprising or consisting of an amino acid sequence having, having about, having at least, or having at least about, 80%, 85%, 90%, 95%, 98%, 99%, or more sequence identity to the amino acid sequence of any of the coronavirus S proteins from one or more coronaviruses selected from SARS-CoV, SARS-CoV2, WIV1, LYRa11, Rs7327, Rs4231, Rs4084, SHC014, As6526, Yunnan 2011, Shaanxi 2011, 279-2005, Rs4237, Rs4081, Rp3, Rs4247, HKU3-8, HKU3-13, GX2013, Longquan-140, YN2013, Rf4092, ZXC21, ZC45, JL2012, HuB2013, Rf1, HeB2013, 273-2005, and BM48-31.

In some embodiments, the immunogen used herein can be derived from a coronavirus spike RBD or a portion or a variant thereof. The coronavirus spike RBD or a portion thereof used herein can be of any species or strains in the genus of alphacoronavirus and/or betacoronavirus. For example, the coronavirus spike RBD protein or a portion thereof can be of any species or strains in the subgenus of Embecovirus, including but not limited to, Betacoronavirus 1 (e.g., Bovine coronavirus and human coronavirus OC43), China Rattus coronavirus HKU24, Human coronavirus HKU1, Murine coronavirus (e.g., mouse hepatitis virus), and Myodes coronavirus 2JL14. The coronavirus spike RBD protein or a portion thereof can be of any viral species or strains in the subgenus of Sarbecovirus, including but not limited to, SARS-CoV, SARS-CoV2, 16BO133, Bat SARS CoV Rf1, Bat coronavirus HKU3 (BtCoV HKU3), LYRa11, Bat SARS-CoV/Rp3, Bat SL-CoV YNLF_31C, Bat SL-CoV YNLF_34C, SHCO14-CoV, WIV1, WIV16, Civet SARS-CoV, Rc-o319, SL-ZXC21, SL-ZC45, Pangolin SARSr-COV-GX, Pangolin SARSr-COV-GD, RshSTT182, RshSTT200, RacCS203, RmYNO2, RpYN06, RaTG13, Bat CoV BtKY72, and Bat CoV BM48-31. The coronavirus spike RBD protein or a portion thereof can be of any viral species or strains in the subgenus of Merbecovirus, including but not limited to, Hedgehog coronavirus 1, MERS-CoV, Pipistrellus bat coronavirus HKU5, and Tylonycteris bat coronavirus HKU4. The coronavirus spike RBD protein or a portion thereof can be of any viral species or strains in the subgenus of Nobecovirus, including but not limited to, Eidolon bat coronavirus C704, Rousettus bat coronavirus GCCDC1, and Rousettus bat coronavirus HKU9. The coronavirus spike RBD protein or a portion thereof can be of any viral species or strains in the subgenus of Hibecovirus, including but not limited to, Bat Hp-betacoronavirus Zhejiang 2013.

In an embodiment, the immunogen used herein can be derived from a coronavirus spike RBD protein or a portion thereof from any viral species or strain in any one of the phylogenetically clustered clades of lineage B coronavirus (Sarbecovirus). For example, the coronavirus spike RBD protein or a portion thereof can be of any species or strain in clade 1, including but not limited to SARS-CoV, WIV1, LYRa11, Rs7327, Rs4231, Rs4084, and SHC014. The coronavirus spike RBD protein or a portion thereof can be of any species or strain in clade 2, including but not limited to, As6526, Yunnan 2011, Shaanxi 2011, 9-2005, Rs4237, Rs4081, Rp3, Rs4247, HKU3-8, HKU3-13, GX2013, Longquan-140, YN2013, Rf4092, ZXC21, ZC45, JL2012, HuB2013, Rf1, HeB2013, and 273-2005. The coronavirus spike RBD protein or a portion thereof can be of any species or strain in clade 1/2, including but not limited to SARS-CoV2. The coronavirus spike RBD protein or a portion thereof can be of any species or strain in clade 3, including but not limited to BM48-31.

In some embodiments, the immunogen used herein can be derived from a coronavirus spike RBD protein or a portion thereof can be from a coronavirus selected from SARS-CoV, SARS-CoV2, WIV1, LYRa11, Rs7327, Rs4231, Rs4084, SHC014, As6526, Yunnan 2011, Shaanxi 2011, 279-2005, Rs4237, Rs4081, Rp3, Rs4247, HKU3-8, HKU3-13, GX2013, Longquan-140, YN2013, Rf4092, ZXC21, ZC45, JL2012, HuB2013, Rf1, HeB2013, 273-2005, and BM48-31.

In some embodiments, the immunogen used herein can, for example, can be derived from a coronavirus nucleocapsid protein (N protein) or a portion thereof. The N protein is a multifunctional RNA-binding protein required for viral RNA transcription, replication, and packaging. The N protein consists of three domains, an N-terminal RNA-binding domain, a central intrinsically disordered region, followed by a C-terminal dimerization domain. The RNA-binding domain contains multiple positively charged binding surfaces that form charged interactions with RNA promoting its helical arrangement. In some embodiments, the immunogen is derived from a coronavirus N protein or a portion thereof, comprising or consisting of an amino acid sequence having, having about, having at least, or having at least about, 80%, 85%, 90%, 95%, 98%, 99% or more, sequence identity to the amino acid sequence of any of the coronavirus N proteins from one or more coronaviruses selected from SARS-CoV, SARS-CoV2, WIV1, LYRa11, Rs7327, Rs4231, Rs4084, SHC014, As6526, Yunnan 2011, Shaanxi 2011, 279-2005, Rs4237, Rs4081, Rp3, Rs4247, HKU3-8, HKU3-13, GX2013, Longquan-140, YN2013, Rf4092, ZXC21, ZC45, JL2012, HuB2013, Rf1, HeB2013, 273-2005, and BM48-31.

In some embodiments, the immunogen used herein can be derived from a coronavirus membrane protein (M protein) or a portion thereof. The M protein is the most abundant structural protein and defines the shape of the viral envelope. The M protein is regarded as the central organizer of the viral assembly, interacting with other major coronaviral structural proteins. In some embodiments, the immunogen is derived from a coronavirus M protein or a portion thereof comprising or consisting of an amino acid sequence having, having about, having at least, having at least about, 80%, 85%, 90%, 95%, 98%, 99% or more, sequence identity to the amino acid sequence of any of the coronavirus M proteins from one or more coronaviruses selected from SARS-CoV, SARS-CoV2, WIV1, LYRa11, Rs7327, Rs4231, Rs4084, SHC014, As6526, Yunnan 2011, Shaanxi 2011, 279-2005, Rs4237, Rs4081, Rp3, Rs4247, HKU3-8, HKU3-13, GX2013, Longquan-140, YN2013, Rf4092, ZXC21, ZC45, JL2012, HuB2013, Rf1, HeB2013, 273-2005, and BM48-31.

In some embodiments, the immunogen used herein can be derived from a coronavirus envelope protein (E protein) or a portion thereof. The E protein is a small membrane protein and minor component of virus particles. Without being bound to any theory, it is believed that the E protein plays roles in virion assembly and morphogenesis, alteration of the membrane of host cells and virus-host cell interaction.

In some embodiments, the immunogen used herein can be derived from a coronavirus hemagglutinin-esterase protein (HE protein) or a portion thereof. The HE protein, which is an envelope protein, mediates reversible attachment to O-acetylated sialic acids by acting both as lectins and receptor-destroying enzymes. In some embodiments, the immunogen used herein can be derived from a coronavirus HE protein or a portion thereof comprising or consisting of an amino acid sequence having, having about, having at least, having at least about, 80%, 85%, 90%, 95%, 98%, 99% or more, sequence identity to the amino acid sequence of any of the coronavirus HE proteins from one or more coronaviruses selected from SARS-CoV, SARS-CoV2, WIV1, LYRa11, Rs7327, Rs4231, Rs4084, SHC014, As6526, Yunnan 2011, Shaanxi 2011, 279-2005, Rs4237, Rs4081, Rp3, Rs4247, HKU3-8, HKU3-13, GX2013, Longquan-140, YN2013, Rf4092, ZXC21, ZC45, JL2012, HuB2013, Rf1, HeB2013, 273-2005, and BM48-31.

In some embodiments, the immunogen used herein can be derived from a coronavirus papain-like protease or a portion thereof. The coronavirus papain-like protease is one of several nonstructural proteins and is responsible for processing of viral proteins into functional, mature subunits during maturation. For example, the coronavirus papain-like protease can cleave a site at the amino-terminal end of the viral replicase region. In addition to its role in viral protein maturation, papain-like protease exhibits both a deubiquitinating and deISG15ylating activity. In vivo, this protease antagonizes innate immunity by acting on IFN beta and NF-kappa B signaling pathways. In some embodiments, the immunogen used herein can be derived from a coronavirus papain-like protease or a portion thereof comprising or consisting of an amino acid sequence having, having about, having at least, having at least about, 80%, 85%, 90%, 95%, 98%, 99% or more, sequence identity to the amino acid sequence of any of the coronavirus papain-like proteases from one or more coronaviruses selected from SARS-CoV, SARS-CoV2, WIV1, LYRa11, Rs7327, Rs4231, Rs4084, SHC014, As6526, Yunnan 2011, Shaanxi 2011, 279-2005, Rs4237, Rs4081, Rp3, Rs4247, HKU3-8, HKU3-13, GX2013, Longquan-140, YN2013, Rf4092, ZXC21, ZC45, JL2012, HuB2013, Rf1, HeB2013, 273-2005, and BM48-31.

In some embodiments, the immunogen used herein can be derived from a coronavirus 3CL protease or a portion thereof. The 3CL protease is another main protease in addition to the papain-like protease and is required for processing of viral polypeptides into distinct, functional proteins. In some embodiments, the 3CL protease is a SARS-CoV-2 3CL Protease, which is a C30-type cysteine protease located within the non-structural proteins 3 (NS3) region of the viral polypeptide. Analysis of the Coronavirus genome reveals at least 11 sites of cleavage for the 3CL protease, many containing the amino acid sequence LQ[S/A/G]. In some embodiments, the immunogen used herein can be derived from a coronavirus 3CL protease or a portion thereof comprising or consisting of an amino acid sequence having, having about, having at least, having at least about, 80%, 85%, 90%, 95%, 98%, 99% or more, sequence identity to the amino acid sequence of any of the coronavirus 3CL proteases from one or more coronaviruses selected from SARS-CoV, SARS-CoV2, WIV1, LYRa11, Rs7327, Rs4231, Rs4084, SHC014, As6526, Yunnan 2011, Shaanxi 2011, 279-2005, Rs4237, Rs4081, Rp3, Rs4247, HKU3-8, HKU3-13, GX2013, Longquan-140, YN2013, Rf4092, ZXC21, ZC45, JL2012, HuB2013, Rf1, HeB2013, 273-2005, and BM48-31.

In some embodiments, the plurality of coronavirus derived immunogens covalently attached to a multivalent carrier can comprise coronavirus derived proteins of different protein types. For example, the plurality of coronavirus derived immunogens covalently attached to a multivalent carrier can be derived from coronavirus S proteins or portions thereof as well as other coronavirus proteins such as a coronavirus N protein or a portion thereof, a coronavirus HE protein or a portion thereof, a coronavirus papain-like protease or a portion thereof, a coronavirus 3CL protease or a portion thereof, a coronavirus M protein or a portion thereof, or a combination thereof.

One or more of the plurality of coronavirus derived immunogens, or each of the plurality of coronavirus derived immunogens, can have a sequence identity of about, at least, or at least about 65%, 70%, 75%, 80%, 85%, 90%, 95%, 98%, or 99% with one another. In some embodiments, the plurality of coronavirus derived immunogens each comprise a coronavirus S protein RBD or a portion thereof, the coronavirus S protein RBDs or portions thereof having a sequence identity of about, at least, or at least about, 65%, 70%, 75%, 80%, 85%, 90%, 95%, 98%, or 99% with one another.

The embodiments disclosed herein focus on the coronavirus-derived immunogen portion presented by a nanoparticle carrier, but it is understood that these embodiments include one or more linear carbohydrate molecules that is also presented by the carrier.

In another embodiment, the plurality of Borrelia derived immunogens covalently attached to a multivalent carrier can be of a same protein type or corresponding proteins. Borrelia derived immunogens of a same protein type may or may not have identical amino acid sequences, but generally share some sequence homology. For example, the Borrelia outer surface protein C (OspC) of different Borrelia strains are of a same protein type or corresponding proteins. As another example, the Borrelia outer surface protein A (OspA) proteins from different Borrelia strains are considered the same protein type or corresponding proteins. In some embodiments, proteins of different Borrelia taxonomic groups having the same function are considered the same protein type or corresponding proteins. In some embodiments, Borrelia immunogens of a same protein type have at least 50% sequence identity, for example at least 65%, 70%, 80%, 90%, 95%, 98%, 99%, or more sequence identity.

In one embodiment, the plurality of OspC types are associated with human Borrelia infection and are selected from the group consisting of: T, U, B, E, K, H, N, C, and M; is further aspects, the plurality of OspC types are associated with human Borrelia infection and are selected from the group consisting of: Pwa, Pli, PBes, Pki, PFim, Smar, HT22, A and K; and in yet further aspects, the plurality of OspC types are associated with canine Borrelia infection and are selected from the group consisting of types I, H, N, C, M, D, and F.

In another embodiment, the plurality of Borrelia derived immunogens attached to a multivalent carrier can comprise Borrelia derived proteins of different protein types. For example, the plurality of Borrelia derived immunogens attached to a multivalent carrier can comprise OspC or portions thereof, as well as other Borrelia proteins such as OspA, OspB, OspE, or portions thereof.

One or more of the plurality of Borrelia derived immunogens, or each of the plurality of Borrelia derived immunogens, can have a sequence identity of about, at least, or at least about 50%, 65%, 70%, 75%, 80%, 85%, 90%, 95%, 98%, or 99% with one another. In some embodiments, the plurality of Borrelia derived immunogens each comprise OspA or a portion thereof, OspC or a portion thereof, OspE or a portion thereof, and OspB or a portion thereof having a sequence identity of about, at least, or at least about, 50%, 65%, 70%, 75%, 80%, 85%, 90%, 95%, 98%, or 99% with one another.

One or more of the plurality of Babesia derived immunogens, or each of the plurality of Babesia derived immunogens, can have a sequence identity of about, at least, or at least about 50%, 65%, 70%, 75%, 80%, 85%, 90%, 95%, 98%, or 99% with one another. In some embodiments, the plurality of Babesia derived immunogens are derived from, but are not limited to, BMSA, BmSA1, BmSP44, BmPROF, BboPROF, BbigPROF, BmAMA-1, BmRON2, Bm2D41, BmSERA1, BmMCFPR1, BmPiβS1, BmBAHCS1, BboPROF, BdAMA1, BdP0, N-terminal and C-terminal fragments of BmRON2, Babesia microti methionine aminopeptidase protein 1, or a portion thereof having a sequence identity of about, at least, or at least about, 50%, 65%, 70%, 75%, 80%, 85%, 90%, 95%, 98%, or 99% with one another.

One or more of the plurality of Chlamydia derived immunogens, or each of the plurality of Chlamydia derived immunogens, can have a sequence identity of about, at least, or at least about 50%, 65%, 70%, 75%, 80%, 85%, 90%, 95%, 98%, or 99% with one another. In some embodiments, the plurality of Chlamydia derived immunogens are derived from, but are not limited to, OmpA, CPAF, PmpG, Chlamydia trachomatis OmpA serovars D, E, F, G, Chlamydia trachomatis CPAF, Chlamydia trachomatis PmpG, or a portion thereof having a sequence identity of about, at least, or at least about, 50%, 65%, 70%, 75%, 80%, 85%, 90%, 95%, 98%, or 99% with one another.

One or more of the plurality of Orthomyxoviridae derived immunogens, or each of the plurality of Picornaviridae derived immunogens, can have a sequence identity of about, at least, or at least about 50%, 65%, 70%, 75%, 80%, 85%, 90%, 95%, 98%, or 99% with one another. In some embodiments, the plurality of Orthomyxoviridae immunogens is derived from, but not limited to, VP1, VP2, VP3, and VP4. having a sequence identity of about, at least, or at least about, 50%, 65%, 70%, 75%, 80%, 85%, 90%, 95%, 98%, or 99% with one another.

One or more of the plurality of human immunodeficiency virus derived immunogens, or each of the plurality of human immunodeficiency virus derived immunogens, can have a sequence identity of about, at least, or at least about 50%, 65%, 70%, 75%, 80%, 85%, 90%, 95%, 98%, or 99% with one another. In some embodiments, the plurality of human immunodeficiency virus immunogens is derived from, but not limited to, ENV, GP160, Gag, Pol, Gag-Pol-Nef, having a sequence identity of about, at least, or at least about, 50%, 65%, 70%, 75%, 80%, 85%, 90%, 95%, 98%, or 99% with one another.

One or more of the plurality of Human orthopneumovirus derived immunogens, or each of the plurality of human immunodeficiency virus derived immunogens, can have a sequence identity of about, at least, or at least about 50%, 65%, 70%, 75%, 80%, 85%, 90%, 95%, 98%, or 99% with one another. In some embodiments, the plurality of Human orthopneumovirus immunogens is derived from, but not limited to RSVPreF3, having a sequence identity of about, at least, or at least about, 50%, 65%, 70%, 75%, 80%, 85%, 90%, 95%, 98%, or 99% with one another.

In order to facilitate the understanding of the present invention, the following additional definitions are provided: A peptide, as defined by IUPAC, is amides derived from two or more amino carboxylic acid molecules (the same or different) by formation of a covalent bond from the carbonyl carbon of one to the nitrogen atom of another with formal loss of water. The term is usually applied to structures formed from α-amino acids, but it includes those derived from any amino carboxylic acid. One skilled in the art will recognize that said definition is independent of the stereochemistry within the polypeptide chain, and as such would be equally applicable to peptides and proteins derived from L-amino acids and D-amino acids. Polypeptides, as defined by IUPAC, are naturally occurring and synthetic peptides containing ten or more amino acid residues. Proteins, as defined by IUPAC, are naturally occurring and synthetic polypeptides having molecular weights greater than about 10000 Da (the limit is not precise) (IUPAC, 1997).

As used herein, a serotype is defined as a variation within a microorganism species, distinguished by the humoral immune response. The serotype classification of bacteria or viruses is based on their surface antigens and was established before the availability of other techniques, such as genome sequencing or mass spectrometry. Antibodies generated to one serotype do not usually efficiently protect against another serotype. Serotypes have been described in many viral species and generally correspond to genotypes (Simon-Loriere et al, 2022).

As used herein, “sequence identity” or “identity” in the context of two nucleic acid or polypeptide sequences makes reference to the nucleotide bases or residues in the two sequences that are the same when aligned for maximum correspondence over a specified comparison window. Methods of alignment of sequences for comparison are well known in the art. Various programs and alignment algorithms are described in: Smith et al., 1981; Needleman et al., 1970; Pearson et al., 1988; Higgins et al., 1988; Higgins et al., 1989; Corpet, 1988; Huang et al., 1992; Pearson, 1994; and Altschul, 1990. When percentage of sequence identity or similarity is used in reference to proteins, it is recognized that residue positions which are not identical often differ by conservative amino acid substitutions, where amino acid residues are substituted with a functionally equivalent residue of the amino acid residues with similar physiochemical properties and therefore do not change the functional properties of the molecule. A functionally equivalent residue of an amino acid used herein typically can refer to other amino acid residues having physiochemical and stereochemical characteristics substantially similar to the original amino acid. The physiochemical properties include water solubility (hydrophobicity or hydrophilicity), dielectric and electrochemical properties, physiological pH, partial charge of side chains (positive, negative, or neutral), and other properties identifiable to one of skill in the art. The stereochemical characteristics include spatial and conformational arrangement of the amino acids and their chirality. For example, glutamic acid is considered to be a functionally equivalent residue to aspartic acid in the sense of the current disclosure. Tyrosine and tryptophan are considered as functionally equivalent residues to phenylalanine. Arginine and lysine are considered as functionally equivalent residues to histidine.

The term “substantially identical” as used herein in the context of two or more sequences refers to a specified percentage of amino acid residues or nucleotides that are identical or functionally equivalent, such as about, at least or at least about 65% identity, optionally, about, at least or at least about 70%, 75%, 80%, 85%, 90%, 95%, or 99% identity over a specified region or over the entire sequence.

As used herein, the term “variant” refers to a polynucleotide or polypeptide having a sequence substantially similar or identical to a reference (e.g., the parent) polynucleotide or polypeptide. In the case of a polynucleotide, a variant can have deletions, substitutions, additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR), high throughput sequencing, Sanger sequencing, and hybridization techniques. Variant polynucleotides also include synthetically derived polynucleotides, such as those generated, for example, by using site-directed mutagenesis. Generally, a variant of a polynucleotide, including, but not limited to, a DNA, can have at least, or at least about, 50%, 55%, 60%, 65%, 70%, 75%, 80%, 85%, 90%, 91%, 92%, 93%, 94%, 95%, 96%, 97%, 98%, 99%, or more sequence identity to the reference polynucleotide as determined by sequence alignment programs known in the art. In the case of a polypeptide, a variant can have deletions, substitutions, additions of one or more amino acids in comparison to the reference polypeptide. Similarities and/or differences in sequences between a variant and the reference polypeptide can be detected using conventional techniques known in the art, for example Western blot, Edman degradation, and mass spectroscopy. A variant of a polypeptide can have, for example, at least, or at least about, 60%, 65%, 70%, 75%, 80%, 85%, 90%, 91%, 92%, 93%, 94%, 95%, 96%, 97%, 98%, 99%, or more sequence identity to the reference polypeptide as determined by sequence alignment programs known in the art.

The multivalent carriers herein described can be prepared using standard molecular biology procedures known to the person skilled in the art, as well as the protocols exemplified herein. In some embodiments, particle-forming subunits and/or the immunogens or linear carbohydrates can be produced by liquid-phase or solid-phase chemical protein synthetic methods known to those of skill in the art.

Production of the particle-forming subunits and/or the immunogens can use recombinant DNA technology well known in the art. For example, a tagged immunogen or immunogen functionalized with a protein tag can be synthesized using biosynthetic methods, such as cell-based or cell-free methods known to the person skilled in the art. A tagged immunogen can be produced using an expression vector comprising a nucleic acid molecule encoding the immunogen. The nucleic acid sequence encoding the particle-forming subunits and/or the immunogens can be operably linked to appropriate regulatory elements including, but not limited to, a promoter, enhancer, transcription initiation site, termination site, and translation initiation site. The vector can also comprise a nucleic acid molecule encoding one or more protein tags (e.g., a poly(His) tag, SpyTag). In some embodiments, the vector can additionally include a nucleic acid sequence encoding a trimerization motif (e.g., a foldon trimerization domain from T4 fibritin or viral capsid protein SHP). The vector can also comprise a nucleic acid sequence encoding a signal peptide that directs the protein into the proper cellular pathway, such as a signal peptide for secretion of the expressed protein into supernatant medium. The vector may comprise one or more selectable marker genes such as a gene providing ampicillin resistance or kanamycin resistance. Methods for the construction of nucleic acid constructs are well known. See, for example, Molecular Cloning: a Laboratory Manual, 3^{rd} edition and Current Protocols in Molecular Biology, 1994-1998. Protein biosynthesis of tagged immunogens can be performed by providing cell-based or cell-free protein translation systems with the expression vectors encoding the tagged immunogens. Similarly, a tagged particle-forming protein can be produced using an expression vector comprising a nucleic acid sequence encoding a particle-forming subunit and a nucleic acid sequence encoding a protein tag (e.g., SpyCatcher). In an exemplary embodiment, the multivalent carriers are produced following the protocols described in Cohen et al, 2021

In some embodiments, constructs expressing the carrier subunit and the immunogens can be introduced together into a host or transformation-competent cell. Multivalent carriers can be generated as a result of conjugation of the expressed immunogens to the self-assembled nanoparticles through a functional group pair or a reactive moiety pair described herein (e.g., SpyTag/SpyCatcher). It is understood in these embodiments that one or more linear carbohydrate molecules are also covalently attached to the carrier.

Nanoparticle carriers (e.g., nanoparticles with SpyCatcher), linear carbohydrate molecules (e.g., a SpyTagged linear carbohydrate molecule), and immunogens (e.g., SpyTagged protein immunogen) can, for example, be prepared separately and then incubated under a condition (e.g., in a TBS buffer at room temperature) for a certain time period (e.g., about, at least, or at least about 30 seconds, 1 minutes, 2 minutes, 3 minutes., 5 minutes, 10 minutes, 15 minutes, 30 minutes, 45 minutes, 1 hour, 2 hours, 5 hours, 10 hours, 12 hours, 15 hours, 24 hours, 48 hours, 72 hours, 96 hours) to allow for the conjugation of the carriers and the linear carbohydrate molecules and/or the immunogen molecules. In some embodiments, the nanoparticle carrier, linear carbohydrate molecules, and immunogens are incubated concurrently. In other embodiments, the nanoparticle and linear carbohydrate molecules are incubated together, and the immunogens are incubated with the pre-formed nanoparticle carbohydrate complex at some later time. In some embodiments, the linear carbohydrate molecules and the immunogen molecules are provided in an excess amount as compared to the particle-forming subunits of the carriers, such as 1-fold, 2-fold, 3-fold, 4-fold, 5-fold or greater than the particle-forming subunits. The ratio of the linear carbohydrate molecules to the particle-forming subunits of the carriers and the ratio of the immunogen molecules to the particle-forming subunits of the carriers may be the same or different.

A vaccine composition is a pharmaceutical composition that can elicit a prophylactic (e.g., to prevent or delay the onset of a disease, or to prevent the manifestation of clinical or subclinical symptoms thereof) or therapeutic (e.g., suppression or alleviation of symptoms) immune response in a subject. Provided herein is a vaccine composition comprising a multivalent carrier as herein described. The vaccine composition may contain one or more compatible and pharmaceutically acceptable carriers. The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition, or vehicle, such as a liquid or solid filler, diluent, excipient, solvent, or encapsulating material, involved in carrying or transporting the subject chemical from one organ, or portion of the body, to another organ, or portion of the body. Each pharmaceutically acceptable carrier must be “acceptable” in the sense of being compatible with the other ingredients of the formulation and not injurious to the subject. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth: (5) malt; (6) gelatin; (7) talc; (8) excipients, such as cocoa butter and suppository waxes; (9) oils, such as peanut oil, cottonseed oil, safflower oil, sesame oil, olive oil, corn oil, sunflower oil, and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; (21) linear carbohydrates such as dextrans, chitin, partially deacylated chitin, chitosan, partially acylated chitosan, hyaluronic acid, keratin, keratin sulfate, chondroitin, chondroitin sulfate, dermatan, dermatan sulfate, and heparin, and derivatives thereof; and (22) other non-toxic compatible substances employed in vaccine formulations. In some embodiments, the pharmaceutically acceptable carrier is a carbohydrate (i.e., hyaluronic acid, partially deacylated chitin, chitosan, partially acylated chitosan, and derivatives thereof) which may vary (i.e., molecular weight, functionalization) from the linear carbohydrate adjuvant. One skilled in the art would appreciate that one or more pharmaceutically acceptable carriers may be used in a vaccine composition.

In some embodiments, a pharmaceutically acceptable carrier comprises a pharmaceutical acceptable salt. As used herein, a “pharmaceutical acceptable salt” includes a salt of an acid from of one of the components of the compositions herein described. These include organic or inorganic acid salts of the amines. Preferred acid salts are hydrochlorides, acetates, salicylates, nitrates, tartrates, malonates, hydrobromides, sulfates, carbonates, and phosphates. Other suitable pharmaceutically acceptable salts are well known to those skilled in the art and include basic salts of a variety of inorganic and organic acids.

The vaccine composition can further comprise appropriate adjuvants. Adjuvant refers to any immunomodulating substance capable of being combined with the multivalent carriers complex herein described to enhance, improve or otherwise modulate an immune response in a subject. In another embodiment, a linear carbohydrate is an adjuvant, and also may be a pharmaceutically acceptable carrier, and/or a vehicle. That is, non-attached linear carbohydrate is part of the vaccine composition.

The vaccine composition can be formulated for a variety of modes of administration. Techniques for formulation and administration can be found, for example, in “Remington's Pharmaceutical Sciences.” In an embodiment, the vaccine compositions thereof can be administered to a subject systematically. The wording “systemic administration” as used herein indicates any route of administration by which a vaccine composition is brought in contact with the body of the individual, so that the resulting composition location in the body is systemic (i.e., not limited to a specific tissue, organ, or other body part where the vaccine is administered). Systemic administration can be continuous, chronic, short, or intermittent. In some embodiments, the vaccine compositions of the present disclosure may be specially formulated for administration in solid or liquid form, including those adapted for the following:
(1) Enteral administration, a route of administration wherein the vaccine composition is delivered via the digestive tract, and includes, but is not limited to, oral administration, administration by gastric feeding tube, administration by duodenal feeding tube, gastrostomy, enteral nutrition, and rectal administration. Enteral administration includes administration through the mouth, through a gastrostomy tube or jejunostomy tube if through a pre-programmed pump or through a syringe, or a metered dose inhaler. Enteral administration also includes, but is not limited to, drenches (aqueous or non-aqueous solutions or suspensions), tablets, boluses, powders, granules, pastes, pessaries, creams, foams, tablets, gels, or suppositories;
(2) Parenteral administration, a route of administration wherein the vaccine composition is delivered via injection or infusion, and includes, but is not limited to, subcutaneous administration, intravenous administration, intramuscular administration, intraarterial administration, intraperitoneal administration, or intracranial administration, i.e., intrathecal or intracerebroventricular administration. Parenteral administration includes, but not limited to, drenches (aqueous or non-aqueous solutions or suspensions), and boluses;
(3) Topical administration, a route of administration wherein the vaccine composition is delivered via, but not limited to, direct application to the external epidermis, dermis, mucous membrane, via buccal contact, via sublingual contact, or via ocular contact, but excludes intrapulmonary administration and intranasal administration. Topical administration includes, but is not limited to, application of creams, ointments, spray gels, lotions, via patches, via microneedles, or other formulations; (4) Intravaginal administration, a route of administration wherein the vaccine composition is delivered inside the vagina. Intravaginal administration includes, but is not limited to, pessaries, creams, foams, tablets, gels, or suppositories; or,
(5) Intranasal administration, a route of administration wherein the vaccine composition is delivered via intranasal and intrapulmonary administration. Intranasal administration to the body refers to the means of direct administration through the nostrils or the mouth resulting in contact of the vaccine composition with the nasal mucosa or other aspects of the nasal cavity. Intranasal administration includes, but is not limited to, an aqueous aerosol, liposomal preparation, inhalant, liquid drops, or other formulations that provide for contact of the vaccine composition with the mucosa.

Formulations useful in the methods of the present disclosure includes those suitable for enteral, parenteral, topical, intravaginal, and intranasal administration. The formulations may conveniently be presented in unit dosage form and may be prepared by any methods well known in the art of pharmacy. The amount of active ingredient which can be combined with a pharmaceutically acceptable carrier to produce a single dosage form will vary depending upon the host being treated, and the particular mode of administration. The amount of active ingredient, which can be combined with a pharmaceutically acceptable carrier to produce a single dosage form will generally be that amount of the immunogen which produces a therapeutic effect or an immune response. Generally, out of one hundred percent, this amount will range from about 1% to about 99% of active ingredient, preferably from about 5% to about 70%, most preferably from about 5% to about 25%.

Formulations suitable for vaccine composition administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), and/or as mouth washes and the like.

In solid dosage forms for vaccine composition administration (capsules, tablets, pills, dragees, powders, granules and the like), the nanoparticle carrier complex is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the vaccine compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for vaccine composition administration include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols, and fatty acid esters of sorbitan, and mixtures thereof.

The vaccine composition can be presented in a unit dosage form, e.g., in ampoules or in multi-dose containers, with an optionally added preservative. The vaccine composition can further be formulated as suspensions, solutions, or emulsions in oily or aqueous vehicles, and may contain other agents including suspending, stabilizing and/or dispersing agents.

The vaccine composition can be formulated as an inhalant, liquid drops, aerosols, or other formulations. When administered as a liquid, compositions of the invention may be administered as an aqueous solution, e.g., a saline solution. The parameters of the formulation (e.g., pH, osmolarity, viscosity, etc.) may be adjusted as necessary to facilitate the delivery of the compositions of the invention.

The vaccine compositions disclosed herein can be employed in a variety of therapeutic or prophylactic applications to stimulate an immune response in a subject in need, to treat or prevent a pathogen infection in a subject in need, and/or to treat or prevent a disease or disorder caused by a pathogen in a subject in need.

As used herein, the term “treatment” or “treat” refers to an intervention made in response to a disease, disorder or physiological condition manifested by a patient. The aim of treatment may include, but is not limited to, one or more of the alleviation or prevention of symptoms, slowing or stopping the progression or worsening of a disease, disorder, or condition and the remission of the disease, disorder, or condition. The term “treat” and “treatment” includes, for example, therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. As used herein, the term “prevention” refers to any activity that reduces the burden of the individual later expressing those symptoms. This can take place at primary, secondary and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications. The term “prevent” does not require the 100% elimination of the possibility of an occurrence or an event, including disease transmission and retransmission. Rather, it denotes that the likelihood of the occurrence of the event has been reduced in the presence of the compound or method. The term “condition” as used herein indicates a physical status of the body of an individual (as a whole or as one or more of its parts), that does not conform to a standard physical status associated with a state of complete physical, mental, and social well-being for the individual. Conditions herein described include, but are not limited to, disorders and diseases wherein the term “disorder” indicates a condition of the living individual that is associated to a functional abnormality of the body or of any of its parts, and the term “disease” indicates a condition of the living individual that impairs normal functioning of the body or of any of its parts and is typically manifested by distinguishing signs and symptoms.

The terms “subject”, “subject in need”, and “individual” as used herein refer to an animal. The term “animal” is intended to include mammals, birds, fish, amphibians, reptiles, and the like. Animal or host includes humans and non-human mammals. Non-human mammals include, but are not limited to, Equidae (e.g., horse, zebra, asses), Canidae (e.g., dogs, wolves, foxes, coyotes, jackals), Felidae (e.g., domestic cats, wild cats including cheetahs, lions, tigers, leopard, and lynx), Bovidae (e.g., sheep, cattle, goats, buffalo, bison, wild oxen), Suidae (e.g., pig), Leporidae (e.g., rabbit), Primates (e.g., prosimian, tarsier, monkey, gibbon, ape, gorilla, chimpanzee, macaques, lemur), Rodentia (e.g. mouse, rat, hamster, marmot, squirrel, beavers, gerbils), Mustelae (e.g. weasel, ferret, mink), Cingulatae (e.g., armadillo), Chiropterae (e.g., bats), and Cervidae (e.g. deer, elk, moose). Other animals include, but are not limited to, Osteichthyes, Chondrichthyes, Aves (e.g., chicken, duck, goose, turkey, quail, pheasant, parrot, finches, hawk, crow, ostrich, emu, and cassowary), Amphibia (e.g., frogs, toads, newts, salamanders), and Reptilia (e.g., turtles, tuatara, lizards, snakes). The term “animal” also includes an individual animal in all stages of development, including embryonic and fetal stages.

In some embodiments, the subject or individual has been exposed to a pathogen. The term “exposed” indicates the subject has come in contact with a person or an animal that is infected with a pathogen. Exposed may also mean that the subject or individual has come in contact with an environmental pathogen, or a pathogen borne by a host capable of infecting higher mammals, including humans. In some embodiments, a subject in need can be a healthy subject exposed to or at risk of being exposed to a pathogen. In some embodiments, subjects in need include those already suffering from the disease or disorder caused by a pathogen infection or those diagnosed with a pathogen infection.

Accordingly, the vaccine composition can be administered in advance of any symptom, for example, in advance of a pathogen infection. The vaccine composition can also be administered at or after the onset of a symptom of disease or infection, for example, after development of a symptom of infection or after diagnosis of the infection.

The phrase “therapeutically effective amount” as used herein means that the amount of the multivalent carrier complex disclosed herein which is effective for producing some desired therapeutic effect and/or generating a desired response, such as reduction or elimination of a sign or symptom of a condition or disease, at a reasonable benefit/risk ratio. The therapeutically effective amount also varies depending on the structure and immunogens of the multivalent carrier complex, the route of administration utilized, and the specific diseases or disorders to be treated as will be understood to a person skilled in the art. For example, if a given clinical treatment is considered effective when there is at least a 20% reduction in a measurable parameter associated with a disease or disorder, a therapeutically effective amount of the immunogen—for the treatment of that disease or disorder is the amount necessary to achieve at least a 20% reduction in that measurable parameter.

In some embodiments, a therapeutically effective amount is necessary to inhibit pathogen replication or to measurably alleviate outward symptoms of the pathogen infection or inhibiting further development of the disease, condition, or disorder. In some embodiments, a therapeutically effective amount is an amount that prevents one or more signs or symptoms that can be caused by a pathogen infection. In some embodiments, a therapeutically effective amount can be an amount that prevents one or more signs or symptoms of a particular disease or condition from developing, such as one or more signs or symptoms associated with pathogen infections.

A therapeutically effective amount of the vaccine composition herein described can be estimated from data obtained from cell culture assays and further determined from data obtained in animal studies, followed up by human clinical trials. For example, toxicity and therapeutic efficacy of the vaccine compositions described herein can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices are preferred.

In some embodiments, the determination of a therapeutically effective amount of the vaccine composition can be measured by measuring the titer of antibodies produced against a pathogen. Methods of determining antibody titers and methods of performing virus neutralization arrays are understood by those skilled in the art.

In some embodiments, a method of stimulating an immune response in a subject in need is disclosed herein, the method comprising administering to the subject a pharmaceutically effective amount of the vaccine composition, thereby stimulating an immune response in the subject in need. In some embodiments, administering the vaccine composition induces neutralizing responses against a pathogen.

In some embodiments, administering the vaccine composition induces neutralizing responses against immunogens different from the first immunogen and the second immunogen. In some embodiments, administering the vaccine composition induces neutralizing responses against additional immunogens different from the immunogens from which the plurality of immunogen molecules is derived to produce the vaccine composition. In some embodiments, administering the vaccine composition induces neutralizing responses against the immunogen from which the plurality of immunogen molecules is derived to produce the vaccine composition.

In some embodiments, a method for treating or preventing a pathogen infection in a subject in need thereof is disclosed, the method comprising administering to the subject a pharmaceutically effective amount of the vaccine composition herein described, thereby treating or preventing the pathogen infection in the subject. In another embodiment, a method for treating an infection in a subject in need thereof is disclosed, the method comprising administering to the subject a pharmaceutically effective amount of the vaccine composition herein described, thereby improving the survival rate in the subject. In some embodiments, administering the vaccine composition results in treating or preventing infection caused by a pathogen different from the first pathogen and the second pathogen. In some embodiments, administering the vaccine composition results in treating or preventing infection caused by additional pathogens different from the pathogen from which the plurality of immunogen molecules is derived to produce the vaccine composition. In some embodiments, administering the vaccine composition results in treating or preventing infection caused by the pathogen from which the plurality of immunogen molecules is derived to produce the vaccine composition.

In some embodiments, a method for treating a pathogen infection in a subject in need thereof is disclosed, the method comprising administering to the subject a pharmaceutically effective amount of the vaccine composition herein described, thereby reducing the infectivity in the subject.

In some embodiments, a method of treating or preventing a disease or disorder caused by a pathogen in a subject in need thereof is disclosed, the method comprising administering to the subject a pharmaceutically effective amount of the vaccine composition herein described, thereby treating or preventing the disease or disorder caused by the pathogen in the subject. In some embodiments, administering the vaccine composition results in treating or preventing the disease or disorder caused by a pathogen different from the first pathogen and the second pathogen. In some embodiments, administering the vaccine composition results in treating or preventing the disease or disorder caused by additional pathogens different from the pathogens from which the plurality of immunogen molecules is derived to produce the vaccine composition. In some embodiments, administering the vaccine composition results in treating or preventing the disease or disorder caused by the pathogens from which the plurality of immunogen molecules is derived to produce the vaccine composition.

In some embodiments, the vaccine composition can be used for treating and preventing a broad spectrum of pathogen infections or a disease and disorder caused by such infections by inducing broadly protective anti-pathogen responses. For example, the vaccine composition herein described can elicit broadly neutralizing antibodies that neutralize one or more pathogens from a subfamily, genus, subgenus, species, and/or strain that differ from the subfamily, genus, subgenus, species, and/or strain of the pathogens from which the pathogen immunogens are derived to produce the vaccine composition.

In some embodiments, a method of stimulating an immune response in a subject in need is disclosed herein, the method comprising administering to the subject a pharmaceutically effective amount of the vaccine composition, wherein the vaccine composition administration is independently and individually selected from the group consisting of enteral, parenteral, topical, intravaginal, intranasal, intrarectal, intraocular, and intravitreal thereby stimulating an immune response in the subject in need.

In some embodiments, a method for treating or preventing a pathogen infection in a subject in need thereof is disclosed, the method comprising administering to the subject a pharmaceutically effective amount of the vaccine composition herein described, wherein the vaccine composition administration is independently and individually selected from the group consisting of enteral, parenteral, topical, intravaginal, intranasal, intrarectal, intraocular, and intravitreal thereby treating or preventing the pathogen infection in the subject. In another embodiment, a method for treating an infection in a subject in need thereof is disclosed, the method comprising administering to the subject a pharmaceutically effective amount of the vaccine composition herein described, wherein the vaccine composition administration is independently and individually selected from the group consisting of enteral, parenteral, topical, intravaginal, intranasal, intrarectal, intraocular, and intravitreal thereby improving the survival rate in the subject.

In some embodiments, a method for treating a pathogen infection in a subject in need thereof is disclosed, the method comprising administering to the subject a pharmaceutically effective amount of the vaccine composition herein described, wherein the vaccine composition administration is independently and individually selected from the group consisting of enteral, parenteral, topical, intravaginal, intranasal, intrarectal, intraocular, and intravitreal thereby reducing the infectivity in the subject.

The vaccine composition herein disclosed can be administered to a subject using a single dose or a prime/boost protocol. In an embodiment, the methods herein described can comprise administrating to a subject a vaccine composition once. In another embodiment, the vaccine composition described herein can be administered to the subject in need two or more times. For example, the methods herein described can comprise administering to the subject a first vaccine composition, and after a period of time, administering to the subject a second vaccine composition.

In a prime/boost protocol, a first vaccine composition is administered to the subject (prime) and then after a period of time, a second vaccine composition can be administered to the subject (boost). Administration of the second composition (boost composition) can occur immediately, days, weeks, months, or years after administration of the first composition (prime composition). For example, the boost composition can be administered about simultaneously, three days, 1 week, 2 weeks, 3 weeks, 4 weeks, 5 weeks, 6 weeks, 7 weeks, 8 weeks, 10 weeks, 12 weeks, 16 weeks, 20 weeks, 24 weeks, 28 weeks, 6 months, 1 year, 5 years, 10 years, or a number or a range between any two of these values, after the prime composition is administered.

The prime vaccine composition and the boost vaccine composition can be, but need not be, the same composition. In some embodiments, the prime vaccine composition and the boost vaccine composition can contain the same or different immunogens covalently attached to a multivalent carrier. In some embodiments, the prime vaccine composition and the boost vaccine composition can contain the same immunogens covalently attached to a multivalent carrier, but in different pharmaceutically effective amounts. In some embodiments, the prime vaccine composition and the boost vaccine composition can contain different adjuvants (i.e., different sized linear carbohydrates). In some embodiments the prime vaccine composition and the boost vaccine composition, can be administered by the same method (i.e., both intranasal) or administrated by different methods (i.e., parenteral and intranasal, or enteral and intranasal). In some embodiments, the boost vaccine composition can be administered more than once.

In some embodiments, the boost composition may be comprised of linear carbohydrate with optional vehicles for formulation purposes. That is, the boost composition is free of immunogen and multivalent carriers. In some embodiments, the boost composition may be comprised of the linear carbohydrate covalently attached to the carrier complex in the absence of any immunogen.

The multivalent carrier and the vaccine composition thereof can be used to protect a subject against infection by heterologous pathogens (e.g., pathogens of different taxonomic groups). In other words, a vaccine composition made using immunogens of a first pathogen and immunogens of a second pathogen is capable of protecting an individual against infection by not only the first and second pathogen (i.e., the matched strains), but also pathogens from different taxonomic groups (i.e., mismatched strains or pathogen strains different from the first and second pathogens).

In some embodiments, the multivalent carrier complex and the vaccine composition thereof can protect an individual against infection by an antigenically divergent pathogen. Therefore, in some embodiments, a vaccine composition made using immunogens of a first pathogen and a second pathogen is also capable of protecting an individual against infection by emerging variants of the first and second pathogens.

In some embodiments, the vaccine compositions may also be used in order to prepare antibodies, both polyclonal and monoclonal, for, e.g., diagnostic purposes, as well as for immunopurification of the antigen of interest. If polyclonal antibodies are desired, a selected mammal, (e.g., mouse, rabbit, goat, horse, etc.) is immunized with the vaccine compositions of the present invention (prime). The animal optionally receives a boost 2-6 weeks later with one or more administrations of the vaccine composition. Polyclonal antisera is then obtained from the immunized animal and treated according to known procedures (see, e.g., Jürgens et al. 1985).

The multivalent carrier complex and the vaccine composition containing the multivalent carrier complex as described herein can be provided as components of a kit. Kits can include multivalent carrier complexes or vaccines of the present disclosure as well components for making such multivalent carrier complexes and vaccines. As such, kits can include, for example, primers, nucleic acid molecules, expression vectors, nucleic acid constructs encoding protein immunogens and/or particle-forming subunits described herein, cells, buffers, substrates, reagents, administration means (e.g., syringes), and instructions for using any of said components. Kits can also include pre-formed carriers, linear carbohydrate molecules, and immunogens herein described. In some kits, the linear carbohydrate molecules are already covalently attached to the nanoparticle carrier at some predefined loading. That is, the immunogen or immunogens can be covalently attached at a later point in time. It should be appreciated that a kit may comprise more than one container comprising any of the aforementioned, or related, components. For example, certain parts of the kit may require refrigeration, whereas other parts can be stored at room temperature. Thus, as used herein, a kit can comprise components sold in separate containers by one or more entity, with the intention that the components contained therein be used together.

Structural, chemical and stereochemical definitions are broadly taken from IUPAC recommendations, and more specifically from Glossary of Terms used in Physical Organic Chemistry (IUPAC Recommendations 1994) as summarized by Müller, 1994, and Basic Terminology of Stereochemistry (IUPAC Recommendations 1996) as summarized by Moss, 1996.

For convenience, certain terms employed in the specification, examples and claims are collected here. Unless defined otherwise, all technical and Scientific terms used in this disclosure have the same meanings as commonly understood by one of ordinary skill in the art to which this disclosure belongs.

The initial definition provided for a group or term provided in this disclosure applies to that group or term throughout the present disclosure.

The term “tautomer” as used herein refers to compounds produced by the phenomenon where in a proton of one atom of a molecule shifts to another atom. See March, 1992. Tautomerism is defined as isomerism of the general form:

G-X—Y═Z⇄X═Y—Z-G

where the isomers (called tautomers) are readily interconvertible; the atoms connecting the groups X, Y and Z are typically any of C, H, O, or S, and G is a group which becomes an electrofuge or nucleofuge during isomerization. The most common case, when the electrofuge is H⁺, is also known as “prototropy.” Tautomers are defined as isomers that arise from tautomerism, independent of whether the isomers are isolable.

Oligosaccharides are available in a range of molecular weights. Linear oligosaccharides based on repeating monosaccharide or disaccharide subunits, such as hyaluronic acid and the other glycosaminoglycans (Yeung et al., 2002, Roth et al., 2008, Yamada et al., 2011), may be obtained as discreet sized species or a distribution of sizes about an average molecular weight. Other linear oligosaccharides include chitin, and partially deacetylated chitin (Cho et al., 2000, Cheung et al., 2015). The latter refers to chitin that has been processed to remove some or most of the N-acetyl moieties that modify the glucosamine core. The degree of deacetylation refers to the percentage of N-acetyl moieties removed, with up to 95% removal possible to retain the parent chitin identity. Removal of at least 49% of the acetyl groups within the polymer molecule is required for solubility in water. A range of molecular weights of the parent chitin and the partially deacetylated chitins are possible.

The molecular weight of a linear carbohydrate may be obtained from a variety of direct and indirect physical characterization methods. These include, but are not limited to, high performance liquid chromatography (HPLC), gel permeation chromatography (GPC), size-exclusion chromatography (SEC), mass spectroscopy (MS) including matrix assisted laser desorption ionization mass spectroscopy (MALDI-MS), ultracentrifuge sedimentation, viscosity, osmotic pressure, and dynamic light scattering (DLS), all of which may be affected by the number, size, or shape of molecules in a matrix, a suspension, or in a solution (Orviskf et al., 1991, Motohashi et al., 1984, Yeung et al., 1999, Harding et al., 1991).

For distributions of different sized linear carbohydrates, weight-average molecular weight (Mw) and molecular weight distributions may be determined from ultracentrifuge sedimentation, diffusion, and light scattering. Number-average molecular weight (Mn) and molecular weight distributions may be determined from osmotic pressure and intrinsic viscosity determinations. Optical properties are best reflected in the weight-average molecular weight, while strength properties are best reflected in number-average molecular weight. Mn is the number of monomer molecules divided by the total number of molecules times the monomer mole weight. Mw is the area under the weight distribution curve that is divided into two equal parts. Mn≤Mw.

Weight-average molecular weight, is calculated by the equation

M_{w}={Sum[(Wᵢ)(MW)ᵢ]}/{Sum Wᵢ},

where Wᵢ is the weight fraction of each size fraction and (MW)ᵢ is the mean molecular weight of the size fraction. The “weight-average” molecular weight is particularly significant in the analysis of properties such as viscosity, where the weight of the molecules is important. Number-average molecular weight is calculated by the equation

Mₙ={Sum[(Wᵢ)(MW)ᵢ]}/{Sum Xᵢ},
where the value Xᵢ is the number of molecules in each size fraction.

While various embodiments have been disclosed herein, other embodiments will be apparent to those skilled in the art. The embodiments disclosed herein are for purposes of illustration and are not intended to be limiting. Embodiments disclosed herein focus on the immunogen portion presented by a nanoparticle carrier, but it is understood that the peptide tag covalently attached to a linear carbohydrate molecule is also attached to the nanoparticle carrier.

The following examples set forth preferred methods in accordance with the invention. It is understood, however, that these examples are provided by way of illustration, and nothing therein should be taken as a limitation upon the overall scope of the invention.

### EXAMPLES

The following examples set forth preferred methods in accordance with the invention. It is understood, however, that these examples are provided by way of illustration, and nothing therein should be taken as a limitation upon the overall scope of the invention.

The SpyTag sequence may be referred to as st-003 and the SpyCatcher sequence may be referred to as sc-003. 6×His-SpyTag-RBD(beta) (hereafter referred to as st003-RBD(beta)) was produced using the method of Cohen et al., 2021a. Other st003-RBD proteins were also produced using the method of Cohen et al., 2021a. Sc003-mi3 protein nanoparticle was produced and purified from E. coli according to the method of Tan et al. LPS was removed from sc003-mi3 by repeated Triton X-114 cloud point precipitation as described by Tan et al. Acetonitrile may be referred to as ACN. Hyaluronic acid may be referred to as HA or HyA. Triethylamine may be referred to as TEA. Tris-hydroxvpropyhriazolyliiethyliamine may be referred to as THPTA.

Protein expression and purification: Proteins were either purchased from commercial vendors or prepared by standard recombinant over-expression techniques using published methods (Eschenfeldt et al., 2009) in E. coli (Ausubel, 2003), or Pichia Pastoris (Zonneveld et al., 1995, Ahmad et al., 2014,), and purified by standard methods (Qiagen, 2003).

Carbohydrates: N-acetylglucosamine (GlcNAc), N-acetylmannosamine (ManNAc), N-acetylgalactosamine (GalNAc), diacetylchitobiose (DACB), triacetylchitotriose (TACT), and hyaluronic acid polymers of various lengths including those of less than 6K Mw (HyA6), those of less than 25K Mw (HyA25), those of less than 50K Mw (HyA50), and those of less than 110K Mw (HyA110) were obtained from commercial sources. Other linear carbohydrates are available either from commercial sources or by isolation and purification form natural sources. All samples were screened for the absence of LPS.

Example 1: Preparation of hyaluronic acid tetrasaccharide, free acid form. Hyaluronic acid tetrasaccharide (HyA2) was prepared by digesting 2 g of commercial hyaluronic acid sodium salt (12K, Carbosynth) with 200 mg of bovine type I-S testicular hyaluronidase (Sigma) in 50 mL of 100 mM NaPO₄, pH 6.0 buffer under sterile conditions at 37° C. for 96 h. The tetrasaccharide was purified from the digest by serially injecting 1.5 mL of the sample onto a Superdex 30 26×1000 mm size-exclusion column (SEC) equilibrated in 50 mM triethylamine·acetate pH 7.00 buffer at a flow rate of 2 mL/min. Solvent and triethylamine·acetate were removed under reduced pressure at 40° C. followed by repeated washes with toluene until a dry powder was obtained, which was subsequently washed with CH₂Cl₂ (3×10 mL). The residue was dissolved in 5 mL of distilled water and was passed over a gravity column packed with 3 mL of Dowex-8W H+ form to convert the triethylamine salt to the free acid. Hydrophobic contaminants were removed from the sample by addition of ACN to 10% v/v and passing the solution over a 3 mL C₁₈ functionalized silica column (Waters, Redi-Sep). The solution was snap frozen in a 20 mL scintillation vial with liquid N₂, the water removed by lyophilization, and the resulting white powder stored at −20° C.

Example 2—Preparation of SpyTag peptides. To the st003 peptide as described by Rahikainen, 2021 was added a serine, serine, glycine sequence on the N-terminus, and terminated with a propargyl glycine moiety to create the st003-propargyl peptide. To the st003 peptide as described by Rahikainen, 2021 was added a 6×-histidine tag followed by a serine, serine, glycine sequence on the N-terminus, and terminated with a propargyl glycine moiety to create the 6×His-st003-propargyl peptide. To the st003 peptide as described by Rahikainen, 2021 was added a glycine, serine, serine, glycine sequence on the N-terminus and the C-terminal lysines were Boc protected to create the st003-Boc peptide. All peptides were synthesized using standard methods and purified to at least 95% purity by preparative C₁₈ HPLC before being converted to the acetate salt, lyophilized, and stored under N₂ in crimp seal vials at −80° C. pending use.

Example 3—Preparation of HyA2-st003. HyA2 (10 mg) was dissolved to a concentration of 50 mM in ddi H₂O in a 1 mL glass via, cooled to 0° C. with stirring. 9 mEq of triethylamine and 3 mEq of 2-Chloro-1,3-dimethylimidazolinium chloride (DMC), relative to the HyA2, were added to the vial. The reaction mixture was stirred for 15 min after which 1 mg of st003-Boc peptide was added. The reaction stirred for 0.5-24 h at RT. The solvent was then removed under reduced pressure at 50° C. and the residue dissolved into 200 uL of 10% ACN/90% H₂O+0.1% trifluoroacetic acid (TFA), and purified using a C₁₈ spin column (Pierce). The C₁₈ eluate was analyzed by direct infusion ESI-MS. A peak corresponding to the product was observed in the mass spectrum at an m/z of 1058 in negative mode. The Boc protecting groups were removed by incubation in 10% TFA/H₂O for 10 minutes at 60° C. Successful removal of the Boc groups was confirmed by ESI-MS.

Example 4—Preparation of HyA2-triazole-st003. HyA2 azide was prepared using a modified form of Fairbank's et al.'s method. All molar equivalents are relative to the oligosaccharide starting material. In a 2 mL crimp seal vial with a stir bar, 5 mg of HyA2 was dissolved to 50 mM in a 1:4 mixture of D₂O and acetonitrile and cooled to 0° C. with stirring. TEA (10 mEq) followed by 2-azido-1,3-dimethylimidazolinium hexafluorophosphate (ADMP, 3 mEq) was added to the vial. After 30 min, the reaction was removed from the ice bath and acidified by the careful addition of 1 M HCl until the pH of the reaction was at or below pH 2. The reaction was allowed warm to RT then returned to the ice bath and another cycle of TEA (10 mEq) and ADMP (3 mEq) added to the reaction mixture. This process was repeated three more times, and the reaction neutralized pH 7 by addition of a saturated NaHCO₃ solution. The vial was sealed and the reaction with purged with N₂ gas for 10 min with stirring. While maintaining the N₂ flow through the vial, copper sulfate (0.1 mEq) precomplexed in water with THPTA (0.3 mEq) at 60 mM was added via syringe followed by a 10% w/v solution of 2 mg of st003-propargyl peptide dissolved in water. The reaction between the HyA azide and the st003-propargyl peptide was initiated by addition of ascorbic acid (0.15 mEq) as a 10% w/v H₂O solution. The reaction was sealed and stirred at RT overnight then diluted 10:1 into 10% ACN/H₂O+0.1% formic acid and purified from the peptide and oligosaccharide starting material by preparative C₁₈ HPLC (30×150 mm C18 Gemini column, Phenomenex). Fractions containing the product were pooled, concentrated in vacuo, flash frozen in a 20 mL scintillation vial, and lyophilized overnight.

Example 5—Assembly of st003-triazole-sc003-mi3. St003-propargyl peptide (200 ug) was combined with sc003-mi3 (500 ug) in phosphate buffered saline at 1 mg/mL. The mixture was allowed to react for 1 h at RT then purified by SEC on a Superdex 30 Increase 10×300 mm column equilibrated in PBS. The void volume containing the particle was collected.

Example 6—Assembly of HyA2-st003-triazole-sc003-mi3. The residue in the vial resulting from preparation of HyA-2-triazole-st003 was dissolved into 500 uL of distilled water. A 400 uL portion of the solution was combined with 500 ug of sc003-mi3 in phosphate buffered saline at 1 mg/mL and allowed to react for 1 h at RT, then purified by SEC on a Superdex 30 Increase 10×300 mm column equilibrated in PBS. The void volume containing the particle was collected.

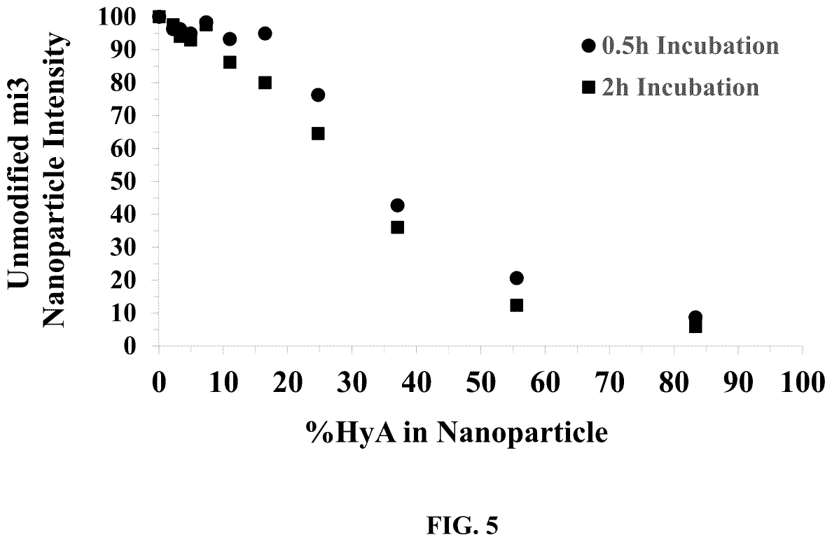

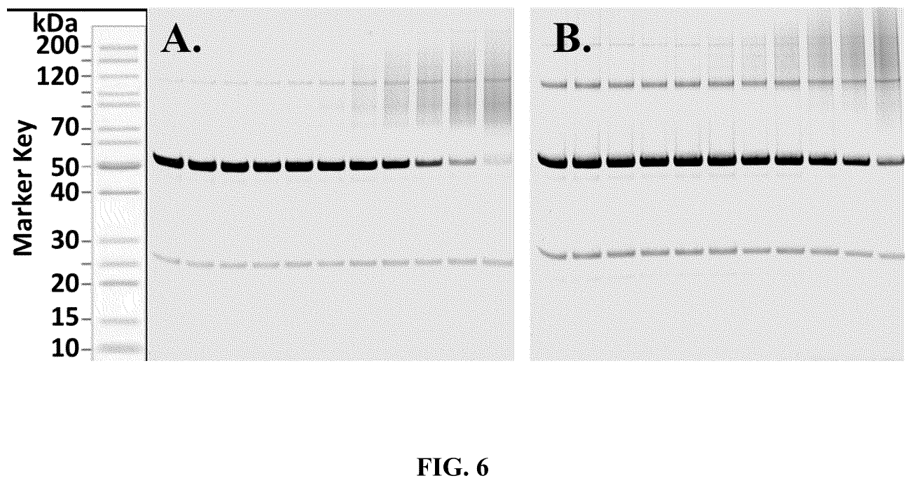

Example 7—DLS analysis of sc003-mi3, st003-triazole-sc003-mi3, and HyA2-st003-triazole-sc003-mi3. A 200 uL portion of Sc003-mi3 at 1 mg/mL was buffer exchanged into PBS by a Sephadex G-20 spin column and the protein concentration of the eluate determined by Bradford assay. A 1 mL sample of sc003-mi3 at 0.1 mg/mL was then prepared by dilution with PBS. Samples of HyA2-st003-triazole-sc003-mi3 and st003-triazole-sc003-mi3 at 0.1 mg/mL in PBS were prepared in an identical fashion. The samples were centrifuged at 17,000 rcf for 5 min then transferred to 3 mL four walled disposable sizing cuvettes (Sarstedt). For each sample, the cuvette was temperature equilibrated in a Malvern instruments Zetasizer Ultra chamber at 25° C. for 5 min after which multiangle dynamic light scattering (MADLS) data were collected. Parameters for index of refraction and viscosity of pure water were used for the analysis and the optimal attenuator was automatically selected by the instrument. After MADLS data collection, the Zeta potential dip electrode was instilled into the cuvette and a zeta potential of the sample determined. The resulting MADLS model of the particle size distribution by intensity is shown in FIG. 5 in an overlay comparison with all other DLS samples. A comparison of the zeta potential measurements is shown in FIG. 6.

Example 8—Intact protein mass spec analysis of sc003-mi3. Sc003-mi3 at 1 mg/mL in PBS was purified by SECon a Superdex 30 increase 10×300 mm column equilibrated in 100 mM NH₄-acetate pH 7.00+30% ACN. Sc003-mi3, eluting from the SEC in the void volume, was diluted 10:1 into 50% CAN/H₂O+0.1% formic acid before direct infusion into an ESI-MS (Velos Pro, Thermo). The intact protein spectrum was collected in positive mode and the most intense charge state peaks were processed by the ESI-prot computer program to determine the molecular weight of the native nanoparticle subunits. The deconvoluted MW of the subunits (36,933 g/mol) was found to be in good agreement with the theoretical MW.

Example 9—Intact protein mass spec analysis of st003-triazole-sc003-mi3. St003-triazole-sc003-mi3 was processed and analyzed as in Example 8. The deconvoluted MW of the subunits (39,228 g/mol) was found to be in good agreement with the theoretical MW.

Example 10—Intact protein mass spec analysis of HyA2-st003-triazole-sc003-mi3. HyA2-st003-triazole-sc003-mi3 was processed and analyzed as in Example 8. The deconvoluted MW of the subunits (40,030.15 g/mol) was found to be in good agreement with the theoretical MW.

Example 11—Preparation of HyA(12K) azide. HyA(12K) azide was prepared in a manner similar to Example 4. All molar equivalents are relative to the HyA starting material. In a 25 mL round bottom flask with a stir bar, 500 mg of hyaluronic acid sodium salt (12K, CarboSynth) was combined with 10 mL of D₂O and 10 mL of DMSO. While cooling the stirred solution to 0° C., TEA (18 mEq) followed by ADMP (3 mEq) were added to the flask. After 1 h, the reaction was removed from the ice bath and acidified by the careful dropwise addition of 1 M HCl until the pH of the reaction was at or below pH 2. The reaction was allowed to proceed at RT for 5 min before being returned to the ice bath. Addition of TEA (18 mEq) and ADMP (3 mEq) followed by the pH/temperature process was performed three more times. After 1 h, the reaction was once more acidified as described above before being neutralized to pH 7 by addition of saturated NaHCO₃ solution. The reaction was then loaded into 6-8 kDa cutoff dialysis tubing and dialyzed against 4 L of distilled water. The distilled water was exchanged at intervals of no less than 12 hours apart three times for a total dialysis solution volume of 4×4 L. At least 12 hours after the final exchange, the sample was recovered from the dialysis bag and shell froze with liquid nitrogen in a lyophilization flask and lyophilized overnight. Total yield relative to the mass of the starting HyA material was 80% and the material was stored at −20° C.

Example 12—Reaction of st003-propargyl peptide with HyA(12K) azide. To a 20 mL septa top scintillation vial with a stir bar was added 9 mL of 20 mM NaPO₄ at pH 7.0 and 1 mL DMSO. The solution was purged with N₂ while stirring for 10 min. Under N₂, 10 mg st003-propargyl peptide and 100 mg of HyA(12K) azide were added to the vial and the solution purged with N₂ while stirring for 10 min. 2 mEq of CuSO₄ (2 mEq) pre-complexed in water with the copper binding ligand THPTA (10 mEq) was then added. The formation of the triazole adduct was initiated by the addition of ascorbic acid (10 mEq) as a 10% solution in H₂O. The reaction was held at 50° C. with stirring and allowed to proceed for 1 h after which the reaction was stopped by the addition of Na-EDTA (4 mEq) as a 0.5 M pH 8.0 solution in H₂O. The ionic strength of the reaction was reduced either by dialysis overnight against 1 mM EDTA or by dilution 5:1 with distilled water. The solution was adjusted to pH to ˜2 with HCl and applied to a 25 mL MacroCap SP cation exchange column equilibrated in 20 mM NaPO₄ at pH 2 at 5 mL/min. The column was washed with 20 mM NaPO₄ at pH 2 until the baseline was stable before being eluted with a step gradient from 20 mM NaPO₄ at pH 2 to 20 mM NaPO₄ at pH 2+0.5 M NaCl. The MacroCap eluate was neutralized to pH 4 by addition of glacial acetic acid before being concentrated to ˜2 mL in an Amicon ultrafiltration device and injected onto a 10×300 mm Superdex 30 Increase column equilibrated in 100 mM ammonium acetate+20% ACN. The column was eluted at 1 mL/min and the void volume peak collected, concentrated, and lyophilized overnight to yield st003-triazole-HyA(12K).

Example 13—Reaction of 6×His-st003-propargyl peptide with HyA(12K) azide. To a 20 mL septa top scintillation vial with a stir bar was added 9 mL of 20 mM NaPO₄ at pH 7.0 and 1 mL DMSO. The solution was purged with N₂ while stirring for 10 min. Under N₂, 10 mg of 6×His-st003-propargyl peptide and 100 mg of HyA(12K) azide were added to the vial and the solution purged with N₂ while stirring for 10 min. 2 mEq of CuSO₄ (2 mEq) pre-complexed in water with the copper binding ligand THPTA (10 mEq) was then added. The formation of the triazole adduct was initiated by the addition of ascorbic acid (10 mEq) as a 10% solution in H₂O. The reaction was held at 50° C. with stirring and the reaction was allowed to proceed for 1 h after which the reaction was stopped by the addition of Na-EDTA (4 mEq) as a 0.5 M pH 8.0 solution in H₂O. The ionic strength of the reaction was reduced either by dialysis overnight against 1 mM EDTA or by dilution 5:1 with distilled water. The solution was adjusted to pH to ˜2 with HCl and applied to a 25 mL MacroCap SP cation exchange column equilibrated in 20 mM NaPO₄ at pH 2 at 5 mL/min. The column was washed with 20 mM NaPO₄ at pH 2 until the baseline was stable before being eluted with a step gradient from 20 mM NaPO₄ at pH 2 to 20 mM NaPO₄ at pH 2+0.5 M NaCl. The MacroCap eluate was neutralized to pH 4 by addition of glacial acetic acid before being concentrated to 2 mL in an Amicon ultrafiltration device and injected onto a 10×300 mm Superdex 30 Increase column equilibrated in 100 mM ammonium acetate+20% ACN. The column was eluted at 1 mL/min and the void volume peak collected, concentrated, and lyophilized overnight to yield 6×His-st003-triazole-HyA(12K).

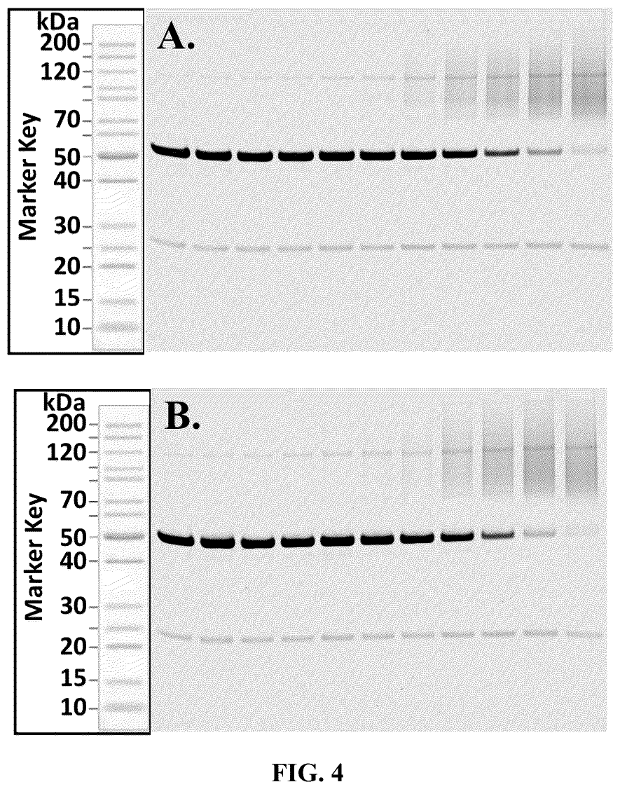

Example 14—Titration of combined 6×His-st003-triazole-HyA(12K) and st003-triazole-HyA(12K) stock solutions with sc003-mi3 with SDS-PAGE evaluation of degree of functionalization. The 6×His-st003-triazole-HyA(12K) and st003-triazole-HyA(12K) stock solutions were pooled. Into a separate microfuge tube 80 uL of the tHis combined solution was dispensed and 60 uL from the tHis tube then diluted by 1.5× via serial dilution with 30 uL portions of PBS eight times. Trial assembly reactions were then set up by dispensing 20 uL of the stock solution of st003-triazole-HyA(12K) and each dilution into separate microfuge tubes followed by dispensing 2 uL of a 2 mg/mL solution of sc003-mi3 in PBS to each tube. These trial assembly reactions were then incubated at 37° C. in a thermocycler and 15 uL samples were withdrawn at 30 minutes and 2 h for SDS-PAGE evaluation of the degree of conversion of sc003-mi3 to st003-triazole-HyA(12K)-sc003-mi3. SDS-PAGE samples were prepared by combining each timepoint sample with 5 uL of 4×LDS sample buffer (Invitrogen) before heating the samples at 98° C. for 5 minutes. SDS-PAGE samples were separated on a 8% bis-tris acrylamide gel (Invitrogen, Bolt) using MES-SDS running buffer. SDS-PAGE gels were run at 200V for 20 min before being stained for 1 h with instant-blue Coomassie based gel stain. After staining, gels were stained overnight in distilled water. Consumption of sc003-mi3 by reaction with st003-triazole-HyA(12K)/6×His-st003-triazole-HyA(12K) at each dilution was then quantified by fluorescence signal analysis of the Coomassie stain at 700 nm using a fluorescence gel imager (Odyssey® M, Li-Cor). Signal integration and background subtraction was performed with Empiria studio 3.0 software (Li-Cor). Raw and normalized fluorescence intensities of the sc003-mi3 band relative to the effective volume of undiluted st003-triazole-HyA(12K)/6×His-st003-triazole-HyA(12K) stock solution used in the conjugation per ug of sc003-mi3 in the assembly reaction were determined. The processed image of the fluorescence data was used to generate a standard curve showing the integration and background areas are shown in FIGS. 4 and 5. Specific loadings of st003-triazole-HyA(12K)/6×His-st003-triazole-HyA(12K) onto the mi3 scaffold could then be accomplished by choosing the appropriate dilution that corresponds to the amount of hyaluronic acid that is desired on the nanoparticle scaffold.

Example 15—Titration of 6×His-st003-triazole-HyA(12K) with sc003-mi3 with SDS-PAGE evaluation of degree of functionalization. The st003-triazole-HyA(12K) from Example 13 was processed in a manner similar to that in Example 14. SDS-PAGE gel analysis results were similar to those shown in FIGS. 4 and 5.

Example 16—Titration of st003-triazole-HyA(12K) with sc003-mi3 with SDS-PAGE evaluation of degree of functionalization. The st003-triazole-HyA(12K) from Example 12 was processed in a manner similar to that in Example 14. SDS-PAGE gel analysis results were similar to those shown in FIGS. 4 and 5.

Example 17—Preparation of 5%, 10%, and 20% HyA(12K) loaded SC003-mi3-RBD(Beta) nanoparticles. Sc0003-mi3 nanoparticle samples with an average of 5%, 10%, and 20% coverage of st003-triazole-HyA(12K)/6×His-st003-triazole-HyA(12K) peptides (hereafter referred to as st003-HyA peptides) were prepared by first dispensing a volume of sc003-mi3 in PBS at 334.9 ug/mL containing 450 ug of protein into each of three microfuge tubes followed by appropriate volumes of the st003-HyA peptide stock solution analyzed in Example 14 to yield 5%, 10%, and 20% coverage of a mi3 nanoparticle with the peptide tag modified with hyaluronic acid (sc003-HyA(12K)). The volume of the stock solution employed to achieve the desired level of functionalization was calculated based on the fluorescence signal analysis as performed in Example 14 which indicated that a trial assembly reaction in which 1.733 uL of the stock solution was incubated with 1 ug of sc003-mi3 for 2 h resulted in, for example, 20% conversion of sc003-mi3 nanoparticles to sc003-HyA(12K)-st003-mi3 nanoparticles with the isopeptide bond forming between SpyTag binding partner in sc003-HyA(12K) and the SpyCatcher binding partner in st003-mi3 (20% sc003-HyA(12K)-st003-mi3 nanoparticle). A similar process was used to create 5% and 10% s sc003-HyA(12K)-st003-mi3 nanoparticle stocks. The assembly was allowed to proceed at 37° C. for 1 h before the addition of 1.5 mEq of 6×His-st003-RBD(beta) protein to each assembly reaction relative to sc003-mi3 subunits present. A control assembly reaction was also prepared containing 600 ug of sc003-mi3 incubated with a 1.5 mEq of 6×His-st003-RBD(beta) in an identical manner but without the addition of st003-HyA peptide solution. Triton X-114 was added to 0.5% v/v and assembly reactions were allowed to proceed overnight at 4° C. The next morning, LPS was removed from the preparation by adding Triton X-114 1% v/v to each assembly reaction. Samples were then incubated at 4° C. for 5 min followed by incubation at 37° C. for 5 min. The samples were then centrifuged at 17,000 rcf for 10 min and the upper phase retained by pipetting it into a clean microfuge tube. The process of Triton X-114 addition, incubation at 4° C. followed by incubation at 37° C., centrifugation, and retention of the upper phase, was repeated two more times. The individual nanoparticle assembly reactions containing the RBD(beta) immunogen and 0%, 5%, 10%, and 20% of the covalently attached HyA modified peptide were then each injected onto a Superose 6 Increase 10×30 mm column equilibrated in TBS+0.75% CHAPS at 1.0 mL/min, and the void volume peaks containing the assembled nanoparticles were collected. Samples were 0.2 um sterile filtered, and the protein mass yield evaluated by Bradford assay. Complete coverage of all sc003-mi3 subunits with either st003-RBD(beta) subunits or st003-HyA peptides as well as removal of free st003-6×His-RBD(beta) from the assembly reactions was confirmed by SDS-PAGE. Endotoxin levels in the final samples were evaluated with an LAL based LPS assay kit (Goldbio, ToxInsensor) and found to be less than 1.5 U/mL for all samples at 100 ug/mL. Representative yields of assembled particles relative to the mass of particles employed in the assembly reactions are shown in Table 1.

Example 18—Sc003-mi3 nanoparticles with 0%, 5%, 10%, and 20% of the covalently attached HyA(12K) modified peptide were reacted with the st003-RBD subunits as described in Example 17 and by Cohen et al., 2021a and purified using published methods.

Example 19—Sc003-mi3 nanoparticles with 0%, 5%, 10%, and 20% of the covalently attached HyA(25K) modified peptide are reacted with the st003-RBD subunits as described in Example 17 and by Cohen et al., 2021a and purified using published methods.

Example 20—Sc003-mi3 nanoparticles with 0%, 5%, 10%, and 20% of the covalently attached HyA(33K) modified peptide are reacted with the st003-RBD subunits as described in Example 17 and by Cohen et al., 2021a and purified using published methods.

Example 21—Sc003-mi3 nanoparticles with 0%, 5%, 10%, and 20% of the covalently attached HyA(50K) modified peptide are reacted with the st003-RBD subunits as described in Example 17 and by Cohen et al., 2021a and purified using published methods.

TABLE 1

| | Mass | | | |
|---|---|---|---|---|
| | sc003-mi3 | | | |
| | starting | Mass recovery | | Yield |
| | material | post assembly | | by mass |
| | (ug) | (ug) | | (%) |
| 0% st003-HyA(12K)-sc003- | 600 ug | 32 | ug | 5.3% |
| mi3-st003-RBD(Beta) | | | | |
| 5% HyA(12K)-sc003- | 450 ug | 72 | ug | 15.8% |
| mi3-st003-RBD(Beta) | | | | |
| 10% HyA(12K)-sc003- | 450 ug | 179 | ug | 39% |
| mi3-st003-RBD(Beta) | | | | |

Example 22—Any one unique st003-RBD subunit as described by Cohen et al. 2021a and sc003-mi3 nanoparticles modified with 0%, 5%, 10%, and 20% of the covalently attached HyA(12K) modified peptide are reacted as described in Example 17 and by Cohen et al., 2021a and purified using published methods.

Example 23—St003-OspC subunits were produced as follows: For each of eight OspC variants included in the mosaic-8-OspC-mi3 nanoparticle, a synthetic double stranded DNA fragment encoding the OspC variant gene+a sequence encoding a 6×His tag+a sequence encoding a st003 or st003 derivative sequence at either the N- or C-terminal region of the OspC coding region with vector targeting sequences at the 5′ and 3′ ends, was purchased from a commercial supplier. The DNA fragment was then cloned by either Gibson assembly or megaprimer PCR mutagenesis into a T7 promotor driven bacterial expression vector. St003-OspC variant expression vectors were then independently transformed into BL21(DE3) E. coli and each protein expressed in shake flask cultures. St003-OspC variants were purified from cellular lysates by immobilized metal ion affinity chromatography and eluted with 300 mM imidazole containing column buffer. Each st003-OspC variant was subsequently purified to homogeneity by SEC.

Example 24—HyA(12K)-mosaic-8-OspC-mi3 nanoparticles was prepared by a method derivative of that employed by Tan et al., 2021 and Cohen et al., 2021a. All eight st003-OspC variants from Example 23 were incubated together with a sub-stoichiometric amount of the sc003-mi3 nanoparticles modified with 0%, 5%, 10%, and 20% of the covalently attached HyA(12K) modified peptide. Assembled HyA(12K)-mosaic-8-OspC-mi3 nanoparticle was separated from free st003-OspC variant proteins by SEC and ion exchange chromatography.

Example 25—HyA(25K)-mosaic-8-OspC-mi3 nanoparticles are prepared and purified as described in Example 24 using st003-OspC subunits from Example 23 and sc003-mi3 nanoparticles modified with 0%, 5%, 10%, and 20% of the covalently attached HyA(25K) modified peptide as described in Example 17.

Example 26—HyA(33K)-mosaic-8-OspC-mi3 nanoparticles are prepared and purified as described in Example 24 using st003-OspC subunits from Example 23 and sc003-mi3 nanoparticles modified with 0%, 5%, 10%, and 20% of the covalently attached HyA(33K) modified peptide as described in Example 17.

Example 27—HyA(50K)-mosaic-8-OspC-mi3 nanoparticles were prepared and purified as described in Example 24 using st003-OspC subunits from Example 23 and sc003-mi3 nanoparticles modified with 0%, 5%, 10%, and 20% of the covalently attached HyA(50K) modified peptide as described in Example 17.

Example 28—HyA(12K)-OspC-mi3 homotypic nanoparticles are individually prepared and purified as described in Example 24 using any one unique st003-OspC subunit from Example 23 and sc003-mi3 nanoparticles modified with 0%, 5%, 10%, and 20% of the covalently attached HyA(12K) modified peptide as described in Example 17.

Example 29—HyA(25K)-OspC-mi3 homotypic nanoparticles are individually prepared and purified as described in Example 24 using any one unique st003-OspC subunit from Example 23 and sc003-mi3 nanoparticles modified with 0%, 5%, 10%, and 20% of the covalently attached HyA(15 kDa) modified peptide as described in Example 17.

Example 30—HyA(33K)-OspC-mi3 homotypic nanoparticles are individually prepared and purified as described in Example 24 using any one unique st003-OspC subunit from Example 23 and sc003-mi3 nanoparticles modified with 0%, 5%, 10%, and 20% of the covalently attached HyA(33K) modified peptide as described in Example 17.

Example 31—HyA(50K)-OspC-mi3 homotypic nanoparticles were individually prepared and purified as described in Example 24 using any one unique st003-OspC subunit from Example 23 and sc003-mi3 nanoparticles modified with 0%, 5%, 10%, and 20% of the covalently attached HyA(50K) modified peptide as described in Example 17.

Example 32—Genes for the production of st003-Chlamydia immunogens subunits are taken from the list including, but not limited to, MOMP serovars D, E, F, G, CPAF, PMPG with the st003 domains, are created as described in Example 22. Production of st003 Chlamydia immunogens is accomplished using standard methods. Open reading frames of proteins are purified from the resulting cultures by contemporary protein chromatography methods such as immobilized metal ion affinity chromatography, immunoaffinity chromatography, size exclusion chromatography, and ion exchange chromatography. Non-SpyTag Chlamydia immunogen variants are also produced using similar methods.

Example 33—HyA(12K)-mosaic-8-Chly-mi3 nanoparticles prepared by a method derivative of that employed by Tan et al., 2021 and Cohen et al., 2021a. All eight st003-Chly variants from Example 32 were incubated together with a sub-stoichiometric amount of the sc003-mi3 nanoparticles modified with 0%, 5%, 10%, and 20% of the covalently attached HyA(12K) modified peptide. Assembled HyA(12K)-mosaic-8-Chly-mi3 nanoparticle was separated from free st003-Chly variant proteins by SEC and ion exchange chromatography.

Example 34—HyA(25K)-mosaic-8-Chly-mi3 nanoparticles are prepared and purified as described in Example 33 using st003-Chly subunits from Example 32 and sc003-mi3 nanoparticles modified with 0%, 5%, 10%, and 20% of the covalently attached HyA(25K) modified peptide as described in Example 17.

Example 35—HyA(33K)-mosaic-8-Chly-mi3 nanoparticles are prepared and purified as described in Example 33 using st003-Chly subunits from Example 32 and sc003-mi3 nanoparticles modified with 0%, 5%, 10%, and 20% of the covalently attached HyA(33K) modified peptide as described in Example 17.

Example 36—HyA(50K)-mosaic-8-Chly-mi3 nanoparticles are prepared and purified as described in Example 33 using st003-OspC subunits from Example 32 and sc003-mi3 nanoparticles modified with 0%, 5%, 10%, and 20% of the covalently attached HyA(50K) modified peptide as described in Example 17.

Example 37—HyA(12K)-Chly-mi3 homotypic nanoparticles are individually prepared and purified as described in Example 33 using any one unique st003-Chly subunit from Example 32 and sc003-mi3 nanoparticles modified with 0%, 5%, 10%, and 20% of the covalently attached HyA(12K) modified peptide as described in Example 17.

Example 38—HyA(25K)-Chly-mi3 homotypic nanoparticles are individually prepared and purified as described in Example 33 using any one unique st003-Chly subunit from Example 32 and sc003-mi3 nanoparticles modified with 0%, 5%, 10%, and 20% of the covalently attached HyA(25K) modified peptide as described in Example 17.

Example 39—HyA(33K)-Chly-mi3 homotypic nanoparticles are individually prepared and purified as described in Example 33 using any one unique st003-Chly subunit from Example 32 and sc003-mi3 nanoparticles modified with 0%, 5%, 10%, and 20% of the covalently attached HyA(33K) modified peptide as described in Example 17.

Example 40—HyA(50K)-Chly-mi3 homotypic nanoparticles are individually prepared and purified as described in Example 33 using any one unique st003-Chly subunit from Example 32 and sc003-mi3 nanoparticles modified with 0%, 5%, 10%, and 20% of the covalently attached HyA(50K) modified peptide as described in Example 17.

Example 41—Genes for the production of soluble recombinant trimeric hemagglutinin (HGA) and soluble recombinant tetrameric neuraminidase (NA) proteins lacking transmembrane domains and the production of soluble recombinant trimeric hemagglutinin (HGA) and soluble recombinant tetrameric neuraminidase (NA) proteins lacking transmembrane domains with the st003 domain are created as described in Example 23. Production of soluble influenza vaccine immunogens derived from HGA and NA, are manufactured by recombinant overexpression in either stably or transiently transfected mammalian cell lines according to the methods described by Ecker et al., 2020. Open reading frames of proteins are purified from the resulting cultures by contemporary protein chromatography methods such as immobilized metal ion affinity chromatography, immunoaffinity chromatography, SEC, and ion exchange chromatography.

Example 42—HyA(12K)-mosaic-8-NAHGA-mi3 nanoparticles are prepared by a method derivative of that employed by Tan et al., 2021 and Cohen et al., 2021a. All eight st003-NAHGA variants from Example 41 were incubated together with a sub-stoichiometric amount of the sc003-mi3 nanoparticles modified with 0%, 5%, 10%, and 20% of the covalently attached HyA(12K) modified peptide.

Assembled HyA(12K)-mosaic-8-NAHGA-mi3 nanoparticle was separated from free st003-NAHGA proteins by SEC and ion exchange chromatography.

Example 43—HyA(25K)-mosaic-8-NAHGA-mi3 nanoparticles are prepared and purified as described in Example 42 using st003-Chly subunits from Example 41 and sc003-mi3 nanoparticles modified with 0%, 5%, 10%, and 20% of the covalently attached HyA(25K) modified peptide as described in Example 17.

Example 44—HyA(33K)-mosaic-8-NAHGA-mi3 nanoparticles are prepared and purified as described in Example 42 using st003-Chly subunits from Example 41 and sc003-mi3 nanoparticles modified with 0%, 5%, 10%, and 20% of the covalently attached HyA(33K) modified peptide as described in Example 17.

Example 45—HyA(50K)-mosaic-8-NAHGA-mi3 nanoparticles are prepared and purified as described in Example 42 using st003-Chly subunits from Example 41 and sc003-mi3 nanoparticles modified with 0%, 5%, 10%, and 20% of the covalently attached HyA(50K) modified peptide as described in Example 17.

Example 46—Genes for the production of soluble Babesia immunogens are taken from the list including, but not limited to, BMSA, BmSA1, BmSP44, BmPROF, BboPROF, BbigPROF, BmAMA-1, BmRON2, Bm2D41, BmSERA1, BmMCFPR1, BmPiβS1, BmBAHCS1, BboPROF, BdAMA1, BdP0, N-terminal and C-terminal fragments of BmRON2, B. microti heat-shock protein-70, Babesia microti methionine aminopeptidase protein 1 with the st003 domains, and are created as described in Example 23. Production of st003 Babesia immunogens is accomplished using standard methods. Open reading frames of proteins are purified from the resulting cultures by contemporary protein chromatography methods such as immobilized metal ion affinity chromatography, immunoaffinity chromatography, SEC, and ion exchange chromatography.

Example 47—HyA(12K)-mosaic-8-Babe-mi3 nanoparticles are prepared by a method derivative of that employed by Tan et al., 2021 and Cohen et al., 2021a. All eight st003-Babe variants from Example 46 were incubated together with a sub-stoichiometric amount of the sc003-mi3 nanoparticles modified with 0%, 5%, 10%, and 20% of the covalently attached HyA(12K) modified peptide. Assembled HyA(12K)-mosaic-8-Babe-mi3 nanoparticle are separated from free st003-Babe proteins by SEC and ion exchange chromatography.

Example 48—HyA(25K)-mosaic-8-Babe-mi3 nanoparticles are prepared and purified as described in Example 47 using st003-Babe subunits from Example 46 and sc003-mi3 nanoparticles modified with 0%, 5%, 10%, and 20% of the covalently attached HyA(25K) modified peptide as described in Example 17.

Example 49—HyA(33K)-mosaic-8-Babe-mi3 nanoparticles are prepared and purified as described in Example 47 using st003-Babe subunits from Example 46 and sc003-mi3 nanoparticles modified with 0%, 5%, 10%, and 20% of the covalently attached HyA(33K) modified peptide as described in Example 17.

Example 50—HyA(50K)-mosaic-8-Babe-mi3 nanoparticles are prepared and purified as described in Example 47 using st003-Babe subunits from Example 46 and sc003-mi3 nanoparticles modified with 0%, 5%, 10%, and 20% of the covalently attached HyA(50K) modified peptide as described in Example 17.

Example 51—Genes for the production of HIV immunogens are taken from the list including, but not limited to, ENV, GP160, Gag, -Pol, -Nef with the st003 domains, and are created as described in Example 23. Production of st003 HIV immunogens is accomplished using standard methods. Open reading frames of proteins are purified from the resulting cultures by contemporary protein chromatography methods such as immobilized metal ion affinity chromatography, immunoaffinity chromatography, SEC, and ion exchange chromatography.

Example 52—HyA(12K)-mosaic-8-HIV-mi3 nanoparticles are prepared by a method derivative of that employed by Tan et al., 2021 and Cohen et al., 2021a. All eight st003-HIV variants from Example 51 are incubated together with a sub-stoichiometric amount of the sc003-mi3 nanoparticles modified with 0%, 5%, 10%, and 20% of the covalently attached HyA(12K) modified peptide. Assembled HyA(12K)-mosaic-8-HIV-mi3 nanoparticle are separated from free st003-HIV proteins by SEC and ion exchange chromatography.

Example 53—HyA(25K)-mosaic-8-HIV-mi3 nanoparticles are prepared and purified as described in Example 52 using st003-HIV subunits from Example 51 and sc003-mi3 nanoparticles modified with 0%, 5%, 10%, and 20% of the covalently attached HyA(25K) modified peptide as described in Example 17.

Example 54—HyA(33K)-mosaic-8-HIV-mi3 nanoparticles are prepared and purified as described in Example 52 using st003-HIV subunits from Example 51 and sc003-mi3 nanoparticles modified with 0%, 5%, 10%, and 20% of the covalently attached HyA(33K) modified peptide as described in Example 17.

Example 55—HyA(50K)-mosaic-8-HIV-mi3 nanoparticles are prepared and purified as described in Example 52 using st003-HIV subunits from Example 51 and sc003-mi3 nanoparticles modified with 0%, 5%, 10%, and 20% of the covalently attached HyA(50K) modified peptide as described in Example 17.

Example 56—Genes for the production of the RSV immunogens are taken from the list including, but not limited to, RSVPreF3 with the st003 domains, and are created as described in Example 23. Production of st003 RSV immunogens is accomplished using standard methods. Open reading frames of proteins are purified from the resulting cultures by contemporary protein chromatography methods such as immobilized metal ion affinity chromatography, immunoaffinity chromatography, SEC, and ion exchange chromatography.

Example 57—HyA(12K)-mosaic-8-RSV-mi3 nanoparticles are prepared by a method derivative of that employed by Tan et al., 2021 and Cohen et al., 2021a. All eight st003-RSV variants from Example 56 are incubated together with a sub-stoichiometric amount of the sc003-mi3 nanoparticles modified with 0%, 5%, 10%, and 20% of the covalently attached HyA(12K) modified peptide. Assembled HyA(12K)-mosaic-8-RSV-mi3 nanoparticle are separated from free st003-RSV proteins by SEC and ion exchange chromatography.

Example 58—HyA(25K)-mosaic-8-RSV-mi3 nanoparticles are prepared and purified as described in Example 57 using st003-HIV subunits from Example 56 and sc003-mi3 nanoparticles modified with 0%, 5%, 10%, and 20% of the covalently attached HyA(25K) modified peptide as described in Example 17.

Example 59—HyA(33K)-mosaic-8-RSV-mi3 nanoparticles are prepared and purified as described in Example 57 using st003-HIV subunits from Example 56 and sc003-mi3 nanoparticles modified with 0%, 5%, 10%, and 20% of the covalently attached HyA(33K) modified peptide as described in Example 17.

Example 60—HyA(50K)-mosaic-8-RSV-mi3 nanoparticles are prepared and purified as described in Example 57 using st003-HIV subunits from Example 56 and sc003-mi3 nanoparticles modified with 0%, 5%, 10%, and 20% of the covalently attached HyA(50K) modified peptide as described in Example 17.

Example 61—Roughly 10⁶ BMDCs were used for each experiment. Femurs and tibia bones from 8-12-week-old BALB/c mice were removed and bone marrow collected into a sterile petri dish containing RPMI media+FBS (10%)+Pen/Strep (complete RPMI). Cells were monodispersed using a pipette, counted, and centrifuged at 300×G for 10 min. Cells were resuspended in complete RPMI with 20 ng/mL of GM-CSF. 2.5×10⁶ cells and added to each well of a 12-well plate and maintained at 37° C. with 5% CO₂. On Day 2, 70% of media was removed and replaced. On Day 3, 2 ug of the HyA-nanoparticle complex was then added to each set of cells. On Day 5, the cells were stained for appropriate cell markers and fixed in 2% paraformaldehyde in PBS. Following 48 hours of exposure to the HyA-nanoparticle, cells were analyzed using flow cytometry to determine the amount of surface expression of maturation markers known to correlate to strong T_{FH} responses including MHC-II, CD80, CD86, OX40 L, and ICOSL. Flow cytometry was accomplished using a BD FACSAria IIIu and FlowJo software for analysis.

Example 62—BALB/c mice (n=5 to 10/cohort) were administered 1, 2.5, 5, or 10 ug equivalent dosages of the HyA(12K)-RBD(beta)-mi3 nanoparticle with and without covalently attached HyA(12K) via 50 ul nasal droplets once per week over a 4-week period. Blood was collected via submandibular bleed post-prime delivery and the serum was analyzed for IgG using standard ELISA techniques. A nasal lavage wash was collected from these same cohorts by administration of 3-4 small drops of sterile saline to a single nostril and extracting the resulting nasopharyngeal wash from the oral cavity using a sterile pipette. Nasal lavage was assayed for sIgA using standard ELISA techniques.

Example 63—Female C3H/HeN mice (n=5 to 10/cohort) were administered a 5 ug protein equivalent of the HyA(12K)-RBD(beta)-mi3 nanoparticle or the RBD(beta)-mi3 nanoparticle IM as a bolus, followed by a 5 ug equivalent IM booster injection at 4 weeks post-prime. Blood was collected via submandibular bleed at 4 and 8 weeks post-prime injection and the serum was analyzed for IgG against the RBD(beta) protein using standard ELISA techniques.

Example 64—Preparation of HyA(50K) azide. HyA(50K) azide was prepared in a manner similar to Example 11. Total yield relative to the mass of the starting HyA material was at least 80% and the material was stored at −20° C.

Example 65—Reaction of st003-propargyl peptide with HyA(50K) azide. This reaction occurred in a manner similar to that for Example 12. After purification and concentration, the material was lyophilized overnight to yield st003-triazole-HyA(50K).

Example 66—Reaction of 6×His-st003-propargyl peptide with HyA(50K) azide. This reaction occurred in a manner similar to that for Example 12. After purification and concentration, the material was lyophilized overnight to yield 6×His-st003-triazole-HyA(50K).

Example 67—Titration st003-triazole-HyA(50K) stock solutions with sc003-mi3 with SDS-PAGE evaluation of degree of functionalization. This sequence occurred as described in Example 14. The SDS-PAGE gel of this process is shown in FIG. 6-B. Specific loadings of st003-triazole-HyA(50K) onto the mi3 scaffold could then be accomplished by choosing the appropriate dilution that corresponds to the amount of hyaluronic acid that is desired on the nanoparticle scaffold.

FIG. 1 shows a schematic of the individual components used to assemble the nanoparticle assembly complex shown as a schematic in FIG. 2. FIG. 2 is a schematic depiction of a carrier with peptide tagged-linker-carbohydrate molecules and peptide tagged-immunogen molecules, where the covalent binding partner, e.g., the binding site for the peptide tag on the carrier or equivalently the site of peptide attachment, is part of each mi3 monomer and forms an isopeptide bond with the peptide tagged-linker-carbohydrate and peptide tagged-immunogen molecules to create the nanoparticle complex. Note that both the linear carbohydrate molecules and the immunogen molecules typically use the same peptide tag.

FIG. 3 is a pictorial representation of the process schematized in FIG. 2 for the mi3 scaffold to create an HyA(50K)-mosaic-8-OspC-mi3 nanoparticle. As shown, the peptide tagged linear carbohydrate, in this instance HyA(50k) is first added to the mi3 nanoparticle to a specified loading, e.g., 5%, 10%, 20%, or some other value, and then the peptide tagged immunogens are, in this instance a mixture of eight different OspC immunogens, are then added to the mi3 nanoparticle mixture to create the HyA(50K)-mosaic-8-OspC-mi3 nanoparticle complex.

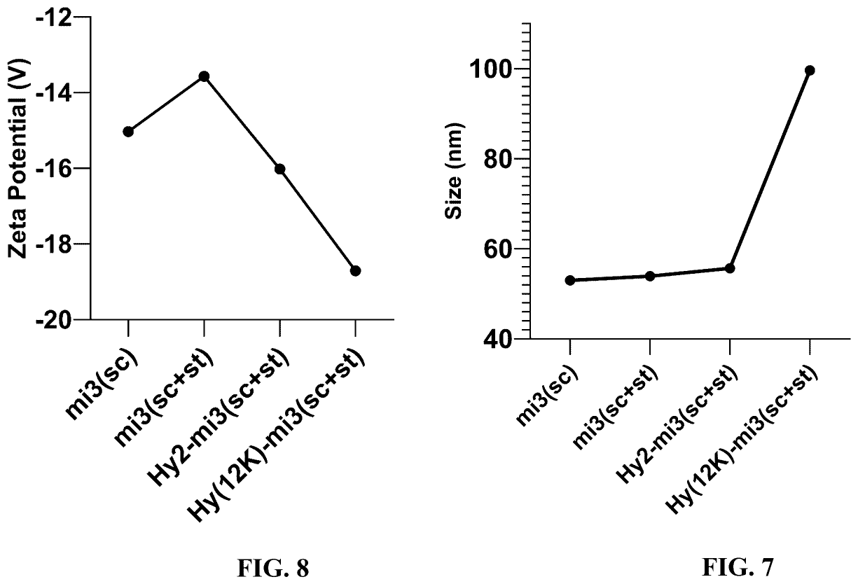

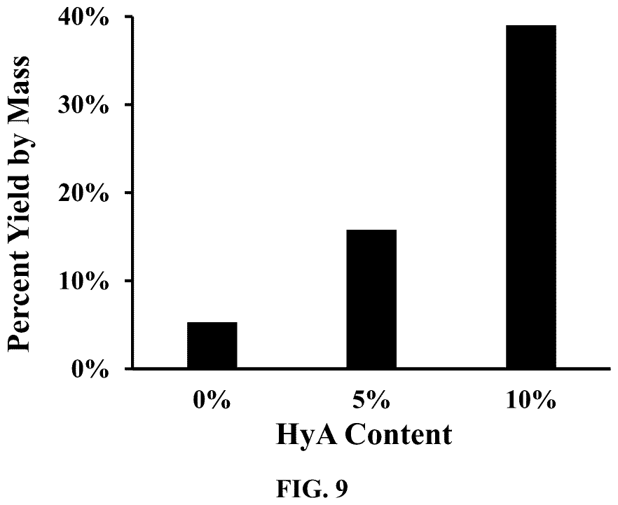

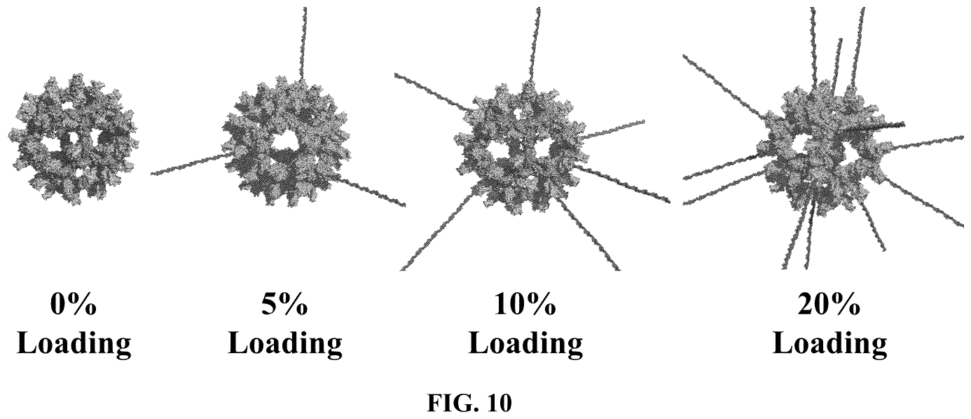

FIG. 4 shows an SDS-PAGE gel titration of st003-HyA(12K) onto a sc003-mi3 nanoparticle with different amounts of HyA(12K) loadings onto the nanoparticle scaffold for (A) after 30 minutes of incubation and (B) after 2 hours of incubation. FIG. 5 shows a LiCor analysis of the SDS-PAGE gels in FIG. 4 demonstrating that the degree of HyA peptide attached to the mi3 nanoparticle can be controlled. FIG. 6 shows the loading of (A) HyA(12K) and (B) HyA(50K) to the mi3 scaffold. Note that the broad band, indicative of HyA attachment, is shifted to higher molecular weight in B, as would be expected because the HyA polymer had, on average, four times the mass as the HyA polymer used in (A). FIGS. 10 and 11 show a schematic rendering of a mi3 scaffold with differing amounts of HyA covalently attached to the nanoparticle through st003-sc003 isopeptide bond and is consistent with the data shown in FIGS. 4, 5, and 6. FIG. 7 shows the change of size of the mi3 nanoparticle as the HyA is added to the mi3 nanoparticle demonstrating that the nanoparticle is larger with the HyA covalently attached. As shown in FIG. 8, as the amount or size of HyA is added to the nanoparticle, the zetapotential of the assembled complex is reduced at pH 7.4 due to the carboxylates on the HyA polymer. Inclusion of the HyA on the nanoparticle can also help improve yields of the assembled product, especially in the absence of detergent or other chaotropes. As shown in FIG. 9, the yield of the final nanoparticle is improved as the amount of covalently attached HyA is added to the mi3 scaffold.

In order for an antigen to be recognized as a threat in vivo and subsequently trigger antibody production, a recognized series of events must occur (Yin et al., 2021) including maturation of dendritic cells, activation of the MHC-II pathway, surface protein expression of CD80/CD86, expression of OX40 L and ICOSL, and activation of T_{FH} and GC responses. The covalent attachment of HyA activates OX40 L and ICOSL via the MHCII pathway. This activation is size dependent as different sized HyA polymers showed different activation. Covalent HyA attachment to the nanoparticle induces maturation and licensing of DCs. HyA50 attachment was superior at expression of these proteins as LPS, which is known to be highly antigenic and served as the positive control. These examples establish that covalently attached HyA is capable of the activation of DCs and production of co-stimulatory surface proteins necessary for optimal T_{FH} and GC responses.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles.

Similarly, a group having 1-5 articles refers to groups having 1, 2, 3, 4, or 5 articles, and so forth. It will be further understood by those within the art that the meaning of the term “about” would be interpreted to refer to an amount that can range +/−10%. Thus, “at least about” would be interpreted to refer to an amount that can range +/−10% (i.e., at least about a molecular weight of 20,000 Da would be at least 18,000 Da). Conversely, “less than about” would be interpreted to refer to an amount that can range +/−10% (i.e., less than about a molecular weight of 20,000 Da would be less than 22,000 Da).

### REFERENCES

Altschul, S. F. et al., J. Mol. Biol. 1990, 215, 403-410.
Baker, T. S. et al., Biophys. J. 1991 60, 1445-1456.
Bashiri, S. et al., Pharmaceutics 2020, 12, 965.
Becker, L. C. et al., Int. J. Toxicol. 2009 July-August; 28(4 Suppl):5-67.
Bottonley, M. J. et al., PCT Pat. Appl. Pub. No. WO 2021/250626.
Brune, K. D. et al., Bioconjugate Chem., 2017, 28(5), 1544-1551.
Brune, K. D. et al., Sci. Rep., 2016, 6, 19234. https://doi.org/10.1038/srep19234
Bruun, T. U. et al., ACS Nano, 2018, 12(9), 8855-8866.
Cheung, R. C. et al., Marine drugs 2015, 13 (8), 5156-86.
Cho, Y.-W. et al., Biomacromolecules 2000, 1 (4), 609-614.
Cohen, A. A. et al., P. J. PLoS ONE 2021, 16(3): e0247963.
Corpet, F. Nuc. Acids Res. 1988, 16(22), 10881-10890.
Current Protocols in Molecular Biology, Ausubel, F. M. et al. Eds.; John Wiley & Sons, 1994-1998.
Das, R et al., Ann. Rev. Biochem. 2008, 77, 363-382.
D'este, M. et al., U.S. Pat. No. 9,034,624, Issued May 19, 2015.
Earnhart, C. G. et al., Human Vaccines, 2007, 3(6), 281-289.
Fairbanks, A. J., Carb. Res., 2021, 499, 108-197.
Fearon D. T. Nature, 1997, 388, 323-324.
Fougeroux, C. F. PCT Pat. Appl. Pub. No. WO 2021/224451.
Gupta, P. N. Devarajan, V. P., Jain, S., Eds.; Springer International Publishing: Cham, Switzerland, 2015; pp. 313-337.
Hagensee, M. E. et al., J. Virol. 1994, 68, 4503-4505.
Harding, S. E. et al., in Advances in carbohydrate analysis. JAI Press: London, England; Greenwich, Conn., 1991, Vol. 1, pp 63-144.
He, L. et al., Science advances, 202 L7(12), 1591.
Hervé, C. et al., NPJ Vaccines, 2019, 4, 39. https://doi.org/10.1038/s41541-019-0132-6
Higgins, D. G. et al., CABIOS 1989, 5(2),:151-153.
Higgins, D. G. et al., Gene, 1988, 73, 237-244.
Howarth, M. PCT Pat. Appl. Pub. No. 2011/098772.
Howarth, M. U.S. Pat. No. 9,547,003,
Howarth, M. U.S. Pat. No. 10,526,379.
Howarth, M. U.S. Pat. No. 10,527,609.
Howarth, M. et al., U.S. Pat. No. 10,889,622.
Howarth, M.; Keeble, A. U.S. Pat. No. 11,059,867.
Howarth, M.; Keeble, A. H. U.S. Pat. Appl. No. 2022/0119459, Published 04/04/2022.
Howarth, M. et al., Appl. No. GB21049999.4.
Hsia, Y.; et al., Nature. 2016, 535(7610), 136-139.
Huang, X.; et al., Bioinfomatics. 1992, 8(2), 155-65,
IUPAC. Compendium of Chemical Terminology, 2nd Ed. Compiled by A. D. McNaught and A. Wilkinson. Blackwell Scientific Publications, Oxford (1997). Online version (2019-) created by S. J. Chalk. ISBN 0-9678550-9-8.
Jurgens, D. et al., J. Chromat. A. 1985, 348, 363-370.
Kang, Y. F. et al., ACS nano. 2021, 15(2), 2738-2752.
Keeble, A. H. et al., Proc. Natl. Acad. Sci. USA. 2019, 116 (52), 26523-26533.
Khairil Anuar, et al., Nat Commun. 2019, 10, 1734.
King, N. P. et al. U.S. Pat. Appl. Pub. No. 2022/0169681.
King, N. P. et al., PCT Pat. Appl. Pub. No. WO 2021/163438.
Kou, S. et al., Nano Res., 2022, 16, 2821-2828.
Kwang, H. S.; et al., Pat. Pub. Appl. No. 2015140149.
Lawson, L. B. et al., Curr. Opin. Immunol. 2011, 23, 414-420.
Li, M. et al., J. Pharm. Investig. 2021, 51, 425-438.
Macpherson, A. J. et al., Mucosal Immunol. 2008, 1, 11.
March, J. Advanced Organic Chemistry: Reactions, Mechanisms and Structures, 4th Ed., John Wiley & Sons, 1992, pp. 69-74.
Mejía-Méndez, J. L. et al., International Journal of Molecular Sciences, 2022, 23(15), p. 8579.
Moss, G. P. Pure Appl. Chem. 1996, 68(12), 2193-2222.
Motohashi, N. et al., Journal of Chromatography A 1984, 299, 508-512.
Müller, P. Pure Appl. Chem. 1994, 66(5), 1077-1184
Molecular Cloning: a Laboratory Manual, 3^{rd} edition; Russell, D. W.; Sambrook, J., Eds.; Cold Spring Harbor Laboratory Press, 2001.
Musheng, Z. et al, PCT Pat. Appl. Pub. No. WO 2022/088953, Published 05/05/2022.
Needleman, S. B. et al., J. Mol. Biol. 1970, 48, 443-453.
Nguyen, B et al., Vaccines, 2021, 6(1), 70.
Noguchi, M. et al., Helvetica Chimica Acta 2012, 95 (10), 1928-1936.
Obata, A.; Ikushima, K. JP Patent No. H015163161A.
O'Hagan, D.; Pavesio, A. U.S. Pat. No. 6,824,793.
Olsen, A. W. et al., The Journal of infectious diseases, 2015, 212(6), 978-989.
Orviskf, E.; et al, Biomedical Chromatography 1991, 5 (6), 251-255.
Pearson, W. R.; Lipman, D. J. Proc. Natl. Acad. Sci. USA. 1988, 85, 2444-2448.
Pearson, W. R. in Computer Analysis of Sequence Data. Methods in Molecular Biology, Griffin, A. M.;
Griffin, H. G. Eds. Humana Press, 1994; vol 24, pp. 307-331.
Petillo, P. A. et al., U.S. Pat. No. 11,021,730.
Petillo, P. A. et al, U.S. Pat. No. 11,643,676.
Rahikainen, R. et al., Angew. Chem. Int. Ed. 2021, 60(1), 321-330
Rosato, A.; Montagner, I. M.; Carpanese, D.; Pieta, A. D. U.S. Pat. Appl. Pub. No. 2021/0393758, Hyaluronic Acid as a Natural Adjuvant for protein and Peptide-Based Vaccines. Published Dec. 23, 2021.
Roth, M. et al., in Carbohydrate Chemistry, Biology and Medical Applications, Garg, H. G.; Cowman, M. K.; Hales, C. A., Eds. Elsevier: Oxford, 2008; pp 209-226.
Simon-Loriere, E. et al., Nat. Rev. Microbiol. 2022, 20, 187-188.
Simon, J. et al., U.S. Pat. No. 6,838,086.
Smith, T. F.; Waterman, M. S. Adv. Appl. Math. 1981, 2, 482-489.
Suzuki, K. et al., Biomater. Sci., 2022, 10, 1920-1928.
Yamada, S. et al., Communicative & Integrative Biology 2011, 4 (2), 150-158.
Yeung, B. K. S. et al., J. Carbohydr. Chem. 2002, 21, 799-865.
Yeung, B. et al, Journal of Chromatography A 1999, 852 (2), 573-581.
Zakeri, B. et a., Proc. Natl. Acad. Sci. USA. 2012; 109 (12), E690-E697.
Zhang, B. et al. Sci. Rep. 2020, 10, 18149.
Zinkernagel, R. M. et al., Immunol Rev. 1997, 156:199-209.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

We claim:

1. A vaccine composition comprising a multivalent carrier covalently attached to one or more linear carbohydrate molecules and a plurality of immunogen molecules, said one or more linear carbohydrate molecules each attached to said multivalent carrier via a reducing end of said linear carbohydrate molecules.

2. The vaccine composition of claim 1, wherein said linear carbohydrate molecules are individually and independently selected from the group comprising chitin, partially deacylated chitin, chitosan, partially acylated chitosan, hyaluronic acid, keratin, keratin sulfate, chondroitin, chondroitin sulfate, dermatan, dermatan sulfate, and heparin, and derivatives thereof.

3. The vaccine composition of claim 2, wherein said linear carbohydrate molecule is hyaluronic acid or a derivative thereof.

4. The vaccine composition of claim 2, wherein said linear carbohydrate molecule has a molecular weight individually and independently selected from the group consisting of about 6,000 Daltons, about 10,000, about 20,000, about 30,000, about 40,000, about 50,000 and about 110,000 Daltons.

5. The vaccine composition of claim 1, wherein said linear carbohydrate molecules and said immunogen molecules are each covalently attached via a respective peptide tag/binding site pair to said multivalent carrier.

6. The vaccine composition of claim 5, wherein there is an optional linker between said peptide tag and said linear carbohydrate.

7. The vaccine composition of claim 1, wherein the number of said linear carbohydrate molecules and the number of said immunogen molecules is in a ratio from about 1:100 to about 100:1.

8. The vaccine composition of claim 1, wherein said plurality of immunogen molecules comprises homologous immunogens.

9. The vaccine composition of claim 1, wherein said plurality of immunogen molecules comprises two, three, four, five, six, seven, eight, nine, ten, eleven, or twelve heterologous immunogen molecules, each of which is different from one another.

10. The vaccine composition of claim 1, wherein said plurality of immunogen molecules is derived from pathogens individually and independently selected from the group consisting of protozoa, fungi, helminths, bacteria, and viruses.

11. The vaccine composition of claim 1, wherein said plurality of immunogens is derived from pathogens independently and individually selected from the group comprising influenza viruses, rhinoviruses, human immunodeficiency viruses (HIV), respiratory syncytial virus (RSV), coronaviruses, dengue viruses, hepatitis viruses, West Nile virus, Middle East respiratory syndrome-related coronavirus (MERS-CoV), norovirus, Marburg viruses, Zika virus, orthopoxviruses, Togaviridae, Ebola virus, Borrelia, Babesia, methicillin-resistant Staphylococcus aureus (MRSA), Legionella, Chlamydia, Plasmodia, Streptococcus pneumoniae, Vibrio cholerae, Listeria, Clostridia, Salmonella, Bordetella, Enterococci, Treponemia, Amoeba, Neisseria, and Giardia.

12. The vaccine composition of claim 1, wherein said multivalent carrier is selected from the group consisting of nanoparticles, nanotubes, nanowires, dendrimers, liposomes, ethosomes and aquasomes, polymersomes and niosomes, foams, hydrogels, cubosomes, quantum dots, exosomes, macrophages, and combinations thereof.

13. The vaccine composition of claim 12, wherein said nanoparticle comprises a virus-like particle (VLP).

14. The vaccine composition of claim 13, wherein said virus-like particle is mutated Ap205 VLP.

15. The vaccine composition of claim 12, wherein said nanoparticle is a self-assembling nanoparticle comprised of a plurality of particle-forming proteins.

16. The vaccine composition of claim 15, wherein said self-assembling nanoparticle comprises a plurality of particle-forming proteins of 2-dehydro-3-deoxy-phosphogluconate (KDPG) aldolase or a variant thereof.

17. The vaccine composition of claim 16, wherein said self-assembling nanoparticle is selected from the group consisting of an i301 nanoparticle or a variant thereof, and a mi3 nanoparticle or a variant thereof.

18. The vaccine composition of claim 15, wherein said plurality of immunogen molecules and said one or more linear carbohydrate molecules are each covalently attached to said particle-forming proteins of said self-assembling nanoparticle.

19. The vaccine composition of claim 15 wherein said plurality of immunogen molecules and said one or more linear carbohydrate molecules are each covalently attached to said particle-forming protein of said plurality of particle-forming proteins through a SpyTag/SpyCatcher binding pair.

20. A method of stimulating an immune response in a subject in need thereof, comprising administering to the subject a pharmaceutically effective amount of the vaccine composition of claim 1, wherein said vaccine composition administration is independently and individually selected from the group consisting of enteral, oral, parenteral, topical, intranasal, intravaginal, intrarectal, intraocular, and intravitreal, thereby stimulating an immune response in the subject.

21. A method for treating or preventing an infection in a subject in need thereof, comprising administering to the subject a pharmaceutically effective amount of the vaccine composition of claim 1, wherein said vaccine composition administration is independently and individually selected from the group consisting of enteral, oral, parenteral, topical, intranasal, intravaginal, intrarectal, intraocular, and intravitreal, thereby treating or preventing the infection in the subject.

22. A method for treating an infection in a subject in need thereof, comprising administering to the subject a pharmaceutically effective amount of the vaccine composition of claim 1, wherein said vaccine composition administration is independently and individually selected from the group consisting of enteral, oral, parenteral, topical, intranasal, intravaginal, intrarectal, intraocular, and intravitreal, thereby improving the survival rate in the subject.

23. A method for treating an infection in a subject in need thereof, comprising administering to the subject a pharmaceutically effective amount of the vaccine composition of claim 1, wherein said vaccine composition administration is independently and individually selected from the group consisting of enteral, oral, parenteral, topical, intranasal, intravaginal, intrarectal, intraocular, and intravitreal, thereby reducing the infectivity in the subject.

24. A method of treating or preventing a disease or disorder caused by an infection in a subject in need thereof, comprising administering to the subject a pharmaceutically effective amount of the vaccine composition of claim 1, wherein said vaccine composition administration is independently and individually selected from the group consisting of enteral, oral, parenteral, topical, intranasal, intravaginal, intrarectal, intraocular, and intravitreal, thereby treating or preventing the disease or disorder caused by the infection in the subject.

25. The method of claim 20, wherein administering said vaccine composition induces neutralizing and cross-reactive neutralizing responses against additional immunogens different from said immunogens in said plurality of immunogens.

26. The method of claim 20, wherein said vaccine composition is administered to the subject one or more times.

27. The method of claim 26, wherein administering said vaccine composition comprises administering to the subject a first vaccine composition and administering to said subject a second vaccine composition.

28. The method of claim 27, wherein said immunogen molecules from said plurality of immunogen molecules in said first vaccine composition and said second vaccine composition are the same.

29. The method of claim 27, wherein said immunogen molecules from said plurality of immunogen molecules in said first vaccine composition and said second vaccine composition are different.

30. The method of claim 27, where said administration of said first vaccine composition and said second vaccine composition are independently and individually selected from the group consisting of enteral, oral, parenteral, topical, intranasal, intravaginal, intrarectal, intraocular, and intravitreal.

31. The method of claim 27, wherein administering to the subject said second vaccine composition occurs about simultaneously, two, three, four, five, six, seven, eight weeks, 10 weeks, 12 weeks, 16 weeks, 20 weeks, 24 weeks, 28 weeks, 6 months, 1 year, 5 years, or 10 years, after administering to said subject said first vaccine composition.

32. A kit, comprising a multivalent carrier covalently attached to one or more linear carbohydrate molecules and a plurality of immunogen molecules.

33. A kit, comprising a multivalent carrier covalently attached to one or more linear carbohydrate molecules.
